(19) United States
(12) Patent Application Publication
(10) Pub. No.: US 2026/0013746 A1
(43) Pub. Date: Jan. 15, 2026

(54) PATIENT-WEARABLE DEVICE FOR DETECTING A SUBPULSE OF A PATIENT AND RELATED SYSTEMS, METHODS AND COMPUTER PROGRAM PRODUCTS

(71) Applicant: Dandelion Medical Devices, Inc., Vero Beach, FL (US)

(72) Inventors: Paul ADAMS, Vero Beach, FL (US); Christopher VETTER, Dublin, OH (US); Alexander PERAIRE-BUENO, Bedford, MA (US); Michael Andrew HOLTMAN, McLean, VA (US)

(21) Appl. No.: 19/335,800

(22) Filed: Sep. 22, 2025

Related U.S. Application Data
(63) Continuation-in-part of application No. 18/617,157, filed on Mar. 26, 2024.
Application No. 18/617,157 is a continuation-in-part of application No. 18/055,052, filed on Nov. 14, 2022.

(51) Int. Cl. A61B 5/024; A61B 5/00; A61B 5/0245; A61B 5/349

(52) U.S. Cl. CPC A61B 5/02438; A61B 5/0006; A61B 5/02405; A61B 5/0245; A61B 5/349

(57) ABSTRACT

A system for determining and indicating whether a patient is or is not exhibiting pulseless electrical activity (PEA) includes a first patient-wearable device attachable to a first body location and operable to generate cardiac signal data, a second patient-wearable device attachable to a second body location and operable to use one or more of an inertial motion sensor or an acoustic sensor thereof to generate pulse wave data, and a processing unit that is configured to receive the cardiac signal data and the pulse wave data, process the cardiac signal data to identify one or more cardiac waveforms thereof, process the pulse wave data to determine whether the pulse wave data includes one or more pulse waveforms that respectively correspond to the identified one or more cardiac waveforms, and selectively generate one of an indication of pseudo-PEA or an indication of PEA in response to at least the determination.

### CROSS-REFERENCE TO RELATED APPLICATIONS

This application is a continuation-in-part of U.S. patent application Ser. No. 18/617,157, filed Mar. 26, 2024, which is a continuation-in-part of U.S. patent application Ser. No. 18/055,052, filed Nov. 14, 2022. The entirety of each of these applications is incorporated by reference herein.

### BACKGROUND

Manual palpation of a pulse, also referred to as a pulse check, is the hallmark of cardiopulmonary resuscitation. Despite its simplicity, few people can accurately determine whether a patient is pulseless within an appropriately short period of time. Studies show that medical practitioners' success rates in rapidly performing a carotid pulse check on a pulseless patient is only in the upper teens (17%), while overall trained medical professionals generally are 55% accurate in manually palpating the presence of a pulse. Further, pulse palpation on individuals on extracorporeal devices has been shown to be only around 78% accurate with a mean time to decision at just over 20 seconds. It has also been reported that only 2% of first responders are able to recognize a truly pulseless patient within 10 seconds of evaluation, while 45% of first responders took 30 seconds to incorrectly determine a patient to be pulseless.

Because the medical mantra “time is tissue” pushes the medical community to minimize time to diagnosis, inaccurate and lengthy pulse detection presents a dilemma for cardiac resuscitation. The hallmark of a common cardiac rhythm during cardiopulmonary resuscitation, pulseless electrical activity (PEA), is in fact reliant on the detection of a pulse while still visualizing a non-perfusing cardiac rhythm on a cardiac monitor. Discordance between a failure to palpate a pulse and the presence of a pulse leads to incorrect treatment management, prolongation of rhythm checks, or even abandonment of resuscitative efforts leading to patient death.

The most common locations for pulse palpation in a critically ill patient are the carotid arteries in the neck and the femoral arteries in the groin. Advanced Trauma Life Support (ATLS) guidelines support that a carotid pulse is palpable at a systolic blood pressure (SBP) of 60-70 mmHg and a femoral pulse at a SBP of 70-80. There are instances, however, where SBP is less than a reliably palpable level and as low as 42 mmHg and 52 mmHg, respectively. Critically, this discrepancy may cause providers to stop resuscitation and pronounce a patient dead with no palpable pulse even though the patient may simply have SBP less than 60 mmHg, and has a blood pressure that is perfusing organs. This scenario exemplifies the clinical “subpulse”—i.e., a spectrum of pulse that is less than reliably manually palpable. Such a patient with cardiac activity and a subpulse needs immediate vasopressor support and additional resuscitation, and not the standard resumption of compressions or cessation of resuscitation, both of which can cause harm. Apart from low SBP, accuracy of pulse and subpulse palpation is further dramatically affected by body habitus, provider experience, environmental stress, and strength of pulse which is directly related to blood pressure but also preexisting vascular disease.

While manual palpation of a pulse remains the guideline standard, recent advancements with use of point of care ultrasound (POCUS) have encouraged some practitioners to use such devices to determine the presence of a pulse. This has been shown to increase pulse detection accuracy to higher levels. POCUS usage, however, presents two key problems. First, it requires an appropriate ultrasound unit to be on hand when a pulse check situation arises, and second, use of the ultrasound requires a dedicated and skilled practitioner, which keeps that practitioner from other resuscitation activities. Use of optical sensors in pulse oximeters is another recent development with the capability to monitor a host patient blood data, including pulse. Multiparameter patient monitor systems employing optical sensors, which typically display the pulse rate, are insufficient alone for pulse checks or in situations with decreased vascular flow. In particular, optical sensors are not adequate for detecting the subpulse. Optical sensors for medical utilization function during optimal conditions, such as minimal subcutaneous tissue between sensor and vessel (radial artery, fingertips, nasal, earlobe), and consistent strength of arterial pulse. Optical sensors are suboptimal/fail with decreased pulse strength and non-perfusion rhythms within the range of subpulse. Patient variability in blood pressure (strength of pulse), body mass, peripheral vascular disease, skin pigmentation and accessible vascular access limit the reliability of optical sensors and, critically, the unreliability or failure of optical sensors to detect subpulse.

Additionally, the determination of a strength and/or presence of a pulse is a common and vitally important examination practice in patients with peripheral vascular disease, which inflicts over 8 million people in the United States and 200 million globally and is the manifestation of systemic atherosclerosis that progressively occludes arteries with atherosclerotic plaque. A common and important practice is palpation of peripheral pulses during each doctor's evaluation. A decreased or absent pulse from the baseline pulse can be a medical emergency and represent near or total vascular occlusion. Typically, a practitioner will initially attempt to palpate a pulse, however the nature of vascular disease significantly decreases the blood flow to the distal artery, leading to decreased pulse strength and difficulty with manual pulse palpation. A provider may inaccurately reason the pulse is absent, however a subpulse may in fact be present. Current standard of care involves using a vascular doppler ultrasound machine to methodically locate a subpulse. This can be time and labor intensive, and have significant provider variability, as small vascular doppler surface area requires precise knowledge of arterial location. Further, the force applied with the doppler can occlude the pulse that leads to inaccurately concluding the absence of a pulse, and the low strength of a subpulse is reliant on the provider hearing the acoustic signal of the doppler, which is further limited by loud and chaotic environments.

Overall, the current standards for pulse detection and subpulse detection in particular are inaccurate, subjective, and burdensome, the results of which can lead to inappropriate medical decisions and patient harm, especially with critically ill patients.

### SUMMARY

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

A patient-wearable device is described herein for detecting a subpulse of a patient and determining a pulse condition based thereon, as well as related systems, methods and computer program products. In an embodiment, the patient-wearable device includes a base layer comprising a printed circuit board (PCB) and electronics connected thereto, and an adhesive layer that is connected to the base layer, the adhesive layer comprising an adhesive suitable for attaching the patient-wearable device to a location on a body of the patient. The electronics may include one or more sensors that generate sensor data, a computer that is connected to the one or more sensors and processes the sensor data generated thereby to determine the pulse condition of the patient, and a user interface (UI) component that is connected to the computer and controlled thereby to generate a user-perceptible indication of the determined pulse condition. In alternate embodiments, the computer and the UI component may be external to the patient-wearable device and the patient-wearable device may communicate the sensor data to the computer via a wired or wireless connection. In further embodiments, multiple patient-wearable devices may be attached to the patient and concurrently transmit raw or processed sensor data to determine the pulse condition.

Further features and advantages of the embodiments, as well as the structure and operation of various embodiments, are described in detail below with reference to the accompanying drawings. It is noted that the claimed subject matter is not limited to the specific embodiments described herein. Such embodiments are presented herein for illustrative purposes only. Additional embodiments will be apparent to persons skilled in the relevant art(s) based on the teachings contained herein.

### BRIEF DESCRIPTION OF THE DRAWINGS

The accompanying drawings, which are incorporated herein and form a part of the specification, illustrate embodiments of the present application and, together with the description, further serve to explain the principles of the embodiments and to enable a person skilled in the pertinent art to make and use the embodiments.

FIG. 1 illustrates a perspective view of a patient-wearable device for detecting a pulse condition of a patient in accordance with an embodiment.

FIG. 2 illustrates a side view of the device shown in FIG. 1.

FIG. 3 illustrates a perspective view of a patient-wearable device for detecting a pulse condition of a patient that includes at least one light emitting diode (LED) indicator in accordance with an embodiment.

FIG. 4 illustrates an exploded view of an electronic assembly of a patient-wearable device for detecting a pulse condition of a patient in accordance with an embodiment.

FIG. 5 depicts a flowchart of a method for detecting a pulse condition of a patient in accordance with an embodiment.

FIG. 6 illustrates a system for providing detection of a pulse condition of a patient in accordance with an embodiment.

FIG. 7 depicts a sheet of patient-wearable devices for detecting a pulse condition of a patient prior to application thereof in accordance with an embodiment.

FIG. 8 illustrates a side view of a patient-wearable device for detecting a pulse condition of a patient in accordance with an embodiment.

FIG. 9 depicts an exemplary implementation of a computing device that may be used to implement embodiments described herein.

FIG. 10 illustrates a side view of a patient-wearable device for detecting a pulse condition of a patient in accordance with an embodiment.

FIG. 11 illustrates a patient-wearable device for detecting a pulse condition of a patient in accordance with an embodiment.

FIG. 12 illustrates a patient-wearable device for detecting a pulse condition of a patient in accordance with an embodiment.

FIG. 13 illustrates a top and side perspective view of a patient-wearable device for detecting a pulse condition of a patient in accordance with an embodiment.

FIG. 14 illustrates a cross-sectional side view of a patient-wearable device for detecting a pulse condition of a patient in accordance with an embodiment.

FIG. 15 illustrates cross-sectional side view of a patient-wearable device for detecting a pulse condition of a patient in accordance with an embodiment.

FIG. 16 illustrates a cross-sectional side view of a patient-wearable device for detecting a pulse condition of a patient in accordance with an embodiment

FIG. 17 illustrates a cross-sectional side view of a patient-wearable device for detecting a pulse condition of a patient in accordance with an embodiment.

FIG. 18 illustrates a cross-sectional side view of a patient-wearable device for detecting a pulse condition of a patient in accordance with an embodiment.

FIG. 19 depicts a flowchart of a method for selectively activating/deactivating sensors of a plurality of patient-wearable devices that are attached or attachable to different locations on a body of a patient, in accordance with an embodiment.

FIGS. 20A, 20B, 20C and 20D illustrate different patient-wearable device configurations that may be suitable for attachment to respective different locations on a body of a patient, in accordance with embodiments.

FIG. 21 depicts an example electrocardiogram (ECG) waveform that may be detected using an ECG sensor of a patient-wearable device, in accordance with an embodiment.

FIG. 22 depicts an example pulse waveform that may be detected using an inertial motion sensor and/or acoustic sensor of a patient-wearable device, in accordance with an embodiment.

FIG. 23 depicts a flowchart of a method for concurrently measuring and monitoring a perfusion index (PI) for each of a plurality of body locations of a patient, in accordance with an embodiment.

FIG. 24 depicts a flowchart of a method for measuring and monitoring a pulse arrival time (PAT) for a body location of a patient, in accordance with an embodiment.

FIG. 25 depicts a flowchart of a method for measuring and monitoring a pulse transit time (PTT) with respect to two different body locations of a patient, in accordance with an embodiment.

FIG. 26 depicts a flowchart of a method for measuring and monitoring a pulse wave velocity (PWV) with respect to two different body locations of a patient, in accordance with an embodiment.

FIG. 27 depicts a flowchart of a method for measuring and monitoring an upstroke velocity at a body location of a patient, in accordance with an embodiment.

FIG. 28 depicts a flowchart of a method for measuring and monitoring a pulsatility index at a body location of a patient, in accordance with an embodiment.

FIG. 29 depicts a flowchart of a method for determining and indicating whether a patient is exhibiting pulseless electrical activity (PEA) or is not exhibiting PEA, in accordance with an embodiment.

FIG. 30 depicts a flowchart of method for detecting a possibility of an arterial occlusion in a patient and presenting an indication thereof, in accordance with an embodiment.

The features and advantages of the embodiments described herein will become more apparent from the detailed description set forth below when taken in conjunction with the drawings, in which like reference characters identify corresponding elements throughout. In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE EMBODIMENTS

### I. Introduction

The following detailed description discloses numerous example embodiments. The scope of the present patent application is not limited to the disclosed embodiments, but also encompasses combinations of the disclosed embodiments, as well as modifications to the disclosed embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future.

References in the specification to “one embodiment,” “an embodiment,” “an example embodiment,” “another embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In the discussion, unless otherwise stated, adjectives such as “substantially” and “about” modifying a condition or relationship characteristic of a feature or features of an embodiment of the disclosure, are understood to mean that the condition or characteristic is defined to within tolerances that are acceptable for operation of the embodiment for an application for which it is intended.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures and drawings described herein can be spatially arranged in any orientation or manner. Additionally, the drawings may not be provided to scale, and orientations or organization of elements of the drawings may vary in embodiments.

The various embodiments set forth herein are described in terms of exemplary block diagrams and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

Numerous exemplary embodiments are described as follows. It is noted that any section/subsection headings provided herein are not intended to be limiting. Embodiments are described throughout this document, and any type of embodiment may be included under any section/subsection. Furthermore, embodiments disclosed in any section may be combined with any other embodiments described in the same section and/or a different section.

### II. Example Embodiments

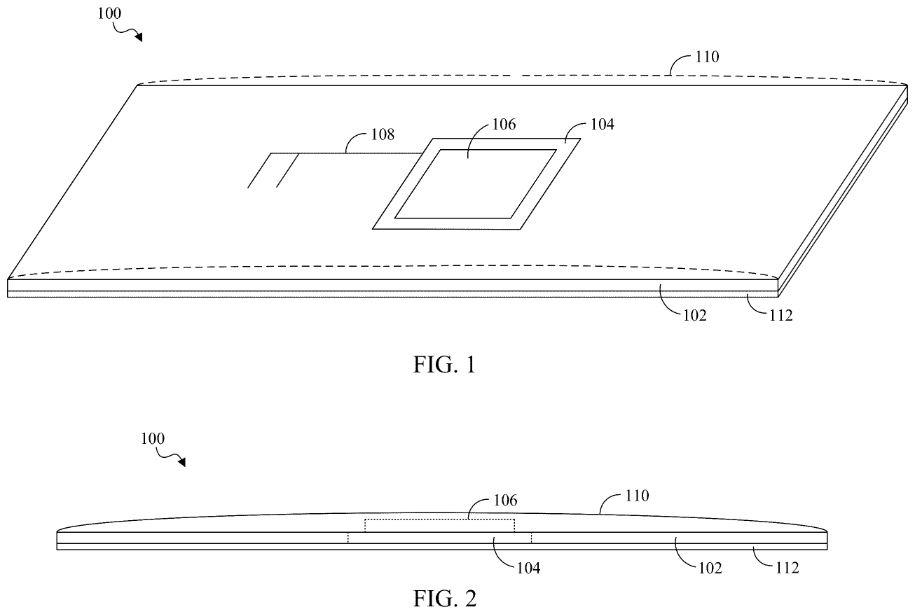

FIG. 1 illustrates a perspective view of a patient-wearable device 100 for detecting a pulse condition of a patient in accordance with an embodiment. As used herein, the term “pulse condition” is intended to at least encompass the presence or absence of a pulse, as well as any characteristics of a detected pulse (e.g., pulse strength) or any characteristics or conditions determinable based on a detected pulse or absence thereof (e.g., heart rate, or presence of an occlusion). FIG. 2 illustrates a side view of device 100.

Device 100 is capable of detecting pulses of various strengths but, importantly, is capable (both through choice of sensor(s) and through post-processing of sensor data, as will be described herein) of detecting subpulses. As used herein, the term “subpulse” refers to a spectrum of pulse that is less than reliably manually palpable. As discussed in the Background Section above, the failure to accurately detect a pulse or determine the absence of a pulse can lead to inappropriate medical decisions and patient harm, especially with critically ill patients.

Device 100 may be of any practicable size as deemed desirable or suitable for a particular application, though it will generally be desirable to have the smallest size useful. As shown in FIGS. 1 and 2, device 100 is rectangularly-shaped. However, embodiments of device 100 can be of any shape. For example, device 100 may be triangular, square, round, oval, or irregularly shaped in nature such as a star configuration or web configuration, which may be optimal for certain procedures. It may be that device 100 is associated with a larger surface area attachment than that shown in FIG. 1 for a desired pulse condition detection. A larger attached system may also contain multiple devices within it that perform the same or separate function as described herein in reference to device 100.

Device 100 comprises a flexible printed circuit board (PCB) 102. Flexible PCB 102 may be a two-layer PCB-however, this is an example only and flexible PCB 102 may comprise only a single layer or more than two layers. As shown in FIGS. 1 and 2, device 100 also includes electronics 106 that are mounted on or otherwise connected to PCB 102. As will be discussed herein, electronics 106 includes one or more sensors and may also include a microcontroller (e.g., for processing sensor data and/or transmitting unprocessed or processed sensor data to an external device or system), and a power source such as a battery. As further shown in FIG. 1, flexible PCB 102 comprises a stiffener 104 in a region thereof to which electronics 106 are connected. Stiffener 104 may provide improved stability and support for electronics 106. Stiffener 104 may be implemented using a material such as FR4, polyamide, aluminum or stainless steel, although these are only examples and are not intended to be limiting. Stiffener 104 may be attached to PCB 102 using thermal bonding, pressure sensitive adhesives, or any other suitable attachment method.

Device 100 further includes an antenna 108 formed on or connected to flexible PCB 102 for enabling unidirectional or bidirectional communication between device 100 (e.g., a microcontroller of device 100) and one or more external devices. Antenna 108 may comprise, for example, a trace antenna that is formed directly on the surface of flexible PCB 102 or a ceramic chip antenna that is mounted on PCB 102.

An overlay 110 to flexible PCB 102 may further be provided and can include any materials that will not interfere with the functioning of electronics 106 and antenna 108, and may protect electronics 106 and antenna 108. In one embodiment, overlay 110 is composed of silicone, although other materials may be used. The thickness of device 100 can vary and may be dictated by a size of a largest contained component, such as a battery, if included. However, it may be deemed desirable to maintain a thinnest thickness achievable for improved flexibility-accordingly, in some embodiments of device 100, such thickness can be in the millimeter(s) range.

In an alternate embodiment of device 100, flexible PCB 102 may be replaced by a flexible base sheet and a smaller semi-rigid PCB (single-layer or multi-layer) may be disposed (e.g., centrally) thereon or therein to support electronics 106. Such semi-rigid PCB may be square shaped, although other shapes may be used. Such semi-rigid PCB may be sufficiently small such that it can align with the contours and surface of a body part to which device 100 is applied. For example, such a semi-rigid PCB may be in the range of 5 to 50 mm square, and in certain embodiments may be in the size range of a 10 to 20 mm square. However, these are merely examples, and the semi-rigid PCB may be several hundred mm square, or other sizes suitable for an intended application. In an embodiment that includes the semi-rigid PCB, the flexible base sheet may support and/or surround the semi-rigid PCB. The material used in the flexible base sheet may be any suitable material for practicing embodiments described herein. The size of flexible PCB 102 or the flexible base sheet may be determined based on factors such as but not limited to increasing adhesion or achieving desired acoustical properties.

As further shown in FIG. 1, device 100 comprises an adhesive layer 112 that enables device 100 to be affixed to the body (e.g., the skin) of a patient. Adhesive layer 112 may comprise, for example and without limitation, a ready-for-use adhesive pad or film. The adhesive used for adhesive layer 112 can be any conventional adhesive appropriate for contact with a patient's skin. In certain embodiments, the adhesive used may also be conductive so as to enable or increase coupling between one or more sensors of device 100 and the patient. Although device 100 is shown as including adhesive layer 112 for affixing device 100 to a body of a patient, other alternative or additional means of maintaining contact between device 100 and a body of the patient can be used. For example, device 100 may be secured to the body using one or more of suction, cuffs, bands, ties, sprays, gravity, clips, etc., so long as interference with the sensors of device 100 is sufficiently low that a desired pulse condition detection function can be achieved.

In embodiments, adhesive layer 112 comprises a replaceable adhesive pad or film that may be attached to device 100 prior to application to a patient. The replaceable adhesive pad or film may be sterile. For example, the adhesive pad or film may be a pre-sterilized disposable component manufactured from relatively inexpensive materials. The pre-sterilized disposable component may be pre-packaged in a suitable packaging material that can be opened at time of use. In accordance with such an embodiment, when a use of device 100 with a particular patient is completed, the pre-sterilized disposable component may be discarded.

Although adhesive layer 112 is shown as being attached to the bottom of PCB 102 in FIG. 1, in alternate embodiments adhesive layer 112 may be disposed across the top of PCB 102 and extend off the sides thereof, or may surround PCB 102 and extend from the sides thereof, so long as adhesive layer 112 is connected (directly or indirectly) to PCB 102 and is enabled to come into contact with a patient's skin such that it can secure PCB 102 thereto.

The aforementioned base sheet and/or adhesive layer 112 of device 100 may be embedded with an additional matter to support device functioning. For instance, the base sheet may be impregnated with electrically conductive material, such as a flexible wire mesh or conductive adhesive, that aids in sensing. An embodiment may be adapted for ECG monitoring. Further, the presence of conductive material in the base sheet or adhesive layer 112 may further aid in communication of device 100 with other devices or external computers. In a still further example, device 100 may utilize the additional material within the base sheet or adhesive layer 112 as a mechanism by which a primary sensor functioning can be amplified. In this scenario, the added material may act to increase the surface area of a primary sensor and its contact points with the body of the patient. Materials in the substrate, or structures on device 100, may also be used to amplify the signal, such as in the case of vibration or sensing done with an accelerometer.

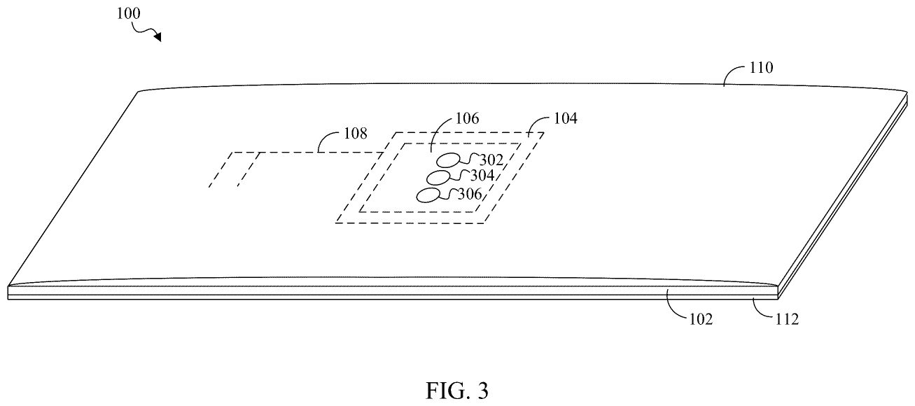

FIG. 3 illustrates a perspective view of an embodiment of device 100 that includes a number of light emitting diode (LED) indicators 302, 304 and 306 in accordance with an embodiment. Although the embodiment shown in FIG. 3 includes three LED indicators, it should be understood that device 100 may include any number of LED indicators as deemed necessary or desirable. Each LED indicator 302, 304 and 306 may be disposed on top of overlay 110 or may be partially or fully disposed in a cavity formed therein, so long as the LED indicator is visible to a practitioner. Furthermore, each LED indicator 302, 304 and 306 may be connected to flexible PCB 102 via a corresponding channel in overlay 110 such that the LED indicator can be powered on or off or otherwise controlled by other component(s) within electronics 106 (e.g., by a microcontroller within electronics 106).

Such LED indicator(s) may be used to for a variety of purposes, such as but not limited to visually indicating a pulse condition of the patient or signifying a status of device 100. A status of device 100 may include, for example, detecting a pulse, streaming (e.g., streaming sensor data to an external computer), powered on, powered off, sleeping (when device 100 supports a low-power sleep mode), functioning, malfunctioning, or the like. Different pulse conditions or statuses may be indicated by using different colors, illumination patterns, degrees of illumination, and/or numbers of LEDs activated.

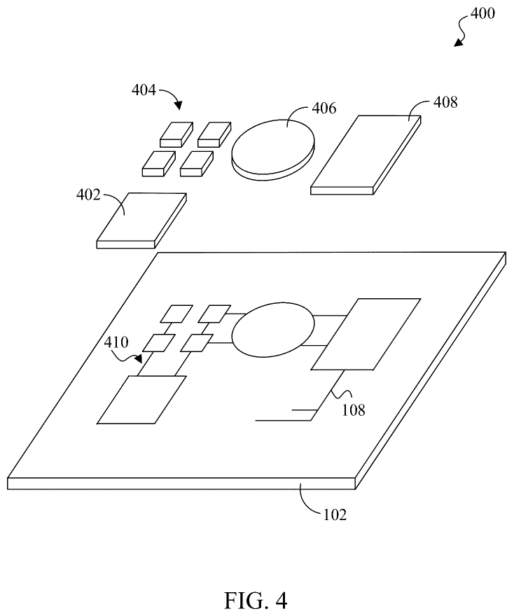

FIG. 4 illustrates an exploded view of an electronic assembly 400 that may be used to implement electronics 106 of device 100 in accordance with one example embodiment. As shown in FIG. 4, electronic assembly 400 includes a number of components that are connected to flexible PCB 102 (e.g., on stiffener 104 of flexible PCB 102) and also electronically connected to each other via a number of PCB traces formed on flexible PCB 102, collectively denoted PCB traces 410. These components include a sensor 402, a number of passive electronic components 404, a battery 406, a microcontroller 408, and antenna 108.

Sensor 402 may comprise any type of sensor suitable for detecting a pulse condition in a patient. In an embodiment, sensor 402 comprises an inertial measurement unit (IMU) that integrates one or more of a multi-axis accelerometer or multi-axis gyroscope and that provides suitable sensitivity to detect a desired pulse in a patient. In another embodiment, sensor 402 comprises an acoustic sensor. However, these are merely examples and other types of sensors may be used for detecting a desired pulse in a patient.

Although only a single sensor 402 is shown in FIG. 4 for the sake of illustration, it is to be understood that device 100 may contain any number of sensors that aid directly in pulse condition detection, or in the detection of other patient qualities or conditions. For example, device 100 may include a primary sensor of a first type (e.g., an IMU) and one or more additional sensors of a second type (e.g., acoustic sensors) that may be used to provide further sensing capabilities and/or to provide checks on the primary sensor. In certain settings, combined data, such as that from both inertial and acoustic sensing, may give enhanced data fidelity because information from each type of sensing is fundamentally different. In yet another embodiment, device 100 may comprise multiple sensors of a same type. For example, device 100 may comprise a plurality of physical accelerometers, each of which generates its own sensor data.

In embodiments, the sensors utilized by device 100 may comprise any one of the following sensor types having a sensitivity (alone or combined with other sensors) suitable for detecting a subpulse: an accelerometer, a gyroscope, a magnetometer, an IMU that comprises one or more of an accelerometer, a gyroscope or a magnetometer, or an acoustic sensor.

As noted above, device 100 may also include sensors for detecting patient qualities or conditions other than a pulse condition. For example, device 100 may include sensors for detection of one or more of blood pressure, blood sugar, blood oxygen (e.g., a pulse oximeter), echocardiogram, body temperature, respiratory rate, blood flow rate, magnetic fields, or the like.

Passive electronic components 404 comprise circuit components that do not require a power source (such as resistors, capacitors, inductors, and the like) and that are used to control the flow of power and electrical signals to the other electronic components that make up electronic assembly 400.

Battery 406 comprises a power source for active electronic components within electronic assembly 400. For example, battery 406 may be used to provide power for sensor 402 and microcontroller 408. In one embodiment, battery 406 comprises a button cell battery, although this is only one example.

Microcontroller 408 comprises an integrated circuit (IC) chip that implements a computer configured to perform various functions relating to detecting a pulse condition in a patient as will be described herein. In an embodiment, microcontroller 408 is wireless-enabled and thus may communicate wirelessly with one or more external devices (e.g., for the purpose of communicating sensor data and/or other information). For example, microcontroller 408 may be capable of communicating with other devices via a Bluetooth® protocol (e.g., as specified by the IEEE 802.15.1 standard), a Wi-Fi® protocol (e.g., as specified by the IEEE 802.11 family of standards), and/or other radio frequency (RF) protocol. Hospital settings may dictate a preferred form of wireless communication for device 100; however, Wi-Fi® is believed to be sufficiently robust in most clinical settings so as to not interfere with other patient devices or equipment.

In various embodiments, device 100 may include a microprocessor, a digital signal processor (DSP), or an application-specific integrated circuit (ASIC) instead of microcontroller 408, or in addition to microcontroller 408, for performing processing tasks.

As shown in FIG. 4, to facilitate the aforementioned wireless communication, electronic assembly 400 includes antenna 108 that is connected to microcontroller 408. In the embodiment shown in FIG. 4, antenna 108 comprises a trace antenna that is formed directly on flexible PCB 102 in a well-known manner. However, this is an example only, and antenna 108 may comprise a ceramic chip antenna or other suitable type of antenna.

In an alternate embodiment, device 100 may be capable of communicating with an external device via a wired connection. For example, in an embodiment, device 100 does not include microcontroller 104 but instead communicates sensor data to an external computer via a wired connection thereto. Such external computer may comprise, for example, and without limitation a microcontroller (e.g., an Intel® 8051 microcontroller), a microcontroller board (e.g., an Arduino® microcontroller board), or a microprocessor-based mini-computer (e.g., a Raspberry Pi® microprocessor-based mini-computer). In an alternate embodiment, the communication of the sensor data to the external computer is carried out via a wireless connection. In still further embodiments, device 100 may include microcontroller 104 and also communicate with an external computer via a wired and/or wireless connection thereto.

Embodiments of device 100 can be applied in all clinical settings, including for use during cardiopulmonary resuscitation (CPR), during cardiac arrest (code), or the moments just prior to or after cardiac arrest (peri-code), on patients with or without forms of vascular disease that impact pulse detection, to detect the presence of a pulse in an extremity for cases of concern for arterial clot, or pulse/heart rate detection in persons, including fetal heart rate/pulse.

In an embodiment, device 100 is suitable for use on a patient for an extended period of time, such as the duration of a stay at a hospital. For example, device 100 may be adapted to have a relatively large internal battery power source, be wired to an external power source, and/or enter an energy-saving rest mode during periods of non-use for activation when a pulse check is required. As another example, adhesive layer 112 may comprise a material that provides for long-term adhesion. Such long-term adhesion could be valuable during hospitalization or for telemedicine to determine dynamic changes of a pulse in real time for immediate provider notification. The clot of an artery (such as a radial artery or femoral artery occlusion) is a true medical emergency and needs to be diagnosed immediately.

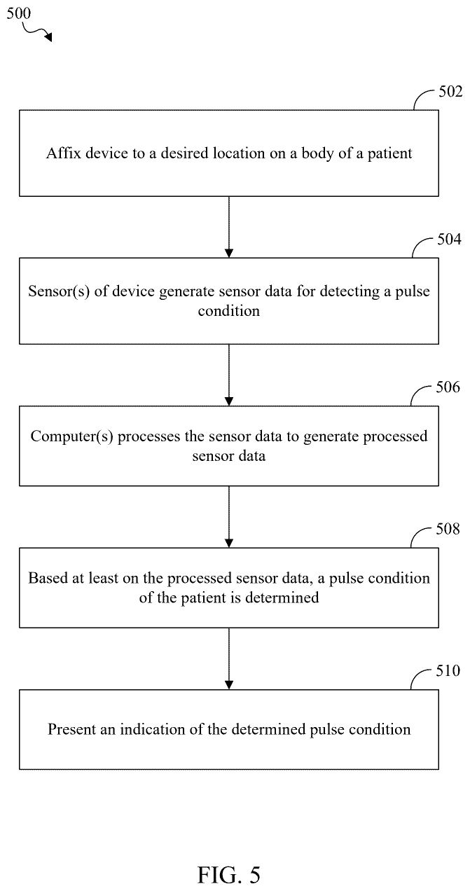

FIG. 5 depicts a flowchart 500 of a method for detecting a pulse condition of a patient in accordance with an exemplary embodiment. As shown in FIG. 5, the method of flowchart 500 begins at step 502 in which device 100 is affixed (e.g., by a practitioner) to a desired location on a body (e.g., on the skin) of a patient. As noted above, an adhesive layer 112 and/or various other means of attachment (e.g., suction, cuffs, bands, ties, sprays, gravity, clips) may be used to secure device 100 to a desired body location.

In embodiments, the size and flexibility of device 100 render it suitable for attachment to most locations on a body of a patient. In embodiments, device 100 may be suitable for attachment to any location on a patient's body, but in accordance with particular embodiments, device 100 can be attached at least over the superficial aspects of the dorsalis pedis (DP) artery (along the dorsal aspect of the foot) and the posterior tibial (PT) artery (posterior to the medial malleolus). By way of further example, device 100 may be placed along the popliteal artery (posterior to the knee in the popliteal fossa) and/or femoral artery (mid to medial aspect of the inguinal ligament (commonly the groin)). Device 100 may also be placed on the chest (possibly near the Point of Maximal Impulse (PMI)), over a carotid artery, over a femoral artery, or over a radial artery. The location of attachment of device 100 can yield different advantages. In some instances, the placement of device 100 may allow the determination of point of occlusion along the lower or upper extremity for example. Device 100 may be suitable for attachment to a body surface over or adjacent to an underlying vascular structure.

At step 504, after device 100 has been affixed to a location in step 502, one or more sensors of device 100 (e.g., sensor 402) generate sensor data for detecting a pulse condition. Such sensor(s) may include, but are not limited to, a multi-axis accelerometer, a multi-axis gyroscope, an IMU (e.g., that incorporates a multi-axis accelerometer and gyroscope), an acoustic sensor, a magnetometer, or any other type of sensor deemed suitable for detecting a pulse condition in a patient.

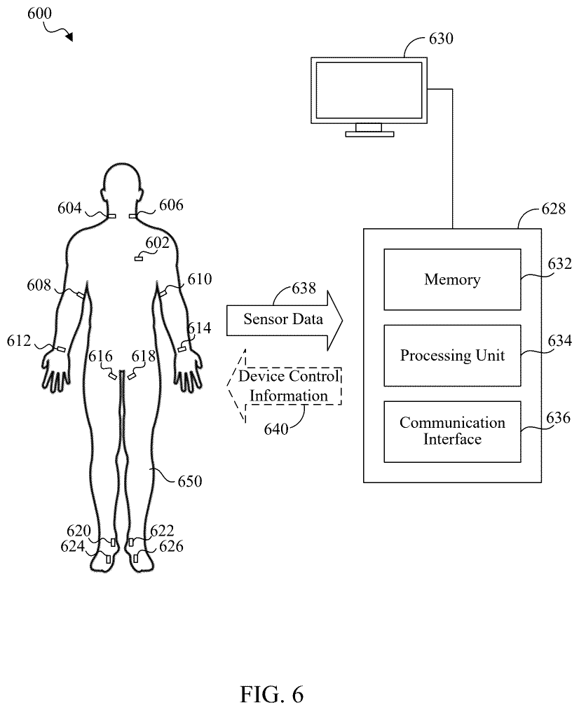

At step 506, the sensor data generated during step 504 is provided to one or more computer(s) and such computer(s) process the sensor data to generate processed sensor data. The computer(s) used to process the sensor data may be located on device 100 (e.g., in the form of microcontroller 408) or may be located externally with respect to device 100, in which case the sensor data generated during step 504 may be transmitted thereto via a wired or wireless connection. Still further, the processing of sensor data may be carried out in a distributed manner by a computer located on device 100 and one or more external computers. Various system implementations that rely on external computers for processing the sensor data will be described below in reference to FIG. 6.

The processing of the sensor data during step 504 may be carried out, for example, to address the issue of background noise, which can originate from a variety of sources in the clinical setting and can reduce overall accuracy of pulse readings. Such background noise may result from surface-level movements of the patient's body, both direct and indirect, as well active electronic monitoring, such as electrocardiograms (ECGs), cardiac monitors, pacemaker/defibrillator pads, and ultrasounds. Background noise can lead to significant rates of false positives where a perceived pulse detection is actually interference with the patient anatomy, such as simply lifting the patient's arm. Background noise can be addressed at least in part through the choice of sensor(s) that generate the sensor data in step 504. However, in embodiments, the issue of background noise is alternatively or additionally addressed through appropriate processing of the sensor data in step 506. For example, the computer(s) that process the sensor data may filter and/or compensate for background noise to reduce such false positive readings. In the case of filtering through sensor selection, complementary sensors that are vulnerable to noise in different domains can be used together to extract the target signal. Processing of the sensor data (e.g., analog or digital signal representations) may also be performed in either or both the time and frequency domains. Strategies may include, but are not limited to, pattern matching with expected heartbeat waveforms, filtering based on key heartbeat waveform attributes (duration, amplitude, etc.), and filtering of key frequencies in the frequency domain.

Filtering of the sensor data in the time domain may include, for example and without limitation, removing noise that is far from an expected heartbeat. For example, in a scenario in which an accelerometer is used, such noise can be removed if there is a large spike in acceleration, which may be more likely due to movement (e.g., a cough) other than a heartbeat. An embodiment can also filter out the effects of movements that are unlike a heartbeat in terms of duration. For example, if there is a spike that lasts much longer than expected, it may be the patient breathing, rather than a heartbeat. The processing can be adjusted to greater and lesser extents depending on what is being looked for in terms of shape and amplitude of a target signal.

Filtering of the sensor data in the frequency domain may include, for example and without limitation, cleaning up a sensor-generated signal with band pass filters, by analyzing dominant frequencies in the signal, or the like. In some embodiments, a combination of filtering in the time domain and filtering in the frequency domain may be used to generate the processed sensor data.

Processing of the sensor data in step 506 may alternatively or additionally comprise comparing and/or combining sensor data generated by multiple different sensors of device 100 or comparing and/or combining sensor data generated by the sensor(s) of device 100 with sensor data provided from other devices that are attached to the patient. An example system that can process sensor data generated by multiple different devices that are simultaneously attached to a patient will be described below in reference to FIG. 6.

The aforementioned processing of the sensor data in step 504 can enhance the ability of device 100 or of a system including device 100 to detect a pulse condition in a patient, including a subpulse.

At step 508, a pulse condition of the patient is determined based at least on the processed sensor data. This step may be performed, for example, by any of the same computer(s) used to process the sensor data in step 506, or by a different computer that receives the processed sensor data therefrom. The determined pulse condition may include, for example and without limitation, a presence or absence of a pulse, a characteristic of a detected pulse (e.g., pulse strength), or a characteristic or condition determinable based on a detected pulse or absence thereof (e.g., heart rate, or presence of an occlusion).

At step 510, an indication of the determined pulse condition is provided (e.g., to a practitioner). For example, a visual and/or auditory indication of the determined pulse condition of the patient can be provided to a practitioner by one or more suitable user interface components of device 100, and/or by one or more suitable user interface components external to device 100.

For example, device 100 may include one or more LED indicators as discussed above in reference to FIG. 3 and such LED indicators may be used to visually indicate a determined pulse condition of a patient. For instance, illumination of a green LED indicator may indicate that a pulse has been detected while illumination of a red LED indicator may indicate that no pulse has been detected. Blinking vs. steady illumination of an LED indicator may also be used to distinguish between detection and non-detection of a pulse. A relatively strong pulse may be indicated by illuminating more LED indicators than would be illuminated for a relatively weak pulse. Likewise, a relatively strong pulse may be indicated by a brighter illumination of an LED indicator while a relatively weak pulse may be indicated by a dimmer illumination of the LED indicator. Furthermore, an LED indicator may be illuminated in a periodic manner that mimics a detected pulse (e.g., with the LED indicator lighting up for each beat of the pulse). However, these are only examples, and persons skilled in the relevant art(s) will appreciate that any of a variety of LED indicator features (e.g., color, illumination pattern, degree of illumination, and/or number of LEDs activated) can be used to indicate a determined pulse condition to a practitioner.

In another example embodiment, device 100 may include a mini- or micro-speaker that is capable of emitting an auditory indicator of a pulse condition. For example, the speaker may emit a sound only if a pulse has been detected. As another example, the speaker may emit a first sound to indicate that a pulse is detected and emit a second sound to indicate that a pulse has not been detected. As yet another example, the speaker may emit sounds that mimic a perceived pulse of the patient upon detection.

In a further example embodiment, a visual and/or auditory indication of the detected pulse condition may be displayed via a display screen and/or speaker associated with an external computer to which device 100 is communicatively coupled via a wireless or wired communication medium.

FIG. 6 depicts a system 600 for providing detection of a pulse condition of a patient in accordance with an embodiment. As shown in FIG. 6, system 600 includes a plurality of patient-wearable devices (i.e., patient-wearable devices 602, 604, 606, 608, 610, 612, 614, 616, 618, 620, 622, 624 and 626) that are concurrently attached to different locations on a body of a patient 650, a computing device 628, and a display device 630.

As further shown in FIG. 6, computing device 628 comprises memory 632, a processing unit 634, and a communication interface 636. Memory 632 may comprise, for example, one or more volatile memory devices (e.g., one or more RAM devices) and/or one or more non-volatile memory devices (e.g., one or more ROM devices, flash memory devices, magnetic storage devices, optical disks, or the like). Processing unit 634 may comprise one or more microprocessors, microcontrollers, DSPs, and/or ASICs. Processing unit 634 may be configured to execute software instructions stored in memory 632 to perform any of the operations attributed to computing device 628 as described herein (e.g., processing of sensor data to generate processed sensor data, the detection of a pulse condition based on sensor data, and/or the generation of a visual or auditory indication of the detected pulse condition). Communication interface 636 may comprise a wired communication interface (e.g., a USB interface) and/or a wireless communication interface (e.g., a Bluetooth®, WiFi® or other RF interface) suitable for receiving sensor data 638 from one, some or all of the wearable devices shown in FIG. 6, and for optionally transmitting device control information 640 to one, some or all of the wearable devices shown in FIG. 6. Display device 630 may comprise a part of computing device 628 (e.g., integrated into a same housing as computing device 628) or may be separate from computing device 628 but connected thereto via a suitable wired or wireless connection.

Each of the patient-wearable devices in FIG. 6 may be implemented in a like manner to device 100 as discussed above. Furthermore, each such device may be capable of transmitting unprocessed and/or processed sensor data 638 to computing device 628 via a wired or wireless connection. Computing device 628 may receive such sensor data 638, e.g., via communication interface 636, and utilize such sensor data 638 to detect a pulse condition in patient 650 (e.g., a pulse condition for each one of the patient-wearable devices). Computing device 628 may then provide an indication of each one of the detected pulse conditions to a practitioner. For example, computing device 628 may cause a visual indication of each one of the detected pulse conditions to be displayed (e.g., concurrently) by display device 630.

The patient-wearable devices may thus be considered additive in nature and can be placed as desired throughout the anatomy of a patient (e.g., patient 650) to perform a specific sensing task. The ability to concurrently detect pulse conditions at different body locations may be particularly beneficial in situations of cardiac arrest and other critical conditions for which rapid pulse detection (or lack thereof) is pivotal. For example, different patient-wearable devices may be concurrently attached to the chest (possibly near the Point of Maximal Impulse (PMI)), over a carotid artery, over a femoral artery, and/or over a radial artery. Several points of contact may increase sensitivity and specificity and allow for additional clinical decisions based on data points and calculations. For example, an aortic dissection may be indicated if pulse strength readings from patient-wearable devices placed on the left side of body are different than pulse strength readings from patient-wearable devices placed on the right side of the body. Multiple points of body contact may increase accuracy and also allow real time data to be collected for oxygenation levels, body temperature, respiratory rate, and change in blood pressure.

In an embodiment, each of the patient-wearable devices shown in FIG. 6 transmits unprocessed sensor data to computing device 628 via communication interface 636, and processing unit 634 of computing device 628 processes and interprets the sensor data to detect a pulse condition. Processing unit 634 then generates a perceptible indication of the pulse condition for presentation to a practitioner, such as a visual indicator for presentation via display device 630 or an auditory indicator for emitting via a speaker integrated with or connected to computing device 628 (not shown in FIG. 6).

In a further embodiment, as each patient-wearable device is activated and placed for use on patient 650, any additional sensor data generated thereby is sent to computing device 628, which combines it with other sensor data and interprets the combined sensor data to generate and present a result (e.g., a perceptible indication of a pulse condition of patient 650). The sensor data may be transmitted to computing device 628 by each patient-wearable device may include an identifier of the patient-wearable device from which it originated. Computing device 628 may be configured to dynamically switch from operating with a single patient wearable-device to operating with multiple patient-wearable devices as new streams of sensor data are received.

In one embodiment of the system shown in FIG. 6, the patient-wearable devices all possess the same type of sensor and thus generate the same type of sensor data. However, in an alternate embodiment, the patient-wearable devices possess different types of sensors and thus generate different types of sensor data. For example, some of the patient-wearable devices shown in FIG. 6 may include an IMU but not an acoustic sensor, while other ones of the patient-wearable devices shown in FIG. 6 may include an acoustic sensor but not an IMU. Furthermore, some of the patient-wearable devices shown in FIG. 6 may include sensors for detecting qualities of the patient other than the presence of a pulse (e.g., blood pressure, blood sugar, blood oxygen, echocardiogram, body temperature, respiratory rate, blood flow rate), while other ones of the patient-wearable devices shown in FIG. 6 may not include such sensors. In further accordance with such embodiments, each patient-wearable device may include an identifier of the type of sensor used to generate the sensor data when transmitting the sensor data to computing device 628.

In one embodiment of system 600, each of the patient-wearable devices is capable of one-way communication with computing device 628 and utilizes such one-way communication to send sensor data 638 thereto. In an alternate embodiment, each of the patient-wearable devices is capable of two-way communication with computing device 628. For example, in accordance with such an embodiment, each of the patient-wearable devices is capable of sending sensor data 638 to computing device 628 and is also capable of receiving device control information 640 therefrom. Different ones of the patient-wearable devices may utilize different frequency bands and/or different time periods to communicate with computing device 628 so as to avoid interference.

Device control information 640 may comprise any information sent by computing device 628 to control the operation of any one of the patient-wearable devices. For example, in an embodiment, the patient-wearable devices may be designed to operate in a sleep mode (e.g., low power consumption mode) to preserve power of a battery included therein, thereby enabling the device operate over a longer period of time. For example, during sleep mode, the generation and/or transmission of sensor data may be disabled. In further accordance with such an embodiment, computing device 628 may send a “sleep” command to any one of the patient-wearable devices to place the device into sleep mode and also send a “wake” command to the device to cause it to resume generating and transmitting sensor data. Such a feature may be particularly useful for codes, which can last from 30 to 45 minutes, to cause pulse checks to occur every two to three minutes with each active pulse check lasting up to 30 seconds. One or more patient-wearable devices may be wakened from sleep mode to perform the pulse check, and then placed back into sleep mode when the pulse check is finished.

In another embodiment, device control information 640 may include information that can assist with system function monitoring. For example, device control information 640 may include an error message that indicates that there is an error in a data stream received from a patient-wearable device, a lack of a data stream altogether, or some other issue, such that the device can take some action to rectify the issue or notify a user thereof. For example, in an embodiment in which the patient-wearable device includes one or more LED indicators, the receipt of such an error message may cause the patient-wearable device to utilize such LED indicator(s) to signal that an error condition exists.

In certain embodiments, the processing of sensor data, detection of a pulse condition, and generation of an indicator thereof may all be performed by a single patient-wearable device without the need for an external computer. In other embodiments, each patient-wearable device may include the capacity for processing sensor data as well as the ability to perform multi-way communication with one or more external computers or devices. The external computers or devices may themselves be other patient-wearable devices. In such a case, any computation necessary to process sensor data and/or determine a pulse condition may take place in a distributed manner, occurring across all the patient-wearable devices, with communication happening between them, and one or multiple ones of the patient-wearable devices may present an indication of a determined pulse condition. The determination of which patient-wearable devices perform which functions may be negotiated dynamically amongst the patient-wearable devices. Alternatively, a single patient-wearable device may be determined to be a master or primary device and the other patient-wearable devices may be determined to be slave or secondary devices, and the master/primary device may determine which slave/secondary devices perform which functions.

In a further embodiment, a particular one of the patient-wearable devices (e.g., a primary or master device) attached to a patient may collect sensor data from other patient-wearable devices attached to the patient via a short-range wireless communication protocol (e.g., Bluetooth®) or even through wired connections thereto. The particular one of the patient-wearable devices may then transmit the collected sensor data along with its own sensor data to an external computer (e.g., computing device 628) using a long-range wireless communication protocol (e.g., WiFi®), and the external computer can process the sensor data to detect one or more pulse conditions and generate indicator(s) thereof.

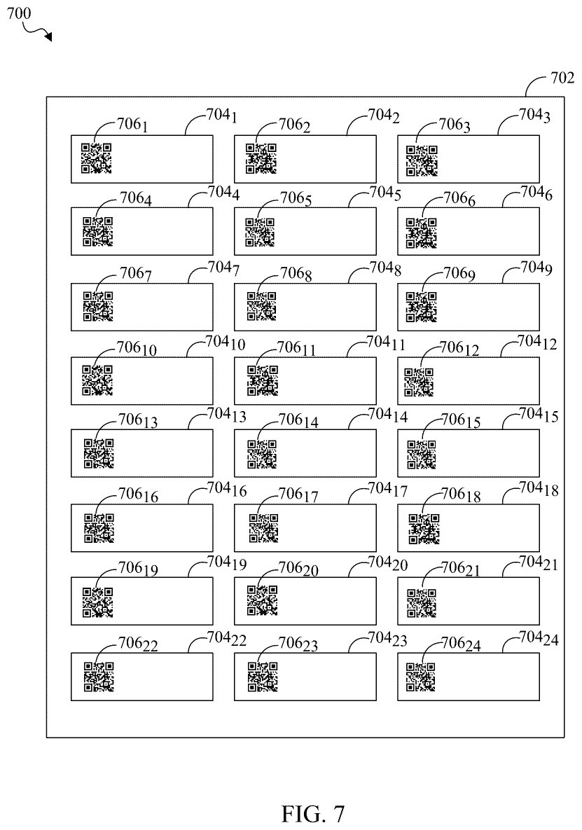

Prior to application to a patient, patient-wearable devices (such as device 100) may be stored adhesive-side down on a suitable substrate, such as a sheet or roll. For example, FIG. 7 depicts a sheet 700 of twenty-four patient-wearable devices 704₁-704₂₄ in accordance with an embodiment. It will be readily understood that a different number of devices may be accommodated on a sheet depending on the size and shape of the sheet and of the respective devices. Sheet 700 comprises a backing sheet 702. Devices 704₁-704₂₄ may be secured adhesive-side down to backing sheet 702 and can be removed therefrom as desired for application to the body of a patient. Backing sheet 702 may be formed from plastic, from paper coated with a suitable release agent (e.g., silicone, polyethylene terephthalate (PET) plastic resin, or polypropylene plastic resin), or from any other materials suitable for facilitating easy removal of devices 704₁-704₂₄ therefrom with the corresponding adhesive layer substantially intact.

As shown in FIG. 7, each device 704₁-704₂₄ has a corresponding (e.g., unique) identifier 706₁-706₂₄ printed thereon. In the example of FIG. 7, the identifier comprises a barcode but any type of identifier may be used. Such identifier can be scanned prior to, during, or after use of a patient-wearable device to associate it with a particular patient. If the patient-wearable device includes an internal battery, a battery preservation mechanism can be utilized while the device sits dormant. For example, as a patient-wearable device is removed from its respective sheet, roll, or other storage location, a small tab can be pulled on the device that allows the battery to make contact with the rest of the device, powering it on. In another embodiment, the act of separating the patient-wearable device from the sheet or roll or scanning the corresponding identifier has the effect of powering on the device. Still other methods and mechanisms for powering on a patient-wearable device may be used.

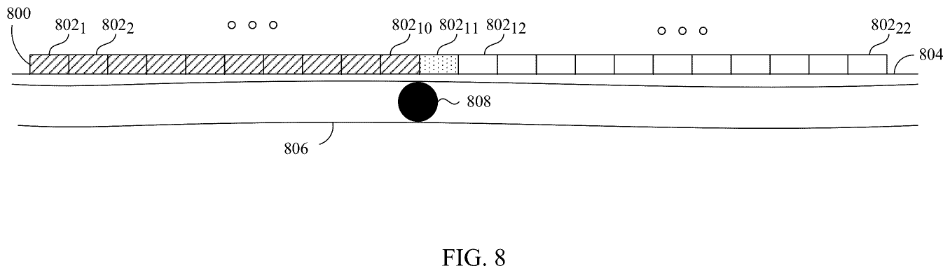

In certain embodiments, a patient-wearable device may provide a comparative grade of pulse strength along its length or on its structure. For example, FIG. 8 illustrates a side view of a patient-wearable device 800 in accordance with such an embodiment. As shown in FIG. 8, device 800 is applied to the skin 804 of a patient at a location along an artery 806 where an arterial occlusion 808 exists. In the example of FIG. 8, device 800 comprises twenty-two sensing modules 802₁-802₂₂, although a smaller or greater number of modules may be included as deemed necessary or desirable. Each of the sensing modules 802₁-802₂₂ may include one or more sensors to generate sensor data, a processing unit (e.g., microcontroller, microprocessor, DSP and/or ASIC) to process the sensor data and determine a pulse strength therefrom, and a visual indicator (e.g., LED indicator or the like) of the determined pulse strength. However, in an alternate embodiment, a sensing module may utilize a single sensor and/or processing unit for driving multiple visual indicators across its length. For example, a detection area of a sensor may be such that it can drive multiple visual indicators along its length to indicate pressure change across the span from beginning to end of the sensor.

In the example shown in FIG. 8, the visual indicators of sensing modules 802₁₂-802₂₂ on device 800 proximal to occlusion 808 signify detection of a pulse (e.g., by showing a relatively bright illumination or by illuminating a green light) in the patient, while the visual indicators of sensing modules 802₁-802₁₀ associated with the distal aspect of artery 806 signify no pulse (e.g., by showing no illumination or by illuminating a red light). The transition between the two suggests the location over the occlusion, which is further be signified by a visual indicator of sensing module 802₁₁ (e.g., by showing an intermediate level of illumination or by illuminating a yellow light). This embodiment of FIG. 8 thus allows for obstruction location detection by a medical provider.

As discussed above in reference to FIG. 1, patient-wearable device 100 may comprise an adhesive layer 112 that enables device 100 to be affixed to the body (e.g., the skin) of a patient. Such adhesive layer may comprise, for example and without limitation, a ready-for-use adhesive pad or film or a replaceable adhesive pad or film that may be attached to the skin or attached to device 100 prior to application to a patient. In the embodiment of FIG. 1, adhesive layer 112 is attached to the bottom of device 100, which may be contacting PCB 102, to the bottom of a base sheet that includes PCB 102 or to which PCB 102 itself is connected, or to some other component of device 100 that can contact the body of a patient. However, this example is not intended to be limiting. An adhesive layer may be connected to a patient-wearable device in a variety of different ways to support affixing the device to the patient.

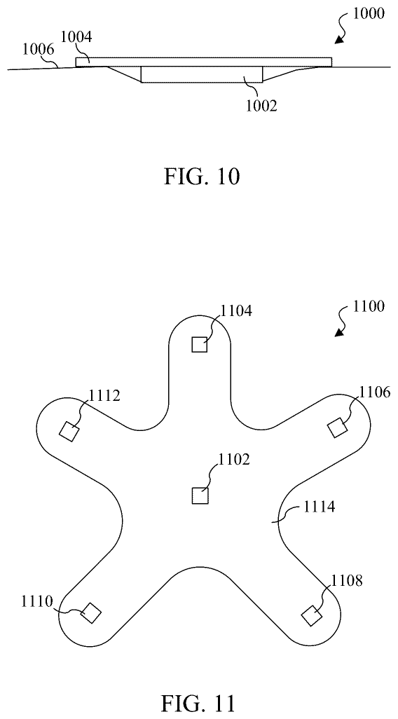

By way of example, FIG. 10 depicts a side view of a patient-wearable device 1000 that includes a housing 1002 and an adhesive layer 1004 that is attached to and extends laterally outward from a top side of housing 1002. With respect to the embodiment of FIG. 10, the “top side” of housing 1002 is the side of housing 1002 that faces away from the body of the patient when device 1000 is attached thereto, while the “bottom side” of housing 1002 is the side of housing 1002 that faces toward the body of the patient when device 1000 is attached thereto. Housing 1002 is intended to represent any structure that houses, encapsulates, covers or supports a PCB as well as electronics and an antenna that are disposed on such PCB (such as PCB 102, electronics 106 and antenna 108 as described above in reference to FIG. 1) and that operate in a manner described above to facilitate the detection of a pulse condition of a patient.

As further shown in FIG. 10, at least a portion of adhesive layer 1004 that extends outward from the top side of housing 1002 may be affixed to a body 1006 of a patient in such a manner that housing 1002, or some portion of device 1000 contained in housing 1002, is pushed, to at least some degree, into the surface of the skin of the patient while device 1000 is attached thereto. Pushing housing 1002 into the surface of the skin of the patient may have a beneficial effect, for example, by bringing the sensors of device 1000 closer to the blood vessels of the patient or by physically separating certain sensors from sources of stimuli, thereby enhancing the ability of those sensors to detect a pulse or subpulse of the patient. Also, because housing 1002 may essentially be sealed between adhesive layer 1004 and the skin of the patient when device 1000 is attached thereto, housing 1002 may be rendered less susceptible to being jostled or moved due to external forces (e.g., someone or something brushing up against the body of the patient). Likewise, because housing 1002 may essentially be sealed between adhesive layer 1004 and the skin of the patient when device 1000 is attached thereto, sensors present on or within housing 1002 may be rendered less susceptible to ambient stimuli in the environment around the patient if adhesive layer 1004 has stimulus-dampening (e.g., in the case of an acoustic sensor, sound-absorptive and/or sound-reflective) characteristics. That is to say, when adhesive layer 1004 has certain stimuli-dampening characteristics, the encapsulation of such sensors between adhesive layer 1004 and body 1006 of the patient may help to isolate such sensors from external stimuli sources, or at least mitigate the impact of such external stimuli sources on the sensors.

Although the foregoing describes the use of adhesive layer 1004 to push housing 1002 into the surface of the skin of the patient, it is also noted that the attachment of adhesive layer 1004 to the surface of the skin of the patient may additionally or alternatively cause the skin that surrounds housing 1002 to be pulled upward, achieving a similar encapsulating effect. Furthermore, factors other than the use of adhesive layer 1004 may cause the patient-wearable device to be pushed into the surface of the skin of the patient, such as the weight or firmness of device 1000 itself. For example, depending upon the choice of materials, the weight of an overlay or housing that covers or encapsulates the PCB, electronics, and antenna of the device may have the effect of causing the device to be pushed into the surface of the skin of the patient when the patient-wearable device is attached thereto.

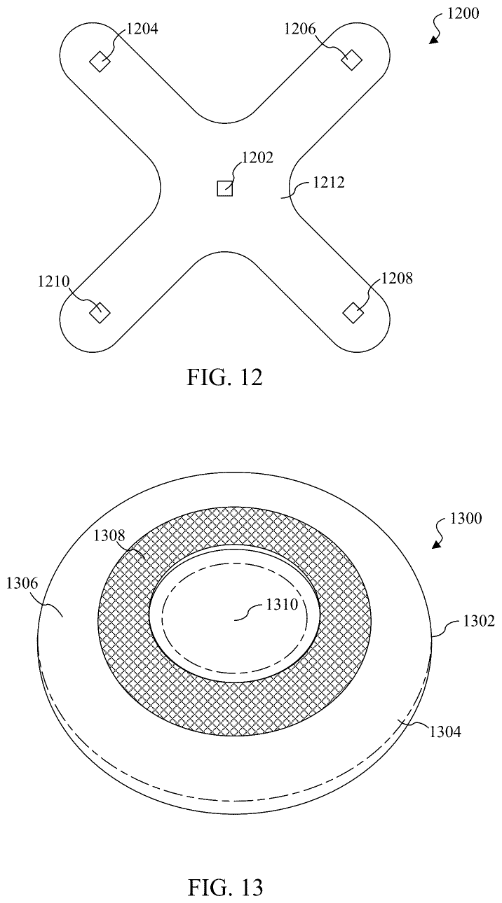

An adhesive layer that is used to affix a patient-wearable device to a body of a patient may have any of a variety of shapes and be of any of a variety of sizes appropriate for a particular location on the body or for a specific sensor combination. For example, FIG. 11 depicts a patient wearable device 1100 that includes a star-shaped adhesive layer 1114, while FIG. 12 depicts a patient wearable device 1200 that includes an X-shaped adhesive layer 1212. Generally speaking, utilizing an adhesive layer with greater surface area and/or multiple arms may increase the size and/or the number of the points of contact between the adhesive layer and the body of the patient and/or facilitate a desired distance between sensors within device 100, thereby improving the ability of device 100 to achieve its intended function. Furthermore, the shape and size of the adhesive layer may be selected based on suitability for attachment to a particular body part of the patient. For example, a star shape or X shape may be particularly suitable for attachment to a patient's chest (e.g., over the patient's heart and ribs), while a band, cuff, elongated rectangle, square, circle, or irregular shape may be particularly suitable for attachment to a patient's limb.

Although the patient-wearable devices of FIGS. 11 and 12 each include an adhesive layer that comprises a plurality of “arms” of similar length, this need not be the case and different arms of each device's adhesive layer may be of different lengths.

Furthermore, in certain embodiments, a plurality of sensing modules may be attached to different portions of a single adhesive layer, such that the plurality of sensing modules can easily be attached to the body of a patient through the application of the single adhesive layer. For example, as shown in FIG. 11, patient-wearable device 1100 comprises six sensing modules 1102, 1104, 1106, 1108, 1110 and 1112, each of which is attached to a different portion of adhesive layer 1114 and, as shown in FIG. 12, patient-wearable device 1200 comprises five sensing modules 1202, 1204, 1206, 1208 and 1210, each of which is attached to a different portion of adhesive layer 1212. Each of the aforementioned sensing modules may include one or more sensors to generate sensor data and a processing unit (e.g., microcontroller, microprocessor, DSP and/or ASIC) to process the sensor data and determine a pulse strength therefrom. Each of the aforementioned sensing modules may also include an indicator (e.g., a visual indicator such as an LED) that indicates the determined pulse strength and/or a wired or wireless interface for communicating the determined pulse strength within or between devices.

The plurality of sensing modules of patient-wearable device 1100 of FIG. 11 or the plurality of sensing modules of patient-wearable device 1200 of FIG. 12 may operate in a like manner to the plurality of patient-wearable devices of system 600 shown in FIG. 6, as previously described. For example, each of the sensing modules may be capable of transmitting sensor data to a computing device and/or receiving device control information from such computing device via a wired or wireless connection. Likewise, the plurality of sensing modules may operate to concurrently detect pulse conditions at different body locations which, as discussed above in reference to FIG. 6, may be particularly beneficial in situations of cardiac arrest, other critical conditions for which rapid pulse detection (or lack thereof) is pivotal, and for monitoring following certain invasive procedures. In certain embodiments, the different sensing modules may be capable of communicating with each other, which may facilitate data sharing amongst the sensing modules as well as distributed processing scenarios.

Different ones of the sensing modules of patient-wearable device 1100 of FIG. 11 or different ones of the sensing modules of patient-wearable device 1200 of FIG. 12 may possess the same type of sensor or may possess different types of sensors. For example, sensing module 1202 of patient-wearable device 1200 of FIG. 12 may include an ECG sensor while the other sensing modules of patient-wearable device 1200 do not. In further accordance with such an example, when patient-wearable device 1200 is attached to the body of a patient, it may be attached in such a manner that sensing module 1202 (or any of sensing modules 1204, 1206, 1208 or 1210) is placed at or near the Point of Maximal Impulse (PMI) on the chest of the patient. This will have the effect of improving the performance of the ECG sensor. As discussed elsewhere herein, the ECG data collected by sensing module 1202 may then be shared with and used by any one or more of the sensing modules or an external computing device to filter data collected by other types of sensors, such as an acoustic sensor or an IMU. For example, sensor data obtained by an acoustic sensor or an IMU may be filtered in at least a time domain by accounting for data that deviates from an electrical signal detected using the aforementioned ECG sensor.

In certain embodiments of patient-wearable device 1100 and patient-wearable device 1200, the adhesive layer is attached to the bottom of the sensing modules such that the adhesive layer will be between the sensing modules and the body of the patient when the device is connected to the body of the patient. In alternate embodiments of patient-wearable device 1100 and patient-wearable device 1200, the adhesive layer is attached to the top of the sensing modules such that the sensing modules will be between the adhesive layer and the body of the patient when the device is connected to the body of the patient. In such an embodiment, the attachment between the adhesive layer and the skin of the patient may have the effect of pushing the sensing modules into the skin of the patient, which may have the beneficial effect of bringing the sensors closer to the blood vessels of the patient and also, when the adhesive layer has stimuli-barring characteristics, isolating any sensors of the sensing modules from external stimuli, as discussed above in reference to the embodiment of FIG. 10. In still further embodiments of patient-wearable device 1100 and patient-wearable device 1200, the adhesive layer may be attached to the bottom of some of the sensing modules and to the top of other ones of the sensing modules such that some of the sensing modules will be above the adhesive layer and other sensing modules will be below the adhesive layer and in contact with the body of the patient when the device is connected to the body of the patient.

Although the embodiments of FIGS. 11 and 12 each have one sensing module at the center of the adhesive layer and one sensing module at or near the end of each arm emanating from the center of the adhesive layer, this need not be the case and the sensing modules may instead be placed at any location with respect to the adhesive layer. For example, in alternative embodiments, a cluster of sensing modules may be located in central part of the adhesive layer and the arms of the adhesive layer may be used only to connect the device to the body of the patient. In other alternative implementations, for example, patient-wearable devices 1100 or 1200 (or any other embodiment described herein) may contain as few as a single sensor module located thereon.

FIG. 13 depicts a top and side perspective view of a patient-wearable device 1300 for detecting a pulse condition of a patient in accordance with a further embodiment. As shown in FIG. 13, device 1300 comprises a housing 1302 that houses, encapsulates, covers or supports various components of device 1300 that will be described in more detail below. In embodiments, housing 1302 is formed from a material suitable for application on the skin of a patient such as, for example, silicone or a silicone-like material such as thermoplastic elastomer (TPE), thermoplastic rubber (TPR), or thermoplastic polyurethane (TPU), although other materials may be used.

As further shown in FIG. 13, housing 1302 comprises a top side 1304 that itself includes an outer circumferential portion 1306, an inner circumferential portion 1308, and a central depression 1310. Central depression 1310 may be formed from a same or different material than the rest of housing 1302. For example, central depression 1310 may be formed from a transparent or quasi-transparent material (e.g., transparent or translucent silicone or plastic) such that certain visual indicators (e.g., LEDs) disposed within housing 1302 will be visible through central depression 1310. Central depression 1310 may be molded or otherwise permanently affixed to top side 1304 of housing 1302. Alternatively, central depression 1310 may comprise a removable cap (e.g., a press fit cap, a screw on cap, a hinged cap, or the like) that can be connected to and disconnected from top side 1304 of housing 1302. In still further embodiments, central depression 1310 may simply comprise an opening in the center of top side 1304 of housing 1302 via which various internal components of device 1300 may be visible or accessible, or central depression 1310 may be omitted entirely.

Inner circumferential portion 1308 may have a rough or uneven surface that makes device 1300 easier to grip and hold onto while outer circumferential portion 1306 may have a smoother surface. This may be particularly helpful when housing 1302 is formed from silicone, which can be slippery, and/or if device 100 becomes wet due to the presence of blood or other liquids. It will be appreciated that different surface qualities of housing 1302 may also cause diffuse refraction of light from light sources from within device 100, which can make visual indicators visible within depression 1310 or elsewhere on housing 1302 easier to see. In alternate embodiments, additional or different portions of top side 1304 of housing 1302 may be textured. Likewise, some or all of a circumferential edge or a bottom of housing 1302 may be textured depending upon the implementation. It will be further appreciated that while housing 1302 of FIG. 13 comprises a top side 1304 that is dome-shaped, housing 1302 and top side 1304 may be flatter or more rounded in shape to meet the desired configuration for an application or location on the body of the patient.

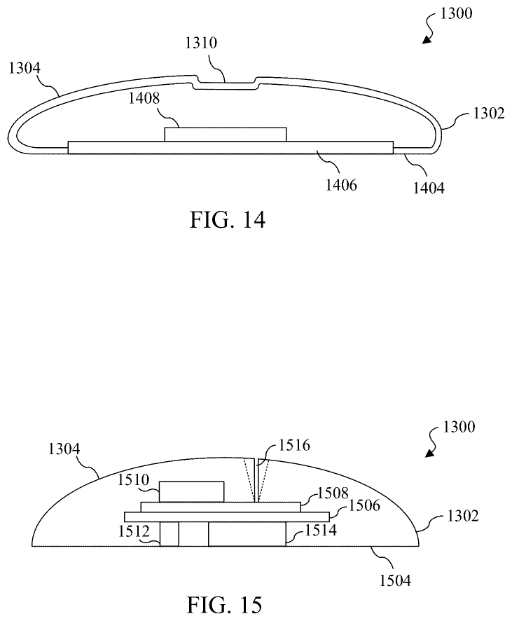

FIG. 14 depicts a cross-sectional side view of device 1300 in accordance with one embodiment. In the embodiment of FIG. 14, device 1300 includes a PCB 1406 (e.g., a single-sided PCB) and a bottom side 1404 of housing 1302 comprises a bottom side of PCB 1406. Furthermore, various electronic components 1408 are connected to a top side of PCB 1406. These electronic components 1408 may include components described herein (e.g., components described above in reference to FIG. 4), such as one or more sensors, one or more passive electronic components, a battery, a microcontroller, and an antenna, and such electronic components 1408 may operate in a manner described above to facilitate the detection of a pulse condition of a patient.

In an embodiment, housing 1302 of device 1300 may be formed by depositing or molding a material (e.g., silicone) on top of or around PCB 1406 and electronic components 1408. Housing 1302 may thus be substantially solid throughout. In an alternative implementation, an internal cavity may be created between the top of PCB 1406/electronics 1408 and an inner side of top 1304 of housing 1302 and such cavity may comprise a material that alters the performance of the sensors.

In the embodiment shown in FIG. 14, the material of housing 1302 that is disposed above electronic components 1408 may act as an insulating layer with respect to various sensors included in electronic components 1408. For example, in an embodiment in which electronic components 1408 include an IMU, the material of housing 1302 may insulate the IMU from forces that originate outside of a patient's body that might otherwise be sensed, or more strongly sensed, by the IMU. As another example, in an embodiment in which electronic components 1408 include an acoustic sensor, the material of housing 1302 may insulate the acoustic sensor from sound waves that emanate from sources outside of a patient's body that might otherwise be sensed, or more strongly sensed, by the acoustic sensor. For example, the material of housing 1302 may have sound-barring (e.g., sound-reflecting and/or sound-absorbing qualities) characteristics that may help insulate the acoustic sensor from external sources of noise.

FIG. 15 depicts a cross-sectional side view of device 1300 in accordance with another embodiment. In the embodiment shown in FIG. 15, device 1300 includes a double-sided PCB 1506 having a top side to which electronic components 1508 and 1510 are attached and a bottom side to which electronic components 1512 and 1514 are attached. The electronic components collectively attached to either side of PCB 1506 may include components described herein (e.g., components described above in reference to FIG. 4), such as one or more sensors, one or more passive electronic components, a battery, a microcontroller, and an antenna, and such components may operate in a manner described above to facilitate the detection of a pulse condition of a patient.

In an embodiment, housing 1302 of device 1300 may be formed by depositing or molding a material (e.g., silicone) around PCB 1406 and electronic components 1508, 1510, 1512 and 1514, such that body-facing sides of electronic components 1512 and 1514 are substantially flush with and essentially form a part of a bottom 1504 of housing 1302. In an implementation in which electronic components 1512 and 1514 include sensors (e.g., IMUs or acoustic sensors), such a design may beneficially position such sensors as close to the body of the patient as possible and minimize or remove any structural barriers (e.g., PCB 1506 or a portion of housing 1302) from between such sensors and the body of the patient, all of which may improve the ability of such sensors to detect a pulse condition of the patient.

As further shown in FIG. 15, housing 1302 may comprise or form a channel 1516 that extends from electronics components 1508 on the top side of PCB 1506 to top side 1304 of housing 1302. When the embodiment of FIG. 15 is viewed from above, channel 1516 may appear as a hole or a cone. Electronic components 1508 located at or near the bottom of channel 1516 may comprise a first acoustic sensor (e.g., a PCB-based low-powered MEMs acoustic sensor). The presence of channel 1516 may enable such first acoustic sensor to sense, or more strongly and/or accurately sense, sound waves generated by sources in an environment around device 1300 and external to the body of the patient. By operating in this manner, the first acoustic sensor can identify one or more background noise signals. Such background noise signal(s) may then be used to filter or correct audio signals captured by one or more other acoustic sensors within device 1300 and/or within other devices that are directly or indirectly communicatively connected to device 1300.

For example, the embodiment of FIG. 15 may further include one or more acoustic sensors connected to PCB 1506 (e.g., as part of electronic components 1512 or 1514) that are positioned to receive stimuli from the body of the patient when device 1300 is connected thereto. In accordance with such an embodiment, PCB 1506 and housing 1302 may act as acoustic barriers that help isolate individual acoustic sensor(s) from one another and from other sources of stimuli. Thus, such additional acoustic sensor(s) may obtain a cleaner (e.g., more noise-free) audio signal for detecting a pulse condition of the patient. Moreover, to the extent audio data captured by any acoustic sensor includes noise components different from the other(s) by type or intensity, such noise components may be partially or substantially removed, filtered or otherwise accounted for as desired.

Although not shown in FIG. 15, in certain embodiments in which a second acoustic sensor is not flush with bottom 1504 of housing 1302 of device 1300, a second channel may be formed between the second acoustic sensor and bottom 1504 of housing 1302 to enhance the ability of such second sensor to detect a pulse or subpulse of the patient. Furthermore, although the above description mentions the possibility of a single acoustic sensor on either side of PCB 1506, it should be understood that any number of acoustic sensors may be placed on either side of PCB 1506. For example, two or more acoustic sensors may be disposed on the top side of PCB 1506 and used to capture ambient/background noise while two or more acoustic sensors may be disposed on the bottom side of PCB 1506 and used to detect a pulse or subpulse. Additionally, any number of channels may be formed in top 1304 of housing 1302 or bottom 1504 of housing 1302 to improve the performance of such acoustic sensors.

Also, although only a single PCB 1506 is shown in FIG. 15, it is to be understood that any number of PCBs may be present in device 1300. For example, two PCBs may be present (e.g., one affixed at a certain distance atop the other) to provide a four-layer system. The number of PCBs used may be determined based on such factors as the number and size of electronic components to be included in device 1300, the size and shape of device 1300, or the like.

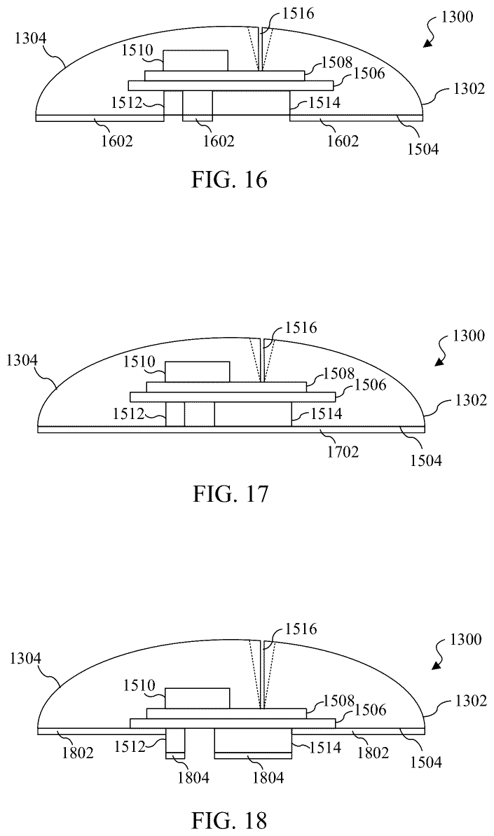

FIG. 16 depicts a cross-sectional side view of a further embodiment of device 1300 that is similar to the embodiment shown in FIG. 15, except that an adhesive layer 1602 has been disposed on or attached to bottom side 1504 of housing 1302. Adhesive layer 1602 may be used to facilitate attachment of device 1300 to the body (e.g., the skin) of a patient and may be implemented in any manner previously described herein, including in any manner previously described with reference to adhesive layer 112 of device 100. In the embodiment of FIG. 16, adhesive layer 1602 only partially covers bottom side 1504 of housing 1302. In particular, adhesive layer 1602 does not cover the portions of bottom side 1504 of housing 1302 beneath electronics components 1512 and 1514. Such a design may be deemed desirable if adhesive layer 1602 has one or more characteristics that may impede or degrade the functioning of electronics components 1512 and 1514. For example, such a design may be deemed desirable if the presence of adhesive layer 1602 negatively impacts the performance of (e.g., attenuates a signal sensed by) an acoustic sensor or IMU within electronic components 1512 and 1514.

FIG. 17 depicts a cross-sectional side view of a further embodiment of device 1300 that is similar to the embodiment shown in FIG. 15, except that an adhesive layer 1702 has been disposed on or attached to bottom side 1504 of housing 1302. Adhesive layer 1702 may be used to facilitate attachment of device 1300 to the body (e.g., the skin) of a patient and may be implemented in any manner previously described herein, including in any manner previously described with reference to adhesive layer 112 of device 100. In the embodiment of FIG. 17, adhesive layer 1702 covers all or substantially all of bottom side 1504 of housing 1302. Such a design may be deemed desirable if adhesive layer 1602 does not have any characteristics that may impede or degrade the functioning of electronics components 1512 and 1514, or if adhesive layer 1602 has one or more characteristics that may improve the functioning of electronics components 1512 and 1514. For example, such a design may be deemed desirable if the presence of adhesive layer 1702 improves the performance of (e.g., amplifies a signal sensed by) an acoustic sensor or IMU within electronic components 1512 and 1514.

FIG. 18 depicts a cross-sectional side view of a further embodiment of device 1300 that is similar to the embodiment shown in FIG. 15, except that in this embodiment the internal components have been shifted downward, such that electronic components 1512 and 1514 protrude outward from bottom side 1504 of housing 1302. A first adhesive layer 1802 may be disposed on or attached to bottom side 1504 of housing 1302, while a second adhesive layer 1804 may optionally be disposed on or attached to electronic components 1512 and 1514. These adhesive layers may be used to facilitate attachment of device 1300 to the body (e.g., the skin) of a patient and may be implemented in any manner previously described herein, including in any manner previously described with reference to adhesive layer 112 of device 100. When the embodiment of device 1300 shown in FIG. 18 is attached to the body of a patient, the particular configuration of components will cause electronic components 1512 and 1514 to be pushed deeper into the skin of the patient. When electronic components 1512 and 1514 comprise sensors, this may have the beneficial effect of bringing the sensors closer to the blood vessels of the patient, thereby enhancing the ability of those sensors to detect a pulse or subpulse of the patient.

In embodiments discussed above with respect to FIGS. 6, 11 and 12, different patient-wearable devices or different sensing modules may be attached to different locations on a body of a patient and concurrently utilized to detect a pulse condition of the patient. As also noted above, the different patient-wearable devices or different sensing modules may comprise different sensors. For example, a first patient-wearable device or sensing module attached to a first location on a body of a patient may include a first type of sensor but not a second type of sensor, whereas a second patient-wearable device or sensing module attached to a second location on the body of the patient may include the second type of sensor but not the first type of sensor. Possible types of sensors that may be included within a patient-wearable device or sensing module may include but are not limited to: an accelerometer, a gyroscope, a magnetometer, an IMU (which may itself comprise one or more of an accelerometer, a gyroscope or a magnetometer), an acoustic sensor, an ECG sensor, a carbon dioxide (CO₂) sensor, a blood oxygen (SpO₂) sensor, or a sensor that is capable of detecting one or more of blood pressure, blood sugar, blood pH, body temperature, respiratory rate, blood flow rate, magnetic fields, or the like.

In certain embodiments, each of a plurality of patient-wearable devices or sensing modules may include the same plurality of sensor types, but individual ones of the devices/modules may be controlled to activate a different subset of the plurality of sensor types. That is to say, each device/module may be individually controlled to activate or deactivate different ones of the plurality of sensor types included therein. By way of example, assume that each of the plurality of devices/modules includes an IMU, an acoustic sensor and an ECG sensor. Furthermore, assume that a respective one of these devices/modules have been attached to the following locations on the body of the patient: over the heart, on the right wrist, on the left wrist, on the right ankle, and on the left ankle. In accordance with such a scenario, the device/module over the heart may be controlled such that only the ECG sensor is active and the other sensors, such as IMUs or acoustic sensors, are inactive, the devices/modules on the wrist may be controlled such that various combinations of IMUs and/or acoustic sensors are active and the ECG sensor is inactive, and the devices/modules on the ankles may be controlled such that the same or different combinations of IMUs and/or acoustic sensors as in the devices/modules on the wrists are active and the ECG sensor is inactive. Of course, this is only one example and a wide variety of different sensor types and selective activation/deactivation control schemes may be used.

Control of a patient-wearable device or sensing module for the purposes of selectively activating or deactivating one or more sensors included therein may be achieved in a variety of ways. For example, if the patient-wearable device or sensing module comprises a wired or wireless interface, then the device/module can receive commands from an external device (e.g., from a computing device such as computing device 628 of FIG. 6 or from another patient-wearable device or sensing module) and then execute such commands to cause one or more sensors to be activated or deactivated. As another example, the patient-wearable device or sensing module may comprise one or more mechanical user interface elements (e.g., buttons, toggles, switches, or the like) that a user may interact with to selectively activate or deactivate particular sensors on the device/module. In an alternative embodiment, individual patient-wearable devices or sensing modules may be oriented (by user input, location-specific device selection, barcode, or some other means) according to its respective location on the body of the patient, such as the carotid artery, which may dictate which sensor combination is utilized in a predefined manner. Such location-specific orientation may, for example, aid data processing and filtering of incoming location-specific stimuli.

An approach in which all of the patient-wearable devices or sensing modules include the same complement of sensors but that allows for sensors to be activated or deactivated on a per device/module basis can be beneficial in that only one version of the device/module need be produced and each device/module can be applied to any location on the body of the patient without regard for which sensors are on board. In contrast, in accordance with an alternate approach in which different patient-wearable devices or sensing modules actually include different sensors, multiple versions of the devices/modules must be produced and certain versions of the devices/modules may be targeted to particular body locations where certain sensors are desired or most effective. Benefits may be achieved from either approach and may include one or more of the following: case of use; cheaper manufacturing costs per device/module; superior data collection due to less interference between onboard sensors; more optimal placing of sensors; reduced power consumption per device/module; and increased flexibility in trace geometry and/or overall PCB design.

It is noted that some embodiments may combine the foregoing approaches. That is to say, in some embodiments, a plurality of patient-wearable devices or sensing modules may all share a common set of sensor types that may be selectively activated/deactivated but they may also include different sensor types as well.

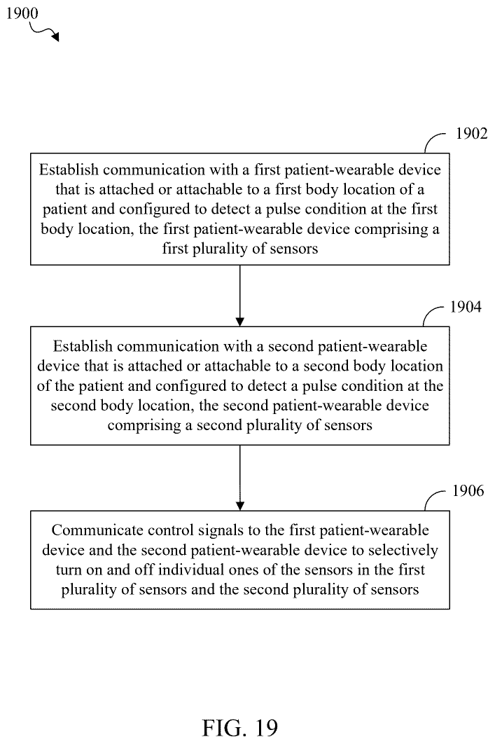

FIG. 19 depicts a flowchart 1900 of a method for selectively activating/deactivating sensors of a plurality of patient-wearable devices that are attached or attachable to different locations on a body of a patient, in accordance with an embodiment. The method of FIG. 19 may be implemented, for example, by processing unit 634 of system 600 which, as discussed above in reference to FIG. 6, may be communicatively connected to multiple patient-wearable devices 602, 604, 606, 608, 610, 612, 614, 616, 618, 620, 622, 624, 626 that are concurrently attached to different locations on the body of patient 650. However, this example is not intended to be limiting and the method of flowchart 1900 may be implemented by any processing unit that is capable of connecting to and communicating with multiple patient-wearable devices, wherein each of the patient-wearable devices comprises a plurality of sensors. Such processing unit may, for example, be separate from and communicatively connected to each of the patient-wearable devices (as is the case in system 600 of FIG. 6), or such processing unit may form part of one or more of the patient-wearable devices. Still further, the steps of flowchart 1900 may be performed in a distributed manner by multiple processing units.

Furthermore, although the method of flowchart 1900 refers to a plurality of patient-wearable devices that each comprise a plurality of sensors, it is to be understood that the method can also be applied to a plurality of sensing modules (e.g., the plurality of sensing modules present in the respective embodiments of FIG. 8, FIG. 11 or FIG. 12) when such sensing modules comprise a plurality of sensors.

As shown in FIG. 19, the method of flowchart 1900 begins at step 1902 in which the processing unit establishes communication with a first patient-wearable device that is attached or attachable to a first body location of a patient and configured to detect a pulse condition at the first body location, wherein the first patient-wearable device comprises a first plurality of sensors. For example, the first patient-wearable device may a first one of patient-wearable devices 602, 604, 606, 608, 610, 612, 614, 616, 618, 620, 622, 624, 626 which is attached or attachable to a particular location on the body of patient 650 as discussed above in reference to FIG. 6. The first patient-wearable device may be configured to detect a subpulse condition at the respective body location. The processing unit may establish communication with the first patient-wearable device using one or more of a wireless communication link (e.g., a Bluetooth®, Wi-Fi® or other RF communication link) or a wired communication link (e.g., a USB or other wired communication link).

At step 1904, the processing unit establishes communication with a second patient-wearable device that is attached or attachable to a second body location of the patient and configured to detect a pulse condition at the second body location, wherein the second patient-wearable device comprises a second plurality of sensors. For example, the second patient-wearable device may be a second one of patient-wearable devices 602, 604, 606, 608, 610, 612, 614, 616, 618, 620, 622, 624, 626 which is attached or attachable to a particular location on the body of patient 650 as discussed above in reference to FIG. 6. The second patient-wearable device may also be configured to detect a subpulse condition at the respective body location. The processing unit may establish communication with the second patient-wearable device using one or more of a wireless communication link (e.g., a Bluetooth®, Wi-Fi® or other RF communication link) or a wired communication link (e.g., a USB or other wired communication link).

In certain embodiments, the first plurality of sensors and the second plurality of sensors include a same variety of sensor types. For example, the first plurality of sensors may include at least a first IMU and a first acoustic sensor and the second plurality of sensors may include at least a second IMU and a second acoustic sensor. In further accordance with such an embodiment, the first plurality of sensors and the second plurality of sensors may further include: a first ECG sensor and a second ECG sensor, respectively; a first carbon dioxide sensor and a second carbon dioxide sensor, respectively; and/or a first blood oxygen sensor and a second blood oxygen sensor, respectively. However, these are only non-limiting examples, and the first plurality of sensors and the second plurality of sensors may both include still other sensor types.

At step 1906, the processing unit communicates control signals to the first patient-wearable device and the second patient-wearable device to selectively turn on and off individual ones of the sensors in the first plurality of sensors and the second plurality of sensors. The processing unit may selectively turn on or off the individual ones of the sensors in the first plurality of sensors and the second plurality of sensors based on one or more of: body locations to which the first patient-wearable device and the second patient-wearable device are or will be respectively attached; one or more factors associated with the environment of the patient; or a type of medical procedure that was or will be performed on the patient.

In a case where the selective activation/deactivation of sensors is performed based on the body locations of the first and second patient-wearable devices, the body location information for each patient-wearable device may be determined in a variety of ways. For example, a user may input information (e.g., to computing device 628) that associates an identifier of each patient-wearable device with a particular body location. In accordance with an embodiment such as that described above in reference to FIG. 7, in which a user may scan an identifier (e.g., barcode) of a patient-wearable device to associate the device with a particular patient, the user may also utilize the identifier to associate the device with a particular body location of the patient at the time of device activation or attachment. Such identifier may also include information about which sensors are included within the device. As another example, each patient-wearable device may communicate its own identifier and body location to the processing module via the aforementioned wireless or wired communication links. In such a scenario, each patient-wearable device may be pre-programmed to operate at a particular body location and transmits an indicator of the particular body location to the processing module. Alternatively, each patient-wearable device may be capable of sensing the body location to which it has been attached and transmits this information to the processing module. As yet another example, an external sensing device or system may determine the body locations of the patient-wearable determined by scanning the body of the patient to identify particular patient-wearable devices and the respective body locations to which they are attached and then pass such information to the processing module. However, these are only examples and still other techniques may be used to determine the body location information for each patient-wearable device.

In a case where the selective activation/deactivation of sensors is performed based on one or more factors associated with the environment of the patient, such factor(s) may be determined in a variety of ways. For example, a user may input information regarding such factor(s) (e.g., to computing device 628). As another example, the factor(s) may be determined by one or more of the patient-wearable devices (e.g., based on sensor data captured thereby) and communicated to the processing unit via the aforementioned wireless or wired communication links. As yet another example, an external sensing device or system may determine the factor(s) and then pass such information to the processing module. However, these are only examples and still other techniques may be used to determine the factor(s) associated with the environment of the patient.

In an embodiment, the processing module may operate to selectively turn on all sensors of a first type in the first plurality of sensors and turn off all sensors of the first type in the second plurality of sensors. For example, with reference to an example discussed above, the processing module may turn on an ECG sensor in the first plurality of sensors when the first patient-wearable device is attached to the patient's chest at or near the heart, while turning off the ECG sensor in the second plurality of sensors when the second patient-wearable device is attached to an extremity (e.g., wrist or ankle) of the patient.

In another embodiment, the processing module may operate to selectively turn on all sensors of a first type in the first plurality of sensors and the second plurality of sensors and turn off all sensors of a second type in the first plurality of sensors and the second plurality of sensors. For example, when the patient is in an environment with significant external noise, the processing module may turn off all acoustic sensors in the first plurality of sensors and the second plurality of sensors and turn on all IMUs in the first plurality of sensors and the second plurality of sensors. As another example, when the patient is moving or being moved (e.g., in a moving ambulance), the processing module may turn off all IMUs in the first plurality of sensors and the second plurality of sensors and turn on all acoustic sensors in the first plurality of sensors and the second plurality of sensors.

Various motivations may exist for providing different sensor types at different body locations. For example, having at least an accelerometer at different body locations may be deemed highly beneficial since different body parts may be moving differently. Thus, accelerometer data obtained from multiple locations can be compared and differences due to non-pulse-related motion can be identified and accounted for. Furthermore, ECG data (electrical signals) can be useful for interpreting overall collected data. As another example, in the case of detecting something like an obstructed artery, it may be useful to place devices in multiple locations to identify a potential blockage site and utilize a suite of sensors (e.g., IMU, acoustic sensor and ECG sensor) in those locations.

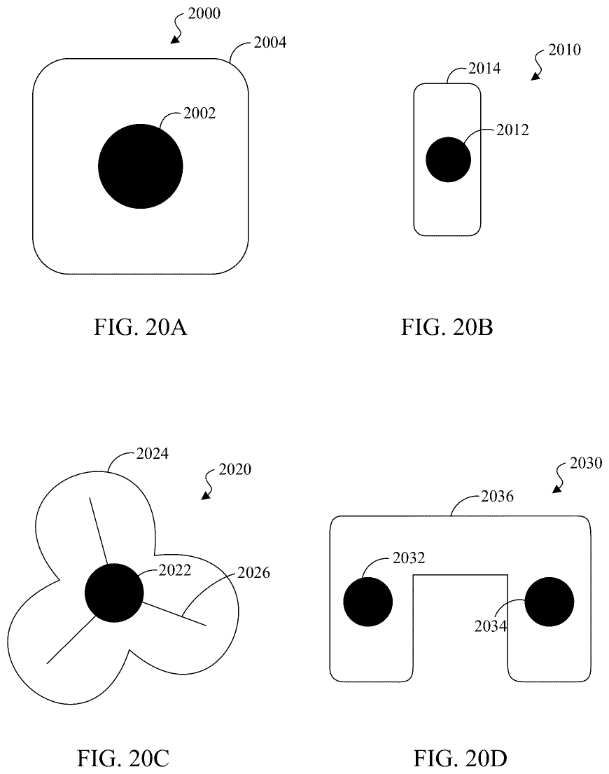

FIGS. 20A, 20B, 20C and 20D illustrate different patient-wearable device configurations that may be suitable for attachment to respective different locations on a body of a patient, in accordance with embodiments. Generally speaking, each of the patient-wearable device configurations includes one or more sensing modules attached to an adhesive layer, wherein the sensing module(s) may be implemented in a like manner to the sensing modules described above in reference to FIG. 8, 11 or 12, although the sensing module(s) may be implemented in other ways as well.

For example, FIG. 20A depicts a patient-wearable device 2000 that has a roughly square shape and that comprises a sensing module 2002 that is attached to an adhesive layer 2004. The configuration shown in FIG. 20A may be particularly suitable for attachment to a thigh of a patient, over or near a femoral artery thereof, or on an inside of a forearm of a patient, over or near a radial artery thereof.

FIG. 20B depicts a patient-wearable device 2010 that has a roughly rectangular shape and that comprises a sensing module 2012 that is attached to a an adhesive layer 2014. The configuration shown in FIG. 20B may be particularly suitable for attachment to a side of a neck of a patient, over a carotid artery thereof.

FIG. 20C depicts a patient-wearable device 2020 that has a roughly trefoil shape and that comprises a sensing module 2022 that is attached to an adhesive layer 2024. As further shown in FIG. 20C, patient-wearable device 2020 further comprises three leads or wires 2026 that emanate outward from sensing module 2022, each of which may terminate at a corresponding electrode, and each of which may be utilized to collect electrical signals for use by an ECG sensor included within sensing module 2022. In this embodiment, adhesive layer 2024 not only supports and attaches sensing module 2022 to the body of the patient, but it may also support and attach leads 2026 and the electrodes as well. The configuration shown in FIG. 20C may be particularly suitable for attachment to a chest of a patient over or near the heart thereof.

FIG. 20D depicts a patient-wearable device 2030 that comprises a first sensing module 2032 and a second sensing module 2034, each of which is attached to an adhesive layer 2036. Adhesive layer 2036 may be wrapped, for example, around an ankle of a patient, such that each of first sensing module 2032 and second sensing module 2034 are on opposing sides of a dorsalis pedis artery of the patient, or around a lower leg of a patient, such that each of first sensing module 2032 and second sensing module 2034 are on opposing sides of a posterior tibial artery of the patient. In an alternate embodiment, each of first sensing module 2032 and second sensing module 2034 may have its own adhesive layer to facilitate such attachment.

In certain embodiments, first sensing module 2032 may comprise an RF transmitter (or RF transceiver) and second sensing module 2034 may comprise an RF receiver (or RF transceiver). First sensing module 2032 may be attached to one side of an arm or leg of a patient and second sensing module 2034 may be attached to an opposite side of the arm or leg of the patient.

Once in position, first sensing module 2032 may transmit RF signals to second sensing module 2034 over a period of time. Variations in the RF signals that are received by second sensing module 2034 from first sensing module 2023 may be analyzed to determine pulsatile blood flow in the portion of the arm or the leg between first sensing module 2032 and second sensing module 2034.

In some embodiments, multiple patient-wearable devices may be used to monitor for and detect a vascular occlusion (e.g., a blood clot) in a patient. For example, multiple patient-wearable devices may be used to monitor for and detect a vascular occlusion in a patient who has recently undergone a vascular medical procedure such as but not limited to angioplasty and stenting, atherectomy, arteriovenous (AV) fistula, AV graft, thrombectomy, vascular bypass surgery, or open carotid or femoral endarterectomy.

Such monitoring/detection of vascular occlusions may be achieved for example, by attaching multiple patient-wearable devices (e.g., multiple instances of patient-wearable device 100 or any of the other patient-wearable devices described herein) to different locations on a body of a patient (e.g., after the patient has undergone a vascular medical procedure) and monitoring pulse strength indications periodically or continuously generated by the devices over time to detect a differential decrease in pulse strength at one of the body locations. For example, a patient-wearable device may be attached to one or both sides of the neck of the patient (over one or both carotid arteries), to each wrist of the patient, and to each ankle of the patient. A processing unit (e.g., separate from the patient-wearable devices but communicatively connected thereto, or integrated into one or more of the patient-wearable devices) may receive via wired or wireless communication links pulse strength indications or measurements from each of the patient-wearable devices. The pulse strength indications may be received or collected periodically (e.g., every few seconds) or continuously. If a pulse strength indication from a particular patient-wearable device is observed to drop by more than a predetermined threshold below a baseline, then an alert may be generated as this may indicate the presence of a vascular occlusion in the patient. The baseline may be established for example, based on previous readings obtained from the same patient-wearable device and/or the other patient-wearable devices. By way of example, if a pulse strength indication or measurement generated by a patient-wearable device attached to the right wrist of the patient shows a sudden drop below the baseline by more than a predetermined threshold, this may indicate that there is a blood clot in the right arm of the patient. In such a case, the processing unit may generate one or more alerts. The alerts may comprise, for example and without limitation, one or more audible alerts, one or more visible alerts, one or more haptic alerts, and/or one or more electronic communications such as a notification that is sent to a monitoring device of a caregiver.

In certain embodiments, once a potential vascular occlusion has been identified in a body part of the patient (e.g., in the neck or in a limb of the patient) using the above-described method, a patient-wearable device such as that described above in reference to FIG. 8 may be attached to the relevant body part of the patient to better identify the precise location of the occlusion, e.g., by placing one or more devices in the distribution of the artery.

In some embodiments, multiple patient-wearable devices may be used to measure blood flow in an extremity of a patient relative to a baseline. Such a measurement may be useful, for example, in detecting peripheral artery disease (PAD) in a limb of a patient. For example, a first patient-wearable device may be attached to an upper arm of the patient (e.g., near the brachial artery) and a second patient-wearable device may be attached to an ankle of the patient and both devices may be used to generate one or more pulse rate indications or measurements. In further accordance with this example, the pulse strength indication(s)/measurement(s) generated on the upper arm may provide a baseline and the pulse strength indication(s)/measurement(s) generated on the ankle may be compared to this baseline. An index for the relevant leg may be generated, for example, by dividing a pulse strength indication for the ankle by the pulse strength indication of the upper arm. A relatively low index number may indicate narrowing or blockage of the arteries in the legs. Although this example involves attachment of the patient-wearable devices to the upper arm and ankle, this is not intended to be limiting. Different body locations may be used to provide the baseline pulse strength measurement and different body locations may be used to generate an extremity pulse strength measurement to compare to the baseline.

In some embodiments, a model trained via machine learning may be used to determine a pulse condition of a patient based on sensor data obtained by one or more patient-wearable devices or sensing modules. For example, a machine learning classifier may be trained and then used to determine whether, based on currently-captured sensor data, a particular component of a domain signal captured by a sensor comprises part of a pulse or subpulse or instead comprises non-relevant data. The data that is used to train the machine learning model may be obtained or derived from previously-captured sensor data associated with a patient (e.g., previously captured pulse, subpulse or ECG data). Furthermore, data obtained from sensors located on one part of a patient's body may be used to train a model that is then used to detect a pulse or subpulse on another party of the patient's body.

### III. Example Implementations for Assessing Cardiovascular Function of a Patient

Referring again to system 600 of FIG. 6, sensor data 638 (e.g., inertial motion sensor data, acoustic sensor data, ECG sensor data) transmitted from one or more of patient-wearable devices 602, 604, 606, 608, 610, 612, 614, 616, 618, 620, 622, 624 or 626 may be processed by processing unit 634 of computing device 628 (and/or by one or more processing units of the patient-wearable devices) to obtain various insights into the vascular physiology of a patient in a manner that avoids reliance on bulky and/or unreliable sensors such as photoplethysmography (PPG) sensors. For example, processing unit 634 of computing device 628 may be configured to process sensor data 638 to measure the pulse waveform associated with blood traveling through an artery during its radial displacement after cardiac contraction and modified by vascular tone. From such measurements, valuable information may be extrapolated about the strength of cardiac contraction and vascular tone to help care for a patient. For example, processing unit 634 may leverage such measurements to assess various aspects of a patient's cardiovascular function such as, but not limited to: heart rate, cardiac rhythm, pulse strength, perfusion index (PI), pulse transit time (PTT), pulse arrival time (PAT), pulse wave velocity (PWV), upstroke velocity (dP/dt), pulsatility index, and subpulse. Furthermore, processing unit 634 may be configured to process sensor data 638 to autonomously differentiate between pulseless electrical activity (PEA) and pseudo-PEA, a process that conventionally requires manual input. Processing unit 634 may then cause such assessments to be presented to a user (e.g., a clinician) via display device 630 or other suitable output device so that such assessments may be used thereby to guide medical decision making.

In certain embodiments, processing unit 634 may be configured to generate one or more of these assessments by synchronizing cardiac signal data generated by a first patient-wearable device with pulse wave data generated by a second patient-wearable device utilizing an inertial motion sensor (e.g., accelerometer and/or gyroscope, or an IMU integrating the same) and/or an acoustic sensor. The first patient-wearable device may be, for example, a patient-wearable device located over or otherwise proximal to the heart, such as patient-wearable device 602, whereas the second patient-wearable device may be, for example, a patient-wearable device located away from the heart, such as over or otherwise proximal to a carotid artery (e.g., patient-wearable device 604 or 606), a brachial artery (e.g., patient-wearable device 608 or 610), a radial artery (e.g., patient-wearable device 612 or 614), a femoral artery (e.g., patient-wearable device 616 or 618), a posterior tibial artery (e.g., patient-wearable device 620 or 622), a dorsalis pedis artery (e.g., patient-wearable device 624 or 626), a popliteal artery, or any other portion of a patient's arterial system.

The first patient-wearable device may include one or more sensors that are operable to detect heart activity and thereby generate cardiac signal data. For example, the first patient-wearable device may include an ECG sensor that is operable to detect electrical signals generated by the heart and thereby generate cardiac signal data in the form of ECG sensor data. As another example, the first patient wearable-device may include an acoustic sensor, such as a phonocardiogram (PCG) sensor, that is operable to detect acoustic signals generated by the heart and thereby generate cardiac signal data in the form of acoustic waveform data, such as PCG sensor data. Still further, the first patient-wearable device may include an inertial motion sensor, such as a seismocardiography (SCG) sensor, that is operable to detect vibrations caused by heartbeats and thereby generate cardiac signal data in the form of inertial waveform data, such as SCG sensor data. The first patient-wearable device may also include and utilize any combination of the foregoing sensor types to generate cardiac signal data and thereby facilitate a multi-modal approach to assessing heart function.

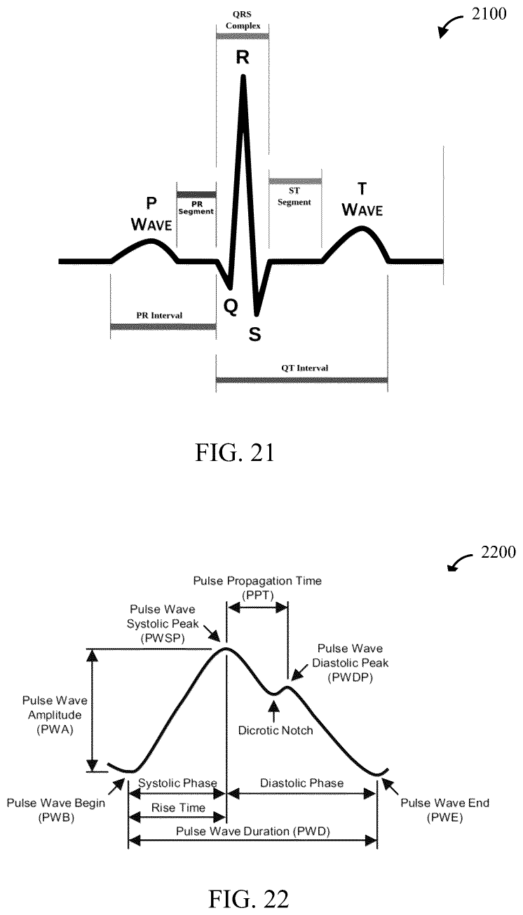

By way of example, FIG. 21 depicts an example cardiac waveform 2100 (in this case, an ECG waveform) that may be detected by the first patient-wearable device using an ECG sensor and FIG. 22 depicts an example pulse waveform 2200 that may subsequently be detected by the second patient-wearable device using an inertial motion sensor and/or acoustic sensor. In an embodiment, processing unit 634 may be configured to synchronize the cardiac signal data (in this case, ECG sensor data) generated by the first patient-wearable device with the pulse wave data generated by the second patient-wearable device by correlating the R wave of cardiac waveform 2100 with a corresponding upstroke, peak, and trough of pulse waveform 2200. However, this is only an example, and processing unit 634 may perform the synchronization based on correlating other features of cardiac waveform 2100 and pulse waveform 2200. Such synchronization may also be based on a time delay between cardiac waveform 2100 and pulse waveform 2200, or a time delay between features thereof. In embodiments, sensor data generated by each of the patient-wearable devices is time-stamped in a synchronized manner thereby enabling a time delay between a cardiac waveform feature and a corresponding pulse wave feature to be measured based on the timestamps respectively associated with such features.

In certain embodiments in which an inertial motion sensor is used to obtain the pulse wave data, the inertial motion sensor may be in direct contact with the skin of the patient or may be offset by a barrier that itself is in direct contact with the skin but that nevertheless enables the inertial motion sensor to measure radial displacement of an artery.

In certain embodiments, a patient-wearable device may utilize one or more acoustic sensors instead of or in addition to an inertial motion sensor to obtain pulse wave data. In a case in which both inertial motion and acoustic sensors are used, the acoustic sensor may provide built-in redundancy to enhance signal accuracy. Acoustic sensor(s) may also be used to provide specific information as it pertains to active, continuous monitoring of heart and lung sounds.

By utilizing inertial motion and/or acoustic sensors to detect pulse waves, embodiments described herein can collect robust physiological data in environments where conventional sensors (e.g., PPG) fail. Furthermore, embodiments may achieve additional reliability by synchronizing cardiac signal data obtained from a centrally-located (e.g., over or otherwise proximal to the heart) patient-wearable device with pulse wave data obtained from one or more peripherally-located (e.g., away from the heart, such as over or otherwise proximal to a carotid, brachial, radial, femoral, posterior tibial, dorsalis pedis, or popliteal artery) patient-wearable devices.

Although the foregoing describes the use of acoustic sensors to detect heart sounds and pulse wave vibrations, patient-wearable devices described herein may utilize acoustic sensors to also detect internal body sounds of all types, including but not limited to heart sounds, lung sounds, bowel sounds, and joint noise. Furthermore, embodiments described herein may be configured to process body sound information to automatically assess circulatory system function, respiratory system function, digestive system function, and/or musculoskeletal system function. Accordingly, embodiments described herein can automatically perform auscultation in support of diagnostics, monitoring a patient's status, and guiding medical procedures in a manner that avoids reliance on stethoscopes (which can be inaccurate due to ambient noise, lack of operator experience, poor operator hearing, and patient factors like obesity) and doppler ultrasound (the use of which may be impractical both due to expense and unwieldiness of devices). It should be noted that the patient-wearable device location may be determined based on the type of body sound being monitored (e.g., over the patient's heart for heart sounds, on the front, back and sides of the patient's chest for lung sounds, over the patient's abdomen for bowel sounds, or over a particular joint of the patient for joint noise). Additionally, any filtering used to improve the acoustic signal can be adjusted to better capture the specific body sound being monitored.

### Subpulse

As previously described, a subpulse is a weak pulse that cannot be reliably detected by manual palpation. When a subpulse is present in a patient, there can be variability in detection points between providers, as palpation relies on subjective tactile perception and can vary even for the same provider due to factors such as environment, stress, and patient or room temperature. This can have extremely negative consequences for patients.

In an embodiment, a system for detecting a subpulse of a patient may include a central patient-wearable device located over or otherwise proximal to the heart of the patient (e.g., on the chest) and one or more peripheral patient-wearable devices located away from the heart of the patient and over or otherwise proximal to a respective artery of the patient (e.g., over or otherwise proximal to a carotid artery, a brachial artery, a radial artery, a femoral artery, a posterior tibial artery, a dorsalis pedis artery, or a popliteal artery of the patient). For example, such a system may be system 600 of FIG. 6, the central patient-wearable device may be patient-wearable device 602, and the peripheral patient-wearable devices may comprise one or more of patient-wearable device 604 or 606 (proximal to a carotid artery), patient-wearable device 608 or 610 (proximal to a brachial artery), patient-wearable device 612 or 614 (proximal to a radial artery), patient-wearable device 616 or 618 (proximal to a femoral artery), patient-wearable device 620 or 622 (proximal to a posterior tibial artery), or patient-wearable device 624 or 626 (proximal to a dorsalis pedis artery). However, these are only examples and are not intended to be limiting.

In further accordance with this embodiment, the central patient-wearable device (e.g., patient-wearable device 602) includes one or more sensors that are operable to generate cardiac signal data (e.g., ECG sensor data, acoustic waveform (e.g., PCG sensor) data, and/or inertial waveform (e.g., SCG sensor) data), while each peripheral patient-wearable device (e.g., one or more of patient-wearable devices 604, 606, 608, 610, 612, 614, 616, 618, 620, 622, 624 or 626) includes an inertial motion sensor and/or acoustic sensor that are operable to generate pulse wave data. In embodiments in which an inertial motion sensor is used to obtain the pulse wave data, the inertial motion sensor may be in contact with soft tissue to detect movement of the relevant blood vessel in a direct path or perpendicular to the vessel. The inertial motion sensor may be in direct contact with the skin of the patient or may be offset by a barrier that itself is in direct contact with the skin but that nevertheless enables the inertial motion sensor to detect movement of the relevant blood vessel.

In still further accordance with this example, a processing unit is configured to receive the cardiac signal data from the central patient-wearable device and pulse wave data from at least one of the peripheral patient-wearable devices. The processing unit may be configured to utilize the cardiac signal data to filter the pulse wave data obtained from the peripheral patient-wearable device. For example, the processing unit may time-align R waves (or other features) of ECG waveforms present in the cardiac signal data with corresponding upstrokes, peaks, and troughs (or other features) of pulse waves present in the pulse wave data to identify time domain signal components that likely represent the patient's pulse and filter out time domain signal components that do not. In addition to using the cardiac signal data to perform time domain filtering of the pulse wave data, the processing unit may also be configured to obtain a frequency domain representation of the cardiac signal data and use such frequency domain representation to filter a frequency domain representation of the pulse wave data, thereby removing signal components that are likely to represent noise while preserving signal components that likely represent the patient's pulse. The processing unit may perform other types of filtering operations on the pulse wave data as deemed desirable.

As previously discussed herein, the processing of sensor data may be performed by a processing unit of a computing device (e.g., processing unit 634 of computing device 628) that is communicatively coupled to the various patient-wearable devices, and/or by a respective processing unit of one or more of the patient-wearable devices. For example, a single patient-wearable device (e.g., patient-wearable device 602) may generate the cardiac signal data while also collecting the pulse wave data from the peripheral patient-wearable device(s) and a processing unit thereof may operate to filter the pulse wave data using the cardiac signal data in the manner previously described. Alternatively, each peripheral patient-wearable device may generate its own pulse wave data while receiving the cardiac signal data from the same patient-wearable device (e.g., patient-wearable device 602) and a processing unit of each peripheral patient-wearable device may operate to filter its locally-captured pulse wave data using the cardiac signal data in the manner previously described. In either case, the filtered pulse wave data may be passed to a computing device (e.g., computing device 628) and used thereby to drive a presentation of patient pulse information to a practitioner (e.g., via display device 630 or other suitable output device).

The information presented by computing device 628 via display device 630 may comprise, for example, a binary indicator as to whether a pulse (including a subpulse) has been detected at each of the body locations of the peripheral patient-wearable devices, a graphical representation of pulse wave data over time as obtained from each of the peripheral patient-wearable devices, a pulse strength measurement detected at each of the body locations of the peripheral-wearable devices, a difference between pulse strength measurements detected at different body locations, or an indication that a pulse strength obtained at a particular body location or a difference between pulse strengths obtained at different body locations deviates from a norm or exceeds or falls below a threshold. A graphical representation of pulse wave data may be presented concurrently with a graphical representation of synchronized cardiac signal (e.g., ECG sensor) data over time. Visual indicators of pulse presence or non-presence may be presented in association with a visual indicator of the body location with which they are associated. For example, an image of a body of a patient may be annotated with a pulse or non-pulse indication at various body locations thereof. Such information may be updated in real-time or near real-time. Such information may be monitored by a user (e.g., a healthcare practitioner) to detect patient condition and trends and determine if medical treatment or intervention is required. In certain embodiments, such information may additionally or alternatively be displayed by one or more of the patient-wearable devices (e.g., a peripheral patient-wearable device may include an LED indicator that indicates whether a pulse has been detected at the corresponding body location or may include a more sophisticated (e.g., LCD, OLED, AMOLED) display that renders a graphical representation of pulse wave data obtained by the device over time).

Although the foregoing describes the detection of a subpulse in a manner that leverages cardiac signal data collected by a central patient-wearable device (e.g., patient-wearable device 602), it is noted that, as described elsewhere herein, a single patient-wearable device equipped with an inertial motion sensor and/or acoustic sensor may detect a pulse wave indicative of a subpulse without having access to such ECG sensor data.

### Perfusion Index (PI)

Perfusion index (PI) is a numerical value that reflects the ratio of pulsatile blood flow to non-pulsatile blood flow in an artery. PI provides an estimate of peripheral pulse strength, with higher values corresponding to better perfusion, and is helpful in assessing the perfusion of a patient in general or of a limb specifically. PI data can be used to formulate a differential diagnosis and treatment plan to help stabilize a patient, evaluate ongoing treatment efforts, or identify trends indicating potential for decompensation. Conventionally, PI is often measured by a pulse oximeter using photoplethysmography (PPG). PPG entails using a light source to illuminate the skin and a photosensor to measure the changes in light absorption or reflection that occur with each heartbeat. The principle behind PPG is that blood absorbs light more strongly than the surrounding tissue. However, PPG is inaccurate and unreliable in cases of poor contact, artificial or painted fingernails, dark skin color, dirty hands, light pollution, low blood pressure or low perfusion states, and other environmental factors. Poor PPG readings disrupt the PPG waveform, which provides inaccurate data for interpretation.

In certain conventional pulse oximeters, PI is calculated according to:

PI=(AC/DC)×100,

where AC is the amplitude of the pulsatile (arterial) component and DC is the non-pulsatile baseline (venous or tissue). A patient-wearable device as described herein may detect a pulse waveform at a given body location using an inertial motion sensor and/or acoustic sensor as discussed above (optionally filtered using cardiac signal data). A processing unit included in or connected to the patient-wearable device may calculate the PI using the above equation, wherein the processing unit obtains the pulsatile amplitude (AC) by measuring the pulse waveform (e.g., by measuring a peak-to-trough amplitude of the pulse waveform), and wherein the processing unit uses a baseline mechanical signal for DC (e.g., the average voltage level over one period of the pulse waveform).

This approach to obtaining the PI bypasses the need to rely on light (as PPG does), with its inherent shortcomings, and can be used to quantify the presence and quality of a pulse and trend changes in tissue perfusion, such as during CPR, shock states, resuscitation, trauma, and limb perfusion. The PI obtained in this manner can serve as a non-invasive guide to patient monitoring and resuscitation not currently available using conventional technology. Real-time, accurate and objective trending of this PI will allow providers to understand immediate impact of resuscitation treatments and alert providers to changes in a patient's clinical status, especially at the level of local tissue perfusion. For example, with continued reference to FIG. 6, if the PI provided by a patient-wearable device located over or otherwise proximal to a carotid artery (e.g., patient-wearable device 604 or 606) is high, representing good perfusion, but the PI concurrently provided by a patient-wearable device located over or otherwise proximal to a dorsalis pedis artery (e.g., patient-wearable device 624 or 626) and by a patient-wearable device located over or otherwise proximal to a posterior tibial artery (e.g., patient-wearable device 620 or 622) are low, that would represent poor tissue perfusion to the extremity. A clinician could then surmise that there is peripheral vasoconstriction and could assist in determining the type of shock the patient is experiencing or assess the reason for differences in extremity perfusion (right vs. left, arms vs. legs, or single location).

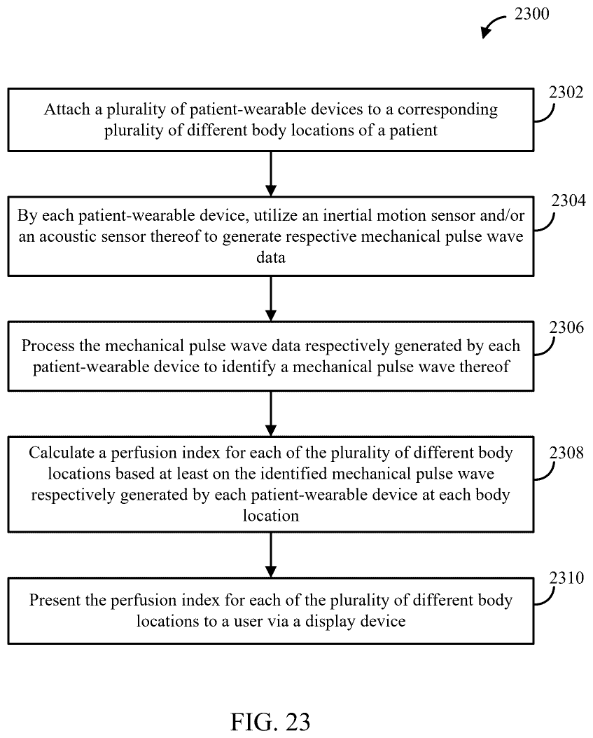

By way of further illustration, FIG. 23 depicts a flowchart 2300 of a method for concurrently measuring and monitoring a PI for each of a plurality of body locations of a patient, in accordance with an embodiment. The method of flowchart 2300 may be implemented, for example, by system 600 of FIG. 6, although this is only an example.

As shown in FIG. 23, the method of flowchart 2300 begins at step 2302, in which a plurality of patient-wearable devices are attached to a corresponding plurality of different body locations of a patient. This step may entail, for example, attaching one or more patient-wearable devices away from the heart of the patient such as over or otherwise proximal to a carotid artery (e.g., one or more of patient-wearable devices 604 or 606), and attaching one or more patient-wearable devices at body locations still further away from the heart of the patient such as over or otherwise proximal to a brachial artery, radial artery, femoral artery, dorsalis pedis artery, or posterior tibial artery (e.g., one or more of patient-wearable devices 612, 614, 616, 618, 620, 622, 624 or 626).

Step 2302 may also include attaching a central patient-wearable device that includes one or more cardiac signal sensors (e.g., an ECG sensor, an acoustic (e.g., PCG) sensor, and/or an inertial motion (e.g., SCG) sensor) over or otherwise proximal to the heart of the patient, such as on the chest of the patient (e.g., patient-wearable device 602).

At step 2304, each patient-wearable device in the plurality of patient-wearable devices utilizes an on-board inertial motion sensor (e.g., accelerometer and/or gyroscope, or an IMU integrating the same) and/or acoustic sensor to generate respective pulse wave data. In an embodiment, the pulse wave data respectively generated by each of the patient-wearable devices may be filtered using cardiac signal data generated by the one or more cardiac signal sensors of the central patient-wearable device in a manner that has been previously described. The pulse wave data may also be filtered using other filtering techniques as well.

At step 2306, the pulse wave data respectively generated by each patient-wearable device is processed to identify a pulse waveform thereof. For example, step 2306 may entail processing the pulse wave data generated by each patient-wearable device to identify a pulse waveform thereof that corresponds to the same arterial pulse pressure wave. In an embodiment, this may entail identifying in each set of pulse wave data a pulse waveform having a feature (e.g., an upstroke, peak and trough) that corresponds to a feature (e.g., R wave) of a cardiac signal waveform (e.g., an ECG waveform) present in cardiac signal data generated by the central patient-wearable device. This may be the R wave that immediately preceded the contraction of the heart that generated the pulse waveforms.

Alternatively, cardiac signal data may not be used to perform step 2306. For example, processing the pulse wave data respectively generated by each patient-wearable device to identify a pulse waveform thereof that corresponds to the same arterial pulse pressure wave may entail comparing a time delay between multiple different pulse waveforms obtained at different respective body locations and/or the shapes thereof to determine whether such pulse waveforms are associated with the same arterial pulse pressure wave.

At step 2308, a PI is calculated for each of the plurality of different body locations based at least on the identified pulse waveform respectively generated by each patient-wearable device at each body location. Thus, for example, a PI for a body location over or otherwise proximal to a carotid artery may be calculated based on the identified pulse waveform generated by patient-wearable device 604 or 606, a PI for a body location over or otherwise proximal to a dorsalis pedis artery may be calculated based on the identified pulse waveform generated by patient-wearable device 624 or 626, and a PI for a body location over or otherwise proximal to a posterior tibial artery may be calculated based on the identified pulse waveform generated by patient-wearable device 620 or 622. In each case, the PI may be calculated according to:

$$PI = ( AC / DC ) \times 100 ,$$

where AC is obtained by measuring a peak-to-trough amplitude of the relevant pulse waveform, and where DC is obtained by using a baseline mechanical signal (e.g., the average voltage level over one period of the pulse waveform).

At step 2310, the PI for each of the plurality of different body locations is presented to a user via a display device, or other output device. For example, the PI for each of the plurality of different body locations may be concurrently displayed on display device 630. The different PIs may be presented along with a visual indication of which body location they map to, so that PIs at different body locations may be compared. A computed difference between PIs at different body locations may be displayed, as well as an indication that a PI at a particular body location or a difference between PIs at different body locations deviates from a norm or exceeds or falls below a threshold. Each patient-wearable device at each body location may also display its local PI via an integrated display. The PIs may be updated on a real time or near real time basis. Trends in PIs over time at different body locations may also be presented so that a user may discern if PI is increasing or decreasing at a particular body location over time, e.g., to determine the quality of peripheral blood flow.

In the foregoing method of flowchart 2300, the processing of pulse wave data and the PI calculations may be carried out by a single processing unit, such as processing unit 634 of computing device 628, or by a processing unit of a single patient-wearable device (e.g., a processing unit of patient-wearable device 602). Alternatively, the processing of pulse wave data and the PI calculations may be carried out in a distributed fashion. For example, a processing unit of each of the plurality of patient-wearable devices may process its own locally-detected pulse waveforms and conduct its own local PI calculations. In such a scenario, if cardiac signal data is used for filtering and synchronization, it may be transmitted from a central patient-wearable device (e.g., patient-wearable device 602) to each of the plurality of patient-wearable devices for use thereby. Still other configurations are possible.

Although the foregoing describes the use of multiple patient-wearable devices to concurrently measure and monitor a PI for multiple body locations, it will be appreciated that in certain embodiments a single patient-wearable device may be used to measure and monitor a PI at a single body location. For example, a single-patient wearable device equipped with an inertial motion sensor and/or acoustic sensor may detect a pulse waveform at a body location, and a PI for that body location may be calculated based on the mechanical pulse waveform in a manner previously described and presented to a user.

### Pulse Arrival Time (PAT) and Pulse Transit Time (PTT)

The ability of embodiments to synchronize cardiac signal data with pulse wave data by, for example, aligning ECG R-waves with the corresponding upstroke, peak, and trough of pulse waves, can facilitate the calculation of pulse transit time (PTT), pulse arrival time (PAT) and pulse wave velocity (PWV). These are valuable measurements that give insight into a patient's state of perfusion globally or for a particular limb being monitored. PTT and PAT can act as non-invasive proxies for blood pressure, cardiac function, and vascular tone. PTT may be defined as the time taken for a pulse pressure wave to travel between two different points within the arterial system. PAT may be defined as the total time from the heart's electrical activation (often the R-wave of an ECG) to the arrival of the pulse wave at a peripheral site, such as a finger. PAT includes the PTT between the heart and the peripheral site plus the pre-ejection period (PEP), which is the time between the heart's electrical activation and the start of blood ejection into the aorta. Thus, it may be stated that

$$PAT = PTT + PEP .$$

PTT can serve as an estimate for blood pressure and vascular tone. These measurements are useful as a trend to help guide interventions that augment blood volume (e.g. fluid bolus) or vascular tone (e.g. vasopressors). A shorter PTT correlates with increased vascular tone, and PAT and PTT are inversely correlated with systolic blood pressure (SBP). As arteries dilate and vasomotor tone decreases, the pulse wave travels more slowly and the PTT increases. This data can then be used to guide acute resuscitative efforts with real-time validation (e.g. shortening of the PTT with the addition of vasopressors). In addition, a prolonged PAT suggests poor contractility or decreased preload. Interventions that impact this measurement, such as a fluid bolus, could be validated in real-time by noting a shortening of the PAT and validating the therapies rendered. Whereas conventional methods (e.g. PPG) for determining these measurements fail due to environmental factors, skin tone, etc., embodiments described herein allow for accurate monitoring in any setting by obtaining pulse wave data with an inertial motion sensor and/or acoustic sensor.

Embodiments described herein can measure PAT by, for example, determining the length of time from an ECG R wave detected at a first body location (e.g., over or otherwise proximal to the heart) by a first patient-wearable device (e.g., patient-wearable device 602) to an initial upstroke of a corresponding pulse wave detected at a second body location by a second patient-wearable device (e.g., any of patient-wearable devices 604, 606, 608, 610, 612, 614, 616, 68, 620, 622, 624 or 626).

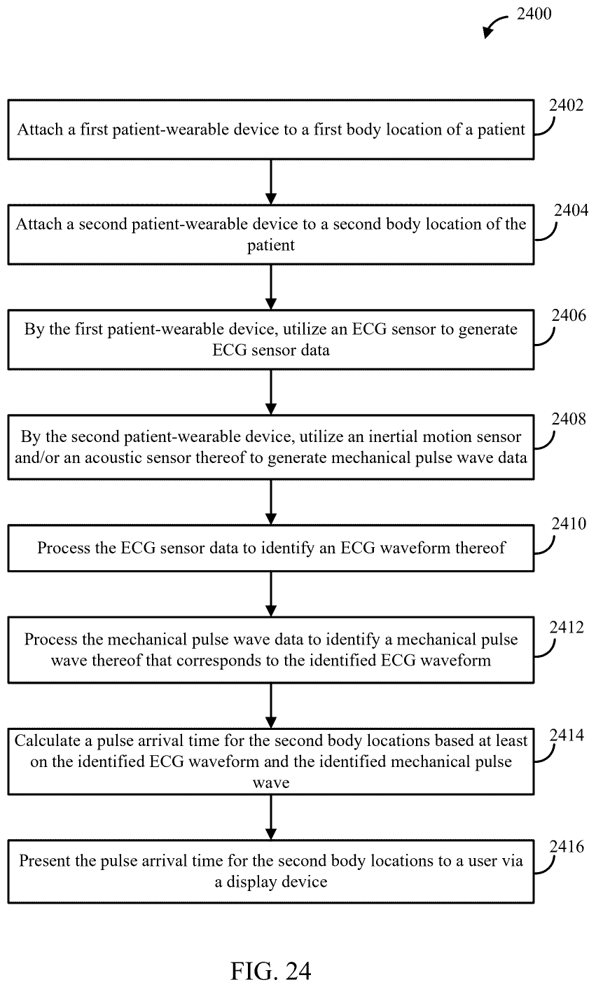

By way of illustration, FIG. 24 depicts a flowchart 2400 of a method for measuring and monitoring a PAT for a body location of a patient, in accordance with an embodiment. The method of flowchart 2400 may be implemented, for example, by system 600 of FIG. 6, although this is only an example.

As shown in FIG. 24, the method of flowchart 2400 begins at step 2402, in which a first patient-wearable device is attached to a first body location of a patient. The first body location may be, for example, over or otherwise proximal to the heart of the patient. In further accordance with this example and in reference to system 600 of FIG. 6, the first patient-wearable device may be patient-wearable device 602.

At step 2404, a second patient-wearable device is attached to a corresponding second body location of the patient. This step may entail, for example, attaching a patient-wearable device on a body location away from the heart of the patient such as over or otherwise proximal to a carotid artery (e.g., patient-wearable device 604 or 606), a radial artery (patient-wearable device 612 or 614), a femoral artery (e.g., patient-wearable device 616 or 618), a dorsalis pedis artery (patient-wearable device 624 or 626), or a posterior tibial artery (e.g., patient-wearable device 620 or 622).

At step 2406, the first patient-wearable device (e.g., patient-wearable device 602) utilizes one or more on-board cardiac signal sensors (e.g., an ECG sensor, an acoustic (e.g., PCG) sensor, and/or an inertial motion (e.g., SCG) sensor) to generate cardiac signal data.

At step 2408, the second patient-wearable utilizes an on-board inertial motion sensor (e.g., accelerometer and/or gyroscope, or an IMU integrating the same) and/or acoustic sensor to generate pulse wave data. In an embodiment, the pulse wave data generated by the second patient-wearable device may be filtered using cardiac signal data generated by the cardiac signal sensor(s) of the first patient-wearable device in a manner that has been previously described. The pulse wave data may also be filtered using other filtering techniques as well.

At step 2410, the cardiac signal data generated by the first patient-wearable device is processed to identify a cardiac waveform thereof. For example and without limitation, ECG sensor data generated by the first patient-wearable device may be processed to identify an ECG waveform thereof.

At step 2412, the pulse wave data generated by the second patient-wearable device is processed to identify a pulse waveform thereof that corresponds to the identified cardiac waveform. For example, this may entail identifying in the pulse wave data a pulse waveform having a feature (e.g., an upstroke, peak and trough) that corresponds to a feature of the identified cardiac waveform (e.g., an R wave of an ECG waveform).

At step 2414, a PAT is calculated for the second body location based at least on the identified cardiac waveform and the identified pulse wave. This step may entail, for example, calculating the PAT for the second body location as the time difference between a feature of the cardiac waveform (e.g., a peak of an R wave of an ECG waveform) and a corresponding feature (e.g., an initial upstroke or foot) of the identified pulse waveform. Calculating the time difference may entail, for example, determining an elapsed time between a timestamp associated with the relevant feature of the cardiac waveform and a timestamp associated with the relevant feature of the identified pulse waveform.

At step 2416, the PAT for the second body location is presented to a user via a display device, or other output device. For example, the PAT for the second body location may be displayed on display device 630. The PAT may be presented along with a visual indication of the second body location with which it is associated. The PAT may be updated on a real time or near real time basis. Trends in the PAT over time may also be presented so that a user may discern if the PAT is increasing or decreasing over time. An indication that the PAT deviates from a norm or exceed or falls below a threshold may also be displayed.

In embodiments, the method of flowchart 2400 may be extended to concurrently measure and monitor a PAT for each of multiple different body locations. For example, in addition to attaching the first patient-wearable device to the first body location of the patient (e.g., over or otherwise proximal to the heart of the patient), multiple second patient-wearable devices may be attached to multiple different second body locations and operate to generate pulse wave data at their respective body locations. Steps 2412 and 2414 may then be respectively performed for each of the second patient-wearable devices to determine a PAT for their respective body locations. Such PAT information may be presented to a user via a display device, or other output device. For example, the PAT for each of the second body locations may be concurrently displayed on display device 630, and each PAT may be presented along with a visual indication of the respective body location with which it is associated. A computed difference between PATs at different body locations may be displayed. An indication that a difference between PATs obtained at different body locations deviates from a norm or exceed or falls below a threshold may also be displayed. Each second patient-wearable device at each second body location may also display its local PAT via an integrated display. Each of the PATs may be updated on a real time or near real time basis. Trends in each PAT over time may also be presented so that a user may discern if each PAT is increasing or decreasing over time.

In embodiments, the processing of cardiac signal and pulse wave data and the PAT calculations may be carried out by a single processing unit, such as processing unit 634 of computing device 628, or by a processing unit of a single patient-wearable device. Alternatively, the processing of cardiac signal and pulse wave data and the PAT calculations may be carried out in a distributed fashion. For example, with respect to the embodiment described in the preceding paragraph involving multiple second patient-wearable devices at multiple respective body locations, a processing unit of each second patient-wearable device may process its own locally-detected pulse waveforms and conduct its own local PAT calculations. In such a scenario, cardiac signal data may be transmitted from the first patient-wearable device (e.g., patient-wearable device 602) to each of the second patient-wearable devices for use thereby in performing the calculation. Still other configurations are possible.

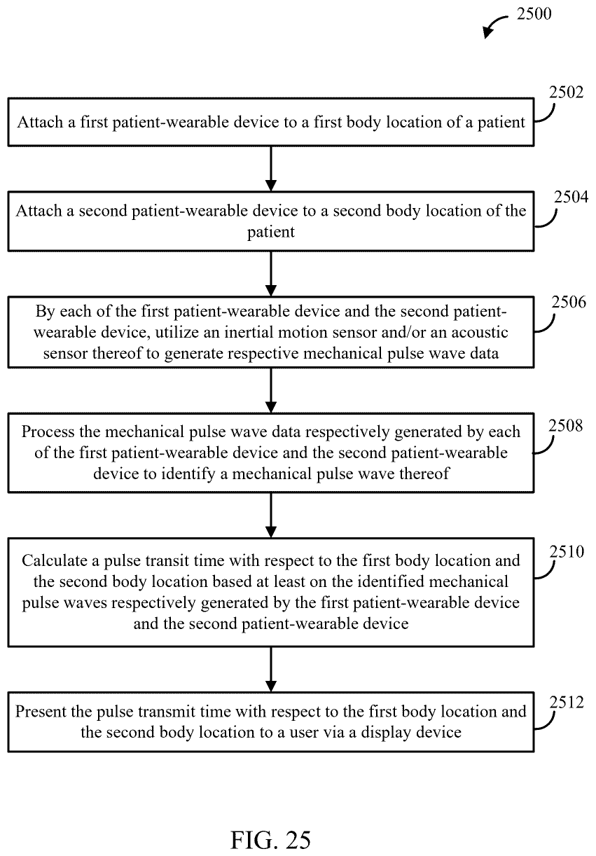

By way of further illustration, FIG. 25 depicts a flowchart 2500 of a method for measuring and monitoring a PTT with respect to two different body locations of a patient, in accordance with an embodiment. The method of flowchart 2500 may be implemented, for example, by system 600 of FIG. 6, although this is only an example.

As shown in FIG. 25, the method of flowchart 2500 begins at step 2502, in which a first patient-wearable device is attached to a first body location of a patient.

At step 2504, a second patient-wearable device is attached to a second body location of the patient.

The first and second body locations may be selected based on the type of PTT that is to be measured. For example, if the carotid-femoral PTT (cfPTT) is to be measured, the first patient-wearable device may be attached to a body location over or otherwise proximal to a carotid artery (e.g. patient-wearable device 604 or 606) and the second patient-wearable device may be attached to a body location over or otherwise proximal to a femoral artery (e.g., patient-wearable device 616 or 618). As another example, if the brachial-ankle PTT (baPTT) is to be measured, the first patient-wearable device may be attached to a body location over or otherwise proximal to a brachial artery (e.g. patient-wearable device 608 or 610) and the second patient-wearable device may be attached to a body location over or otherwise proximal to a posterial tibial artery (e.g., patient-wearable device 620 or 622) or a dorsalis pedis artery (e.g., patient-wearable device 624 or 626). As yet another example, if the brachial-radial PTT (brPTT) is to be measured, the first patient-wearable device may be attached to a body location over or otherwise proximal to a brachial artery (e.g. patient-wearable device 608 or 610) and the second patient-wearable device may be attached to a body location over or otherwise proximal to a radial artery (e.g., patient-wearable device 612 or 614). Other PTT measurements supported by other body location placements may include, for example and without limitation, femoral-popliteal, femoral-dorsalis pedis, femoral-posterior tibial, popliteal-dorsalis pedis, and popliteal-posterior tibial.

The method of flowchart 2500 may also include attaching a central patient-wearable device that includes one or more cardiac signal sensors (e.g., an ECG sensor, an acoustic (e.g., PCG) sensor, and/or an inertial motion (e.g., SCG) sensor) over or otherwise proximal to the heart of the patient, such as on the chest of the patient (e.g., patient-wearable device 602).

At step 2506, each of the first patient-wearable device and the second patient-wearable device utilizes an on-board inertial motion sensor (e.g., accelerometer and/or gyroscope, or an IMU integrating the same) and/or acoustic sensor to generate respective pulse wave data. In an embodiment, the pulse wave data respectively generated by each of the first patient-wearable device and the second patient-wearable device may be filtered using cardiac signal data generated by the one or more cardiac signal sensors of the central patient-wearable device in a manner that has been previously described. The pulse wave data may also be filtered using other filtering techniques as well.

At step 2508, the pulse wave data respectively generated by each of the first patient-wearable device and the second patient-wearable device is processed to identify a pulse waveform thereof. For example, step 2508 may entail processing the pulse wave data respectively generated by each of the first patient-wearable device and the second patient-wearable device to identify a pulse waveform thereof that corresponds to the same arterial pulse pressure wave. In an embodiment, this may entail identifying in each set of pulse wave data a pulse waveform having a feature (e.g., an upstroke, peak and trough) that corresponds to a feature (e.g., R wave) of a cardiac signal waveform (e.g., an ECG waveform) present in cardiac signal data generated by the central patient-wearable device. This may be the R wave that immediately preceded the contraction of the heart that generated the pulse waveforms.

Alternatively, cardiac signal data may not be used to perform step 2508. For example, processing the pulse wave data respectively generated by each of the first patient-wearable device and the second patient-wearable device to identify a pulse waveform thereof that corresponds to the same arterial pulse pressure wave may entail comparing a time delay between pulse waveforms obtained at the first and second body locations and/or the shapes thereof to determine whether such pulse waveforms are associated with the same arterial pulse pressure wave.

At step 2510, a PTT is calculated with respect to the first body location and the second body location based at least on the identified pulse waveforms respectively generated by first patient-wearable device and the second-patient wearable device. This step may entail, for example, calculating the PTT as the difference or delay between the time when a feature (e.g., foot or peak) appeared in the identified pulse waveform generated by the first patient-wearable device and the time when a corresponding feature (e.g., a corresponding foot or peak) appeared in the identified pulse waveform generated by the second patient-wearable device. Calculating the difference or delay may entail, for example, determining an elapsed time between a timestamp associated with the relevant feature in the identified pulse waveform generated by the first patient-wearable device and a timestamp associated with the corresponding relevant feature in the identified pulse waveform generated by the second patient-wearable device.

At step 2512, the PTT with respect to the first body location and the second body location is presented to a user via a display device, or other output device. For example, the PTT may be displayed on display device 630. The PTT may be presented along with a visual indication of the body locations used for its calculation. The PTT may also be presented by one or both of the first patient-wearable device or the second patient-wearable device via a respective integrated display. The PTT may be updated on a real time or near real time basis. Trends in the PTT over time may also be presented to the user. An indication that the PTT deviates from a norm or exceeds or falls below a threshold may also be displayed.

In the foregoing method of flowchart 2500, the processing of cardiac signal and pulse wave data and the PTT calculations may be carried out by a single processing unit, such as processing unit 634 of computing device 628, or by a processing unit of a single patient-wearable device. Alternatively, the processing of cardiac signal and pulse wave data and PTT calculation may be carried out in a distributed fashion across multiple processing units associated with multiple devices.

Rather than utilizing techniques described above with respect to flowchart 2500 to determine the PTT with respect to two different body locations, in an alternative embodiment, the PTT with respect to two different body locations may be determined based on the PAT associated with each of the two different body locations. For example, to determine cfPTT, techniques described above with respect to flowchart 2400 of FIG. 24 may be used to determine, for the same arterial pulse pressure wave, a PAT for a first body location that is over or otherwise proximal to a carotid artery and a PAT for a second body location that is over or otherwise proximal to a femoral artery. The cfPTT may then be estimated by subtracting the PAT for the first body location (the carotid PAT) from the PAT for the second body location (the femoral PAT).

In a further embodiment, a PTT between the heart and a particular body location may be determined by subtracting the PEP that precedes an arterial pulse pressure wave from the PAT for the particular body location with respect to the same arterial pulse wave (as determined in accordance with techniques of flowchart 2400). For example, in an embodiment, the PEP may be estimated as the time between the Q wave of an ECG waveform that immediately precedes the arterial pulse wave and the sound of the opening of the aortic valve. A patient-wearable device located over or otherwise proximal to the heart of the patient may include both an ECG sensor for detecting the relevant ECG waveform and an acoustic sensor (e.g., a PCG sensor) for detecting the sound of the opening of the aortic valve.

### Pulse Wave Velocity (PWV)

PWV is an important marker of cardiovascular health and arterial stiffness. PWV may be calculated by dividing the distance between two points in the arterial system by the time it takes the pulse wave to travel between the two arterial points (PTT), with the carotid-femoral locations most often cited in the medical literature. PWV is directly proportional to arterial stiffness, where an increased PWV indicates increased arterial stiffness or vasomotor tone and a decrease in PWV indicates decreased arterial stiffness or vasomotor tone. PWV can be used as a trend over time to determine progression of cardiovascular risk and changes in arterial function after interventions, such as medication implementation. Embodiments described herein can similarly monitor PWV non-invasively through the use of patient-wearable devices by measuring mechanical displacement of a blood vessel, providing clinicians valuable cardiovascular data to help improve the health of their patients.

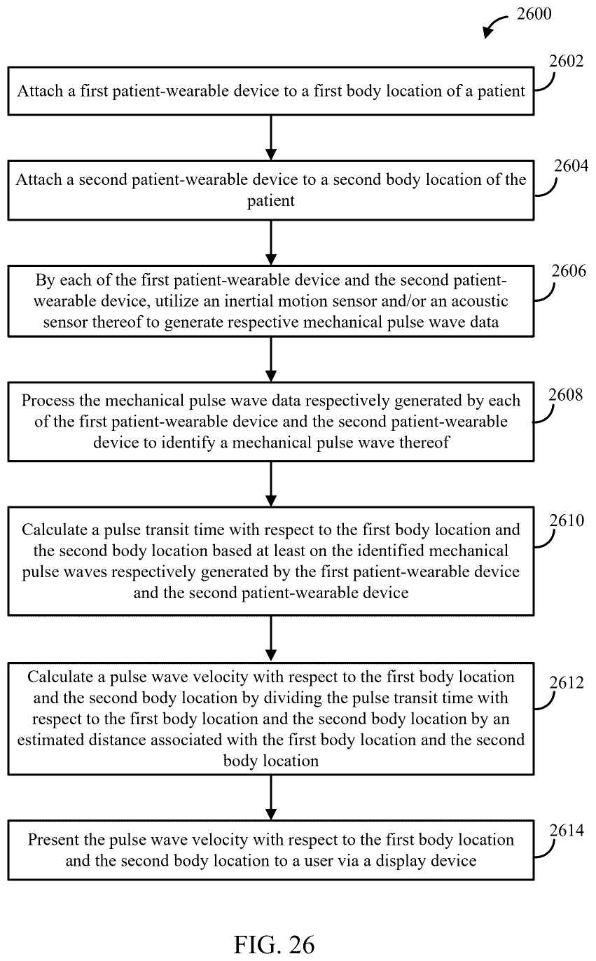

By way of illustration, FIG. 26 depicts a flowchart 2600 of a method for measuring and monitoring a PWV with respect to two different body locations of a patient, in accordance with an embodiment. The method of flowchart 2600 may be implemented, for example, by system 600 of FIG. 6, although this is only an example.

As shown in FIG. 26, the method of flowchart 2600 begins at step 2602, in which a first patient-wearable device is attached to a first body location of a patient.

At step 2604, a second patient-wearable device is attached to a second body location of the patient.

The first and second body locations may be selected based on the type of PWV that is to be measured. For example, if the carotid-femoral PWV (cfPWV) is to be measured, the first patient-wearable device may be attached to a body location over or otherwise proximal to a carotid artery (e.g. patient-wearable device 604 or 606) and the second patient-wearable device may be attached to a body location over or otherwise proximal to a femoral artery (e.g., patient-wearable device 616 or 618). As another example, if the brachial-ankle PWV (baPWV) is to be measured, the first patient-wearable device may be attached to a body location over or otherwise proximal to a brachial artery (e.g. patient-wearable device 608 or 610) and the second patient-wearable device may be attached to a body location over or otherwise proximal to a posterial tibial artery (e.g., patient-wearable device 620 or 622) or over or otherwise proximal to a dorsalis pedis artery (e.g., patient-wearable device 624 or 626). As yet another example, if the brachial-radial PWV is to be measured, the first patient-wearable device may be attached to a body location over or otherwise proximal to a brachial artery (e.g. patient-wearable device 608 or 610) and the second patient-wearable device may be attached to a body location over or otherwise proximal to a radial artery (e.g., patient-wearable device 612 or 614). Other PWV measurements supported by other body location placements may include, for example and without limitation, femoral-popliteal, femoral-dorsalis pedis, femoral-posterior tibial, popliteal-dorsalis pedis, and popliteal-posterior tibial.

The method of flowchart 2600 may also include attaching a central patient-wearable device that includes one or more cardiac signal sensors (e.g., an ECG sensor, an acoustic (e.g., PCG) sensor, and/or an inertial motion (e.g., SCG) sensor) over or otherwise proximal to the heart of the patient, such as on the chest of the patient (e.g., patient-wearable device 602).

At step 2606, each of the first patient-wearable device and the second patient-wearable device utilizes an on-board inertial motion sensor (e.g., accelerometer and/or gyroscope, or an IMU integrating the same) and/or acoustic sensor to generate respective pulse wave data. In an embodiment, the pulse wave data respectively generated by each of the first patient-wearable device and the second patient-wearable device may be filtered using cardiac signal data generated by the one or more cardiac signal sensors of the central patient-wearable device in a manner that has been previously described. The pulse wave data may also be filtered using other filtering techniques as well.

At step 2608, the pulse wave data respectively generated by each of the first patient-wearable device and the second patient-wearable device is processed to identify a pulse waveform thereof. For example, step 2608 may entail processing the pulse wave data respectively generated by each of the first patient-wearable device and the second patient-wearable device to identify a pulse waveform thereof that corresponds to the same arterial pulse pressure wave. In an embodiment, this may entail identifying in each set of pulse wave data a pulse waveform having a feature (e.g., an upstroke, peak and trough) that corresponds to a feature (e.g., R wave) of a cardiac signal waveform (e.g., an ECG waveform) present in cardiac signal data generated by the central patient-wearable device. This may be the R wave that immediately preceded the contraction of the heart that generated the pulse waveforms.

Alternatively, cardiac signal data may not be used to perform step 2608. For example, processing the pulse wave data respectively generated by each of the first patient-wearable device and the second patient-wearable device to identify a pulse waveform thereof that corresponds to the same arterial pulse pressure wave may entail comparing a time delay between pulse waveforms obtained at the first and second body locations and/or the shapes thereof to determine whether such pulse waveforms are associated with the same arterial pulse pressure wave.

At step 2610, a PTT is calculated with respect to the first body location and the second body location based at least on the identified pulse waveforms respectively generated by first patient-wearable device and the second-patient wearable device. This step may entail, for example, calculating the PTT as the difference or delay between the time when a feature (e.g., foot or peak) appeared in the identified pulse waveform generated by the first patient-wearable device and the time when a corresponding feature (e.g., a corresponding foot or peak) appeared in the identified pulse waveform generated by the second patient-wearable device. Calculating the difference or delay may entail, for example, determining an elapsed time between a timestamp associated with the relevant feature in the identified pulse waveform generated by the first patient-wearable device and a timestamp associated with the corresponding relevant feature in the identified pulse waveform generated by the second patient-wearable device.

At step 2612, a PWV is calculated with respect to the first body location and the second body location by dividing the PTT determined in step 2610 by an estimated distance associated with the first body location and the second body location. The estimated distance associated with the first body location and the second body location may be determined in a variety of ways as would be appreciated by persons skilled in the relevant art(s). The estimated distance associated with the first body location and the second body location may be an estimate of the physical or arterial distance between the first body location and the second body location. Alternatively, the estimated distance associated with the first body location and the second body location may be a difference between an estimate of the physical or arterial distance between the heart and the second body location and an estimate of the physical or arterial distance between the heart and the first body location. Still other approaches may be used to obtain the estimated distance. In embodiments in which the patient-wearable devices include RF (e.g., Bluetooth) transceivers, the estimated distance may be obtained by using methods such as Received Signal Strength Indicator (RSSI) or Channel Sounding (CS). If three or more patient-wearable devices with RF capabilities are present, then trilateration, triangulation or multilateration techniques may be used to improve distance calculations.

At step 2614, the PWV with respect to the first body location and the second body location is presented to a user via a display device, or other output device. For example, the PWV may be displayed on display device 630. The PWV may be presented along with a visual indication of the body locations used for its calculation. The PWV may also be presented by one or both of the first patient-wearable device or the second patient-wearable device via a respective integrated display. The PWV may be updated on a real time or near real time basis. Trends in the PWV over time may also be presented to the user. An indication that the PWV deviates from a norm or exceeds or falls below a threshold may also be displayed.

In the foregoing method of flowchart 2600, the processing of cardiac signal and pulse wave data and the PTT/PWV calculations may be carried out by a single processing unit, such as processing unit 634 of computing device 628, or by a processing unit of a single patient-wearable device. Alternatively, the processing of cardiac signal and pulse wave data and the PTT/PWV calculations may be carried out in a distributed fashion across multiple processing units associated with multiple devices.

### Upstroke Velocity

Upstroke velocity (dP/dt) is the rate at which pressure rises in an artery during ventricular ejection which is represented by the slope of the pulse waveform upstroke measured from the trough during diastole to the systolic peak of the pulse waveform. In embodiments, patient-wearable devices located at different body locations (e.g., different peripheral body locations) may be used to measure the upstroke velocity at each such body location. A high upstroke velocity represents strong contractility, while a low value represents poor cardiac contractility. Low values share an array of differential diagnoses for shock states (e.g., cardiogenic, hypovolemic, etc.). By enabling trends in upstroke velocity to be monitored over time, embodiments described herein can serve as an early warning system for patients in decline and also help guide resuscitative measures in real-time. Upstroke velocity can be a marker of worsening cardiac function which is valuable both in-hospital and out-of-hospital.

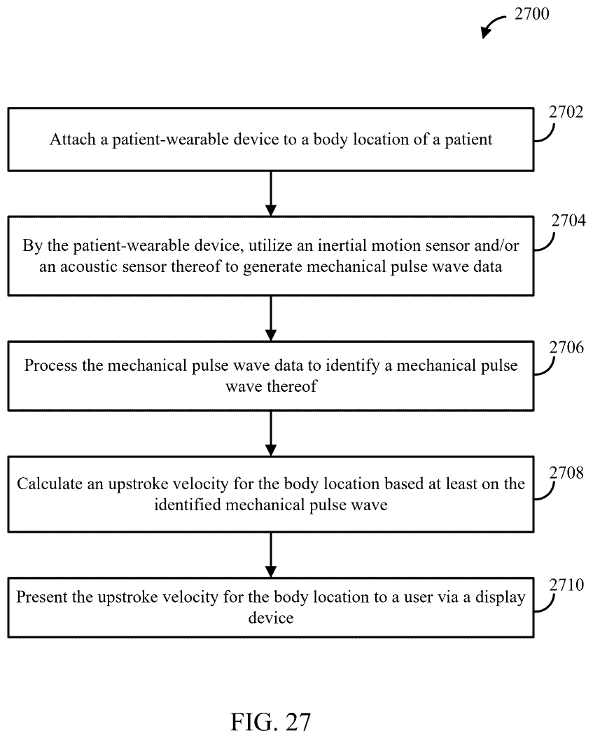

By way of illustration, FIG. 27 depicts a flowchart 2700 of a method for measuring and monitoring an upstroke velocity at a body location of a patient, in accordance with an embodiment. The method of flowchart 2700 may be implemented, for example, by system 600 of FIG. 6, although this is only an example.

As shown in FIG. 27, the method of flowchart 2700 begins at step 2702, in which a patient-wearable device is attached to a body location of a patient. The body location of the patient may comprise, for example, a body location over or otherwise proximal to the heart (e.g. patient-wearable device 602), a carotid artery (e.g. patient-wearable device 604 or 606), a brachial artery (e.g. patient-wearable device 608 or 610), a radial artery (e.g. patient-wearable device 612 or 614), a femoral artery (e.g. patient-wearable device 616 or 618), a posterior tibial artery (e.g. patient-wearable device 620 or 622), or a dorsalis pedis artery (e.g. patient-wearable device 624 or 626), although these are only examples.

At step 2704, the patient wearable device utilizes an on-board inertial motion sensor and/or acoustic sensor to generate pulse wave data. In an embodiment, the pulse wave data generated by the patient-wearable device may be filtered using cardiac signal data in a manner that has been previously described. The cardiac signal data may be generated, for example, by one or more cardiac signal sensors (e.g., ECG sensor, acoustic (e.g., PCG) sensor, and/or inertial motion (e.g., SCG) sensor) also included in the patient-wearable device or by one or more cardiac signal sensors of a different patient-wearable device that is concurrently attached to a different body location of the patient. The pulse wave data may also be filtered using other filtering techniques as well.

At step 2706, the pulse wave data is processed to identify a pulse waveform thereof.

At step 2708, an upstroke velocity is calculated for the body location based at least on the identified pulse waveform. For example, the upstroke velocity may be calculated by calculating a slope of the pulse waveform upstroke measured from the trough during diastole to a systolic peak of the mechanical pulse waveform (see, e.g., FIG. 22).

At step 2710, the upstroke velocity for the body location is presented to a user via a display device, or other output device. For example, the upstroke velocity for the body location may be displayed on display device 630. The upstroke velocity may be presented along with a visual indication of the body location with which it is associated. The upstroke velocity may also be presented by the patient-wearable device via an integrated display. The upstroke velocity may be updated on a real time or near real time basis. Trends in the upstroke velocity over time may also be presented so that a user may discern if the upstroke velocity is increasing or decreasing over time. An indication that the upstroke velocity deviates from a norm or exceeds or falls below a threshold may also be displayed.

In embodiments, the method of flowchart 2700 may be extended to concurrently measure and monitor an upstroke velocity for each of multiple different body locations. For example, multiple patient-wearable devices may be attached to multiple different body locations and operate to generate pulse wave data at their respective body locations. Steps 2706 and 2708 may then be respectively performed for each of the multiple patient-wearable devices to determine an upstroke velocity for their respective body locations. Each set of upstroke velocity measurements obtained at the different body locations may be synchronized to a same arterial pulse pressure wave, e.g., through synchronization of the underlying pulse waveforms to a cardiac signal waveform (e.g., an ECG waveform) and/or to each other. Such upstroke velocity information may be presented to a user via a display device, or other output device. For example, the upstroke velocity for each of the body locations may be concurrently displayed on display device 630, and each upstroke velocity may be presented along with a visual indication of the respective body location with which it is associated. A computed difference between upstroke velocities at different body locations may be displayed. An indication that a difference between upstroke velocities obtained at different body locations deviates from a norm or exceeds or falls below a threshold may also be displayed. The upstroke velocity at each body location may also be presented by the patient-wearable device at that body location via an integrated display. Each of the upstroke velocities may be updated on a real time or near real time basis. Trends in each upstroke velocity over time may also be presented so that a user may discern if each upstroke velocity is increasing or decreasing over time.

In the foregoing method of flowchart 2700, the processing of pulse wave data and the upstroke velocity calculations may be carried out by a single processing unit, such as processing unit 634 of computing device 628, or by a processing unit of a single patient-wearable device. Alternatively, the processing of pulse wave data and the upstroke velocity calculations may be carried out in a distributed fashion across multiple processing units associated with multiple devices.

### Pulsatility Index

Pulsatility index may be determined by the pulse waveform variability and captures data about the systolic upstroke, which is dependent on stroke volume or cardiac output, and the diastolic component, which is influenced by vascular resistance and compliance. Pulsatility index, therefore, captures the balance between cardiac ejection dynamics and peripheral vascular tone. A high pulsatility index can be seen with good cardiac contraction or elevated vasomotor tone (e.g., hypertension), while a low pulsatility index represents reduced cardiac contraction or low vasomotor tone (e.g., vasodilatory states such as sepsis). Changes over time are meaningful in critical care settings and can be used to determine shock type and guide resuscitation. Pulsatility index may be measured as follows:

Pulsatility Index=(peak systolic velocity−end diastolic velocity)/mean velocity.

Embodiments described herein may not directly measure blood velocity, so a scalar technique may instead be used to calculate pulsatility index based on a single pulse waveform obtained using an inertial motion sensor and/or acoustic sensor. When pulsatility index is measured in this manner, it reflects arterial wall/tissue pulsatility but not direct blood velocity. Such measurements may be influenced by local compliance, probe coupling and motion. However, as a trend, pulsatility index measured in this manner is highly useful as a proxy for cardiac output or vascular resistance. Using the above-referenced scalar technique for measurement, pulsatility index may be calculated as follows:

Pulsatility index=(peak systole−peak diastole/mean over cycle).

In embodiments, a single patient-wearable device attached to a given body location may be used to obtain pulsatility index data at the given body location to hemodynamically monitor a patient and help monitor for deterioration over time. A second patient-wearable device (e.g., attached over or proximal to the heart) may optionally be utilized to collect cardiac signal data (e.g., ECG sensor data, acoustic (e.g., PCG) sensor data, and/or inertial motion (e.g., SCG) sensor data) that can be used to help enhance data accuracy and provide other markers of hemodynamics (e.g. PAT, PTT, etc. as discussed above). Embodiments can therefore serve as a robust tool to not only monitor physiologic data such as cardiac signal and hemodynamics but be used more broadly to identify patients at risk or who are developing an acute issue. The data can then serve as a real-time marker for endpoints of resuscitation in any setting, either locally or remotely.

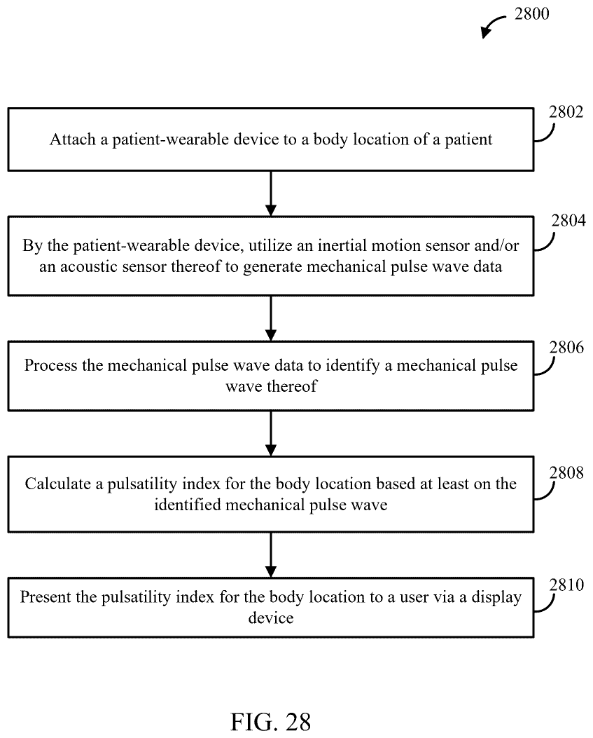

By way of illustration, FIG. 28 depicts a flowchart 2800 of a method for measuring and monitoring a pulsatility index at a body location of a patient, in accordance with an embodiment. The method of flowchart 2800 may be implemented, for example, by system 600 of FIG. 6, although this is only an example.

As shown in FIG. 28, the method of flowchart 2800 begins at step 2802, in which a patient-wearable device is attached to a body location of a patient. The body location of the patient may comprise, for example, a body location over or otherwise proximal to the heart (e.g. patient-wearable device 602), a carotid artery (e.g. patient-wearable device 604 or 606), a brachial artery (e.g. patient-wearable device 608 or 610), a radial artery (e.g. patient-wearable device 612 or 614), a femoral artery (e.g. patient-wearable device 616 or 618), a posterior tibial artery (e.g. patient-wearable device 620 or 622), or a dorsalis pedis artery (e.g. patient-wearable device 624 or 626), although these are only examples.

At step 2804, the patient wearable device utilizes an on-board inertial motion sensor and/or acoustic sensor to generate pulse wave data. In an embodiment, the pulse wave data generated by the patient-wearable device may be filtered using cardiac signal data in a manner that has been previously described. The cardiac signal data may be generated, for example, by one or more cardiac signal sensors (e.g., an ECG sensor, acoustic (e.g., PCG) sensor, and/or inertial motion (e.g., SCG) sensor) also included in the patient-wearable device or by one or more cardiac signal sensors of a different patient-wearable device that is concurrently attached to a different body location of the patient. The pulse wave data may also be filtered using other filtering techniques as well.

At step 2806, the pulse wave data is processed to identify a pulse waveform thereof.

At step 2808, a pulsatility index is calculated for the body location based at least on the identified pulse waveform. For example, the pulsatility index may be calculated as follows:

Pulsatility index=(peak systole−peak diastole/mean over cycle),

where peak systole is the amplitude of the systolic peak of the identified pulse waveform, peak diastole is the amplitude of the diastolic peak of the identified pulse waveform, and mean over cycle is the mean amplitude of the identified pulse waveform over a single cycle. In embodiments, cardiac signal data generated, for example, by cardiac signal sensor(s) also included in the patient-wearable device or by cardiac signal sensor(s) of a different patient-wearable device that is concurrently attached to a different body location of the patient, may be used to help differentiate between the systolic and diastolic phase of the pulse waveform. For example, the beginning of the systolic phase may correlate to a peak of an R wave of a synchronized ECG waveform (which signifies the onset of ventricular depolarization), whereas the beginning of the diastolic phase may correlate to an end of a T-wave of the synchronized ECG waveform (which signifies the completion of ventricular repolarization and the beginning of ventricular relaxation).

At step 2810, the pulsatility index for the body location is presented to a user via a display device, or other output device. For example, the pulsatility index for the body location may be displayed on display device 630. The pulsatility index may be presented along with a visual indication of the body location with which it is associated. The pulsatility index may also be presented by the patient-wearable device via an integrated display. The pulsatility index may be updated on a real time or near real time basis. Trends in the pulsatility index over time may also be presented so that a user may discern if the pulsatility index is increasing or decreasing over time. An indication that the pulsatility index deviates from a norm or exceeds or falls below a threshold may also be displayed.

In embodiments, the method of flowchart 2800 may be extended to concurrently measure and monitor a pulsatility index for each of multiple different body locations. For example, multiple patient-wearable devices may be attached to multiple different body locations and operate to generate pulse wave data at their respective body locations. Steps 2806 and 2808 may then be respectively performed for each of the multiple patient-wearable devices to determine a pulsatility index for their respective body locations. Each set of pulsatility index measurements obtained at the different body locations may be synchronized to a same arterial pulse pressure wave, e.g., through synchronization of the underlying pulse waveforms to a cardiac signal waveform (e.g., an ECG waveform) and/or to each other. Such pulsatility index information may be presented to a user via a display device, or other output device. For example, the pulsatility index for each of the body locations may be concurrently displayed on display device 630, and each pulsatility index may be presented along with a visual indication of the respective body location with which it is associated. A computed difference between pulsatility indexes at different body locations may be displayed. An indication that a difference between pulsatility indexes obtained at different body locations deviates from a norm or exceeds or falls below a threshold may also be displayed. The pulsatility index at each body location may also be presented by the patient-wearable device at that body location via an integrated display. Each of the pulsatility indexes may be updated on a real time or near real time basis. Trends in each pulsatility index over time may also be presented so that a user may discern if each pulsatility index is increasing or decreasing over time.

In the foregoing method of flowchart 2800, the processing of pulse wave data and the pulsatility index calculations may be carried out by a single processing unit, such as processing unit 634 of computing device 628, or by a processing unit of a single patient-wearable device. Alternatively, the processing of pulse wave data and the pulsatility index calculations may be carried out in a distributed fashion across multiple processing units associated with multiple devices.

### Pulseless Electrical Activity (PEA) and Pseudo-PEA

Pulseless electrical activity (PEA) and pseudo-PEA are critical states that must be differentiated rapidly and effectively. PEA is a state in which there is active cardiac electrical activity but no cardiac contraction and resultant pulse wave, leading to death. Pseudo-PEA occurs when cardiac electrical activity is present and the heart is contracting but it is contracting so weakly that the resultant pulse wave is not detectable by manual palpation (i.e., a subpulse). Because embodiments described herein can detect a subpulse, they may be used to differentiate between true PEA and pseudo-PEA, and thus may be relied on to specify the correct treatments for a patient in extremis or dying.

For example, a first patient-wearable device attached to a first body location and a second patient-wearable device attached to a second body location can work synchronously to provide a real-time or near real-time determination of whether a patient is exhibiting PEA or not, with the first patient-wearable device utilizing a cardiac signal sensor (e.g., an ECG sensor) to obtain cardiac signal data (e.g., ECG sensor data) indicative of cardiac electrical activity and the second patient-wearable device utilizing an inertial motion sensor and/or acoustic sensor to monitor the second body location for evidence of cardiac contractility in the form of a resultant mechanical pulse wave. The first patient-wearable device may be attached over or otherwise proximal to the heart of the patient, such as on the chest of the patient (e.g., patient-wearable device 602) whereas the second patient-wearable device may be placed in any number of suitable body locations, such as but not limited to, over or otherwise proximal to a carotid artery (e.g., patient-wearable device 604 or 606), a radial artery (e.g., patient-wearable device 612 or 614), or a femoral artery (e.g., patient-wearable device 616 or 618). These body locations are commonly used as the reference standard for pulse checks during cardiopulmonary resuscitation; however, other body locations may be used as well. Embodiments can be used in a continuous fashion not only to help determine pulse presence/absence but also to guide cardiopulmonary resuscitation (CPR) adequacy, as effective CPR provides arterial blood flow to the body. For example, if no pulse wave is detected near the carotid artery or the femoral artery during CPR, the CPR is not working and needs to be optimized to provide better perfusion, unless there is a separate reason for poor perfusion.

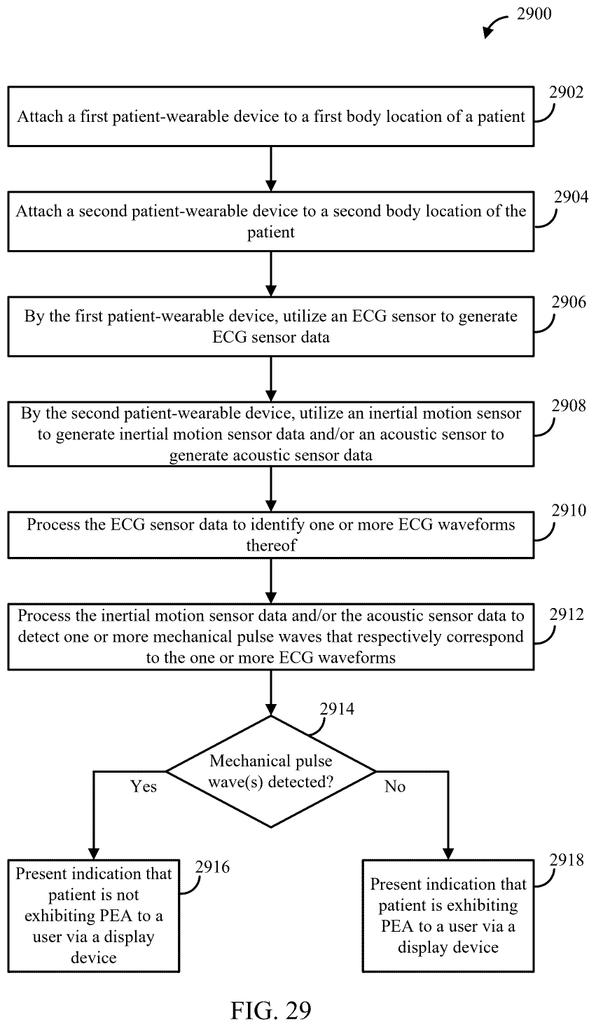

By way of illustration, FIG. 29 depicts a flowchart 2900 of a method for determining and indicating whether a patient is exhibiting PEA or is not exhibiting PEA (e.g., is exhibiting pseudo-PEA), in accordance with an embodiment. The method of flowchart 2900 may be implemented, for example, by system 600 of FIG. 6, although this is only an example.

As shown in FIG. 29, the method of flowchart 2900 begins at step 2902, in which a first patient-wearable device is attached to a first body location of a patient. The first body location may be, for example, over or otherwise proximal to the heart of the patient. In further accordance with this example and in reference to system 600 of FIG. 6, the first patient-wearable device may be patient-wearable device 602.

At step 2904, a second patient-wearable device is attached to a corresponding second body location of the patient. This step may entail, for example, attaching a patient-wearable device at a body location away from the heart of the patient such as over or otherwise proximal to a carotid artery (e.g., patient-wearable device 604 or 606), a radial artery (patient-wearable device 612 or 614), or a femoral artery (e.g., patient-wearable device 616 or 618), although these are only examples.

At step 2906, the first patient-wearable device (e.g., patient-wearable device 602) utilizes one or more on-board cardiac signal sensors (e.g., ECG sensor, acoustic (e.g., PCG) sensor, and/or inertial motion (e.g., SCG) sensor) to generate cardiac signal data. It will be appreciated that, while ECG sensor data directly detects the electrical activity of the heart, acoustic (e.g., PCG) sensor data and inertial motion (e.g., SCG) sensor data collected proximal to the heart can indirectly detect the electrical activity of the heart.

At step 2908, the second patient-wearable device utilizes an on-board inertial motion sensor (e.g., accelerometer and/or gyroscope, or an IMU integrating the same) and/or acoustic sensor to generate pulse wave data. In an embodiment, the pulse wave data generated by the second patient-wearable device may be filtered using the cardiac signal data generated by the first patient-wearable device. The pulse wave data may also be filtered using other filtering techniques as well.

At step 2910, the cardiac signal data generated by the first patient-wearable device is processed to identify one or more cardiac waveforms thereof. For example and without limitation, ECG sensor data generated by the first patient-wearable device may be processed to identify one or more ECG waveforms thereof.

At step 2912, the inertial motion sensor data and/or the acoustic sensor data is processed to determine whether the pulse wave data includes one or more pulse waveforms that respectively correspond to the identified one or more cardiac waveforms.

At decision step 2914, if it is at least determined that the pulse wave data includes the one or more pulse waveforms that respectively correspond to the one or more cardiac waveforms, then it is determined that the patient is not exhibiting PEA and an indication of that fact is presented to a user via a display device (e.g., display device 630) or other output device as shown at step 2916. In certain scenarios, the one or more pulse waveforms may represent a subpulse which has been detected by the second patient-wearable device. The indication that the patient is not exhibiting PEA may comprise, for example and without limitation: a visual (e.g., text or graphic) indicator that the patient is not exhibiting PEA, an indication that cardiac electrical activity is present at the first body location and that a pulse (e.g., a sub-pulse) is present at the second body location (e.g., concurrently displayed ECG waveforms and pulse waveforms), or any other indication suitable to convey that the patient is not exhibiting PEA.

However, if it is at least determined that the pulse wave data does not include the one or more pulse waveforms that respectively correspond to the identified one or more cardiac waveforms, then it is determined that the patient is exhibiting PEA and an indication of that fact is presented to a user via a display device (e.g., display device 630) or other output device as shown at step 2918. The indication that the patient is exhibiting PEA may comprise, for example and without limitation: a visual (e.g., text or graphic) indicator that the patient is exhibiting PEA, an indication that cardiac electrical activity is present at the first body location and that a pulse is not present at the second body location (e.g., concurrently displayed ECG waveforms and flat pulse signal), or any other indication suitable to convey that the patient is exhibiting PEA.

In embodiments, the method of flowchart 2400 may be extended to concurrently attempt to detect pulse waveforms at multiple different body locations. For example, multiple second patient-wearable devices may be attached to multiple different second body locations and operate to try and detect pulse waves that correspond to the cardiac waveforms being detected by the first patient-wearable device. In further accordance with such an embodiment, a decision that the patient is not exhibiting PEA may be declared whenever a pulse (including a subpulse) is detected by even a single one of the multiple second patient-wearable devices while a decision that patient is exhibiting PEA may be declared only when a pulse cannot be detected by all of the multiple second patient-wearable devices.

In embodiments, the processing of cardiac signal and pulse wave data and the PEA determinations may be carried out by a single processing unit, such as processing unit 634 of computing device 628, or by a processing unit of a single patient-wearable device. Alternatively, the processing of cardiac signal and pulse wave data and the PEA determinations may be carried out in a distributed fashion across multiple processing units associated with multiple devices.

In alternate embodiments, historical (e.g., previously-collected) cardiac signal information of the patient, and/or historical (e.g., previously-collected) cardiac signal information of one or more other patients or individuals, may be used rather than cardiac signal information collected in real time to perform one or both of (i) filtering of the pulse wave data collected by the second patient-wearable device using an inertial motion sensor and/or acoustic sensor and (ii) determining whether such pulse wave data includes pulse waveforms that correspond to patient cardiac waveforms and thus determining whether the patient is or is not exhibiting PEA. In accordance with such alternate embodiments, the first patient-wearable device need not be attached to the first body location of the patient (e.g., over or otherwise proximal to the heart of the patient) and used to capture cardiac signals at the time the PEA determination is to be made.

### Peripheral Vascular Monitoring (Occlusion)

Vascular arterial occlusion is a common and devastating pathology. Blood clots that form in the artery quickly lead to decreased blood flow and death of the tissue at that site and the sites distal to that occlusion. For example, a blood clot in the right femoral artery will quickly cause death of tissue in the right leg from the site of the femoral artery all the way to the toes. Delay in recognition or treatment can lead to amputation of extremities. Embodiments described herein can quickly and accurately detect arterial occlusion by sensing a decrease in pulse wave amplitude at a single body location or by detecting a relative decrease in pulse wave amplitude between two different body locations. As an example, after the insertion of an intra-aortic balloon pump there is risk of vascular occlusion due to the device's presence, dissection of the artery cannulated, or thrombosis due to stasis of blood. After insertion, therefore, it is necessary for healthcare providers to perform frequent checks of the pulse distal to the location of the device to ensure adequate perfusion. Manual palpation, however, is fallible, sometimes challenging, varies between healthcare providers, requires a manual task and resultant time away from other healthcare needs, and is frequently overlooked. Embodiments described herein may use patient-wearable devices to monitor peripheral pulses after insertion of any mechanical support device, like an intra-aortic balloon pump, thereby enabling healthcare professionals to offload this manual task and monitor the pulse in real-time, continuously, and trend data that would serve as an early warning system for developing poor perfusion or acute loss of perfusion to the affected limb.

As an alternative example, even when a device is not located within an artery, vascular disease can lead to a decline in perfusion to an extremity. Peripheral artery disease affects more than 236 million people in the United States and monitoring patients can be quite challenging. Embodiments described herein can utilize patient-wearable devices to serve as a comparator or perform individualized assessments of a patient's vascular health. Embodiments may be used to recognize declining markers of adequate perfusion and alert a patient's physician or provider to an impending problem, be used in the acute setting to monitor for worsening limb ischemia, or be used in postoperative states after fixing an occluded vessel to ensure patency of the vessel operated on and reopened. Postoperative vascular surgery care similarly requires frequent pulse checks, and using embodiments in this arena could save thousands of hours, if not more, annually of healthcare staff time in performing manual pulse checks and do so in a more sensitive and continuous fashion.

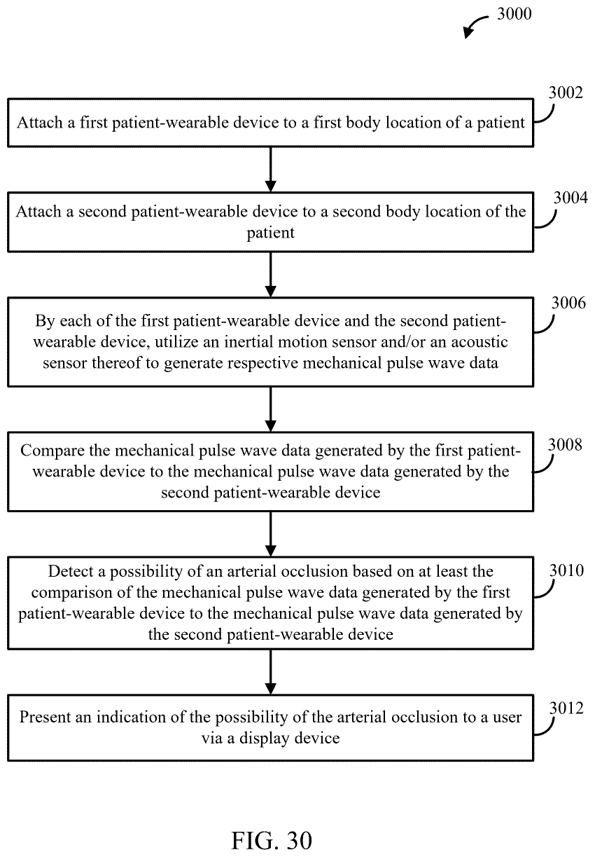

By way of illustration, FIG. 30 depicts a flowchart 3000 of method for detecting a possibility of an arterial occlusion in a patient and presenting an indication thereof, in accordance with an embodiment. The method of flowchart 3000 may be implemented, for example, by system 600 of FIG. 6, although this is only an example.

As shown in FIG. 30, the method of flowchart 3000 begins at step 3002, in which a first patient-wearable device is attached to a first body location of a patient.

At step 3004, a second patient-wearable device is attached to a corresponding second body location of the patient.

In an embodiment, the first body location of the patient and the second body location of the patient are both along with the path of a single artery, wherein the second body location is more distal from the heart of the patient than the first body location. Thus, for example, the first patient-wearable device may be placed over or otherwise proximal to the femoral artery and just below the inguinal canal, while the second patient-wearable device may be placed over or otherwise proximal to the femoral artery but at a more distal location with respect to the heart. In a further embodiment, the first body location of the patient may be over or otherwise proximal to an artery and the second body location of the patient may be over or otherwise proximal to a branch of that artery.

At step 3006, each of the first patient-wearable device and the second patient-wearable device utilize an on-board inertial motion sensor and/or acoustic sensor to generate respective pulse wave data. This step may entail detecting one or more pulse waveforms at the respective body location, or an absence thereof. The detection of pulse waveforms may be aided by using cardiac signal data provided by a concurrently-attached patient-wearable device in a manner previously described.

At step 3008, the pulse wave data generated by the first patient-wearable device is compared to the pulse wave data generated by the second patient-wearable device. This step may include, for example, synchronizing pulse waveforms detected by each device to the same arterial pulse pressure wave (e.g., by synchronizing the pulse waveforms with a cardiac waveform and/or each other) and comparing synchronized pulse waveforms with each other. This step may also include determining that the second patient-wearable device is unable to detect one or more pulse waveforms that correspond to one or more pulse waveforms detected by the first patient-wearable device.

At step 3010, based at least on the comparison of the pulse wave data generated by the first patient-wearable device to the pulse wave data generated by the second patient-wearable device, a possibility of an arterial occlusion is detected. For example, this step may entail detecting a possibility of an arterial occlusion if the amplitude of one or more pulse waveforms detected by the second patient-wearable device is lower than the amplitude of one or more synchronized pulse waveforms detected by the first patient-wearable device. In this case, the determination may take into account factors such as the magnitude of the difference (e.g., if the difference deviates from a norm or exceeds a threshold) and/or the time duration or number of pulse waveforms over which the difference continues. As another example, this step may entail detecting a possibility of an arterial occlusion if no pulse waveforms are being detected by the second patient-wearable device that correspond to pulse waveforms being detected by the first patient-wearable device. In this case, the determination may take into account factors such as the time duration or number of pulse waveforms detected by the first patient-wearable device for which the second patient-wearable device has detected no corresponding pulse waveforms.

In further embodiments, the detection of a possibility of an arterial occlusion may also take into account pulse wave data that is being generated by two patient-wearable devices that are respectively located proximal to arteries that are contralateral to each other. Thus, for example, the detection of a possibility of an arterial occlusion may take into account that strong pulse waveforms are being detected by a patient-wearable device that is proximal to the right femoral artery but weak or no pulse waveforms are being detected by a patient-wearable device that is proximal to the left femoral artery over the same time period. In embodiments, pulse wave data generated both upstream of an artery location as well as pulse wave data generated along a contralateral artery location may be used to detect the possibility of an arterial occlusion.

At step 3012, an indication of the possibility of the arterial occlusion is presented to a user via a display device (e.g., display device 630) or other output device. The indication of the possibility of the arterial occlusion may comprise for example: an indication of a patient-wearable device and/or the body location thereof at which weak or non-existent pulse waveforms have been detected, an indication of two patient-wearable devices and/or the body locations thereof for which a difference in pulse waveform strength has been detected, an indication of a limb, an artery, or arterial segment where the potential occlusion may exist, or the like. In embodiments, the indication of the possibility of the arterial occlusion may be presented by concurrently displaying pulse waveforms over time for the first patient-wearable device and second patient-wearable device whereby a difference in pulse strength between the two devices or the absence of a pulse at one of the devices is readily perceivable by a user. Still other methods for presenting an indication of the possibility of the arterial occlusion may be used.

In embodiments, the processing of pulse wave data and the detection of a possible arterial occlusion may be carried out by a single processing unit, such as processing unit 634 of computing device 628, or by a processing unit of a single patient-wearable device. Alternatively, the processing of pulse wave data and the detection of a possible arterial occlusion may be carried out in a distributed fashion across multiple processing units associated with multiple devices.

### Assessment of Cardiac Capture for External Transcutaneous Pacing (TCP)

Transcutaneous pacing (TCP) is a temporary, non-invasive medical procedure that uses external electrical impulses to regulate a patient's heart rate during emergencies like severe symptomatic bradycardia. In TCP, electrical pulses are delivered through an external pacemaker on the chest, stimulating the heart muscle to contract and maintain a sufficient cardiac output until a more stable, permanent solution can be implemented. The goal of TCP is the successful activation and forced contraction of the heart muscle by the external electrical impulse, which may be referred to as cardiac capture. However, failure to capture the electrical impulse of the heart is common with success dependent on factors such as patient size, chest wall impedance and electrode placement. Successful cardiac capture requires both electrical and mechanical capture, as electrical capture alone does not always guarantee that the heart is effectively pumping blood. In a 2024 article by Kimbrell et al. (“False Electrical Capture in Prehospital Transcutaneous Pacing by Paramedics: A Case Series”, Prehosp Emerg Care. 2024; 28(7):928-936), 82.6% of patients who underwent TCP had false electrical capture despite having documented mechanical capture by palpated pulse. This data suggests that providers are largely unsuccessful at TCP and that manual pulse checks can be misleading. Patients with false mechanical capture during TCP have higher rate of mortality. Therefore, it is imperative to accurately confirm mechanical perfusion.

Embodiments described herein may be utilized to assess mechanical capture, and thus confirm electrical capture, during TCP. For example, in an embodiment, one or more patient-wearable device may be placed over or otherwise proximal to an artery, such as but not limited to a carotid artery (e.g., patient-wearable device 604 or 606), a femoral artery (e.g., patient-wearable device 616 or 618), and/or a radial artery (e.g., patient-wearable device 612 or 614), prior to initiating TCP. Then after TCP is initiated, such patient-wearable devices may utilize an inertial motion sensor and/or an acoustic sensor to collect pulse wave data, and a processing unit (e.g., processing unit 634) may receive and analyze such pulse wave data to determine whether a pulse (including a sub-pulse) is present or absent at each of the respective body locations in a manner previously described. The processing unit may then cause such localized pulse determinations to be presented to the practitioner (e.g., via display device 630), who can adjust the electrical impulses of the TCP based on the presence or absence of a pulse. For example, the practitioner may gradually increase the milliamp (mA) output of the external pacemaker until both electrical capture (e.g., as determined based on the output of an ECG integrated into the external pacemaker) and mechanical capture (as indicated by the presence of a pulse at the body location(s)) are indicated. Since a high mA setting may cause patient discomfort, after both electrical capture and mechanical capture have been confirmed, the practitioner may slowly lower the mA to a more comfortable level that still maintains mechanical capture by observing the strength of the pulse wave at the respective body location(s) as the mA is reduced.

In scenarios in which the external pacemaker does not include an integrated ECG, a patient-wearable device that includes an ECG sensor may also be placed proximal to the heart of the patient to monitor for electrical capture during TCP, so long as it does not impede the placement of the external pacemaker. A system in accordance with such an embodiment (e.g., system 600) may thus be used to monitor for both electrical capture and mechanical capture during TCP.

### IV. Example Computer System Implementation

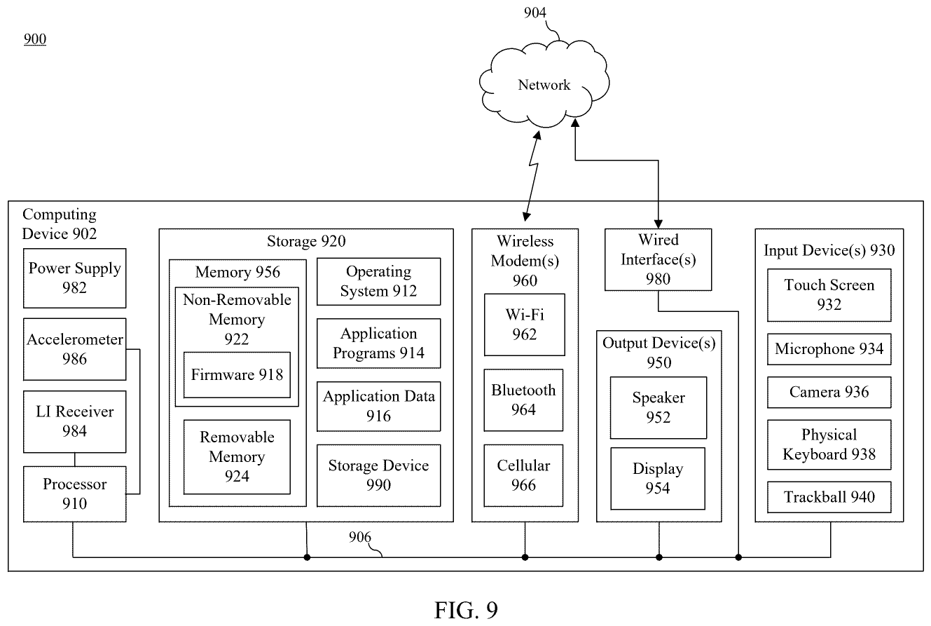

Examples of computing devices in which embodiments may be implemented are described as follows with respect to FIG. 9. FIG. 9 shows a block diagram of an exemplary computing environment 900 that includes a computing device 902. Computing device 902 is an example of computing device 628 of FIG. 6, which may include one or more of the components of computing device 902. In some embodiments, computing device 902 is communicatively coupled with devices (not shown in FIG. 9) external to computing environment 900 via network 904. Network 904 comprises one or more networks such as local area networks (LANs), wide area networks (WANs), enterprise networks, the Internet, etc., and may include one or more wired and/or wireless portions. Network 904 may additionally or alternatively include a cellular network for cellular communications. Computing device 902 is described in detail as follows

Computing device 902 can be any of a variety of types of computing devices. For example, computing device 902 may be a mobile computing device such as a handheld computer (e.g., a personal digital assistant (PDA)), a laptop computer, a tablet computer (such as an Apple iPad™), a hybrid device, a notebook computer (e.g., a Google Chromebook™ by Google LLC), a netbook, a mobile phone (e.g., a cell phone, a smart phone such as an Apple® iPhone® by Apple Inc., a phone implementing the Google® Android™ operating system, etc.), a wearable computing device (e.g., a head-mounted augmented reality and/or virtual reality device including smart glasses such as Google® Glass™, Oculus Rift® of Facebook Technologies, LLC, etc.), or other type of mobile computing device. Computing device 902 may alternatively be a stationary computing device such as a desktop computer, a personal computer (PC), a stationary server device, a minicomputer, a mainframe, a supercomputer, etc.

As shown in FIG. 9, computing device 902 includes a variety of hardware and software components, including a processor 910, a storage 920, one or more input devices 930, one or more output devices 950, one or more wireless modems 960, one or more wired interfaces 980, a power supply 982, a location information (LI) receiver 984, and an accelerometer 986. Storage 920 includes memory 956, which includes non-removable memory 922 and removable memory 924, and a storage device 990. Storage 920 also stores an operating system 912, application programs 914, and application data 916. Wireless modem(s) 960 include a Wi-Fi modem 962, a Bluetooth modem 964, and a cellular modem 966. Output device(s) 950 includes a speaker 952 and a display 954. Input device(s) 930 includes a touch screen 932, a microphone 934, a camera 936, a physical keyboard 938, and a trackball 940. Not all components of computing device 902 shown in FIG. 9 are present in all embodiments, additional components not shown may be present, and any combination of the components may be present in a particular embodiment. These components of computing device 902 are described as follows.

A single processor 910 (e.g., central processing unit (CPU), microcontroller, a microprocessor, signal processor, ASIC (application specific integrated circuit), and/or other physical hardware processor circuit) or multiple processors 910 may be present in computing device 902 for performing such tasks as program execution, signal coding, data processing, input/output processing, power control, and/or other functions. Processor 910 may be a single-core or multi-core processor, and each processor core may be single-threaded or multithreaded (to provide multiple threads of execution concurrently). Processor 910 is configured to execute program code stored in a computer readable medium, such as program code of operating system 912 and application programs 914 stored in storage 920. Operating system 912 controls the allocation and usage of the components of computing device 902 and provides support for one or more application programs 914 (also referred to as “applications” or “apps”). Application programs 914 may include common computing applications (e.g., e-mail applications, calendars, contact managers, web browsers, messaging applications), further computing applications (e.g., word processing applications, mapping applications, media player applications, productivity suite applications), one or more machine learning (ML) models, as well as applications related to the embodiments disclosed elsewhere herein.

Any component in computing device 902 can communicate with any other component according to function, although not all connections are shown for case of illustration. For instance, as shown in FIG. 9, bus 906 is a multiple signal line communication medium (e.g., conductive traces in silicon, metal traces along a motherboard, wires, etc.) that may be present to communicatively couple processor 910 to various other components of computing device 902, although in other embodiments, an alternative bus, further buses, and/or one or more individual signal lines may be present to communicatively couple components. Bus 906 represents one or more of any of several types of bus structures, including a memory bus or memory controller, a peripheral bus, an accelerated graphics port, and a processor or local bus using any of a variety of bus architectures.

Storage 920 is physical storage that includes one or both of memory 956 and storage device 990, which store operating system 912, application programs 914, and application data 916 according to any distribution. Non-removable memory 922 includes one or more of RAM (random access memory), ROM (read only memory), flash memory, a solid-state drive (SSD), a hard disk drive (e.g., a disk drive for reading from and writing to a hard disk), and/or other physical memory device type. Non-removable memory 922 may include main memory and may be separate from or fabricated in a same integrated circuit as processor 910. As shown in FIG. 9, non-removable memory 922 stores firmware 918, which may be present to provide low-level control of hardware. Examples of firmware 918 include BIOS (Basic Input/Output System, such as on personal computers) and boot firmware (e.g., on smart phones). Removable memory 924 may be inserted into a receptacle of or otherwise coupled to computing device 902 and can be removed by a user from computing device 902. Removable memory 924 can include any suitable removable memory device type, including an SD (Secure Digital) card, a Subscriber Identity Module (SIM) card, which is well known in GSM (Global System for Mobile Communications) communication systems, and/or other removable physical memory device type. One or more of storage device 990 may be present that are internal and/or external to a housing of computing device 902 and may or may not be removable. Examples of storage device 990 include a hard disk drive, a SSD, a thumb drive (e.g., a USB (Universal Serial Bus) flash drive), or other physical storage device.

One or more programs may be stored in storage 920. Such programs include operating system 912, one or more application programs 914, and other program modules and program data. Examples of such application programs may include, for example, computer program logic (e.g., computer program code/instructions) for implementing any of the functions ascribed herein to computing device 628, as well as any of steps 506, 508 or 510 of flowchart 500 as previously described herein.

Storage 920 also stores data used and/or generated by operating system 912 and application programs 914 as application data 916. Examples of application data 916 include web pages, text, images, tables, sound files, video data, and other data, which may also be sent to and/or received from one or more network servers or other devices via one or more wired or wireless networks. Storage 920 can be used to store further data including a subscriber identifier, such as an International Mobile Subscriber Identity (IMSI), and an equipment identifier, such as an International Mobile Equipment Identifier (IMEI). Such identifiers can be transmitted to a network server to identify users and equipment.

A user may enter commands and information into computing device 902 through one or more input devices 930 and may receive information from computing device 902 through one or more output devices 950. Input device(s) 930 may include one or more of touch screen 932, microphone 934, camera 936, physical keyboard 938 and/or trackball 940 and output device(s) 950 may include one or more of speaker 952 and display 954. Each of input device(s) 930 and output device(s) 950 may be integral to computing device 902 (e.g., built into a housing of computing device 902) or external to computing device 902 (e.g., communicatively coupled wired or wirelessly to computing device 902 via wired interface(s) 980 and/or wireless modem(s) 960). Further input devices 930 (not shown) can include a Natural User Interface (NUI), a pointing device (computer mouse), a joystick, a video game controller, a scanner, a touch pad, a stylus pen, a voice recognition system to receive voice input, a gesture recognition system to receive gesture input, or the like. Other possible output devices (not shown) can include piezoelectric or other haptic output devices. Some devices can serve more than one input/output function. For instance, display 954 may display information, as well as operating as touch screen 932 by receiving user commands and/or other information (e.g., by touch, finger gestures, virtual keyboard, etc.) as a user interface. Any number of each type of input device(s) 930 and output device(s) 950 may be present, including multiple microphones 934, multiple cameras 936, multiple speakers 952, and/or multiple displays 954.

One or more wireless modems 960 can be coupled to antenna(s) (not shown) of computing device 902 and can support two-way communications between processor 910 and devices external to computing device 902 through network 904, as would be understood to persons skilled in the relevant art(s). Wireless modem 960 is shown generically and can include a cellular modem 966 for communicating with one or more cellular networks, such as a GSM network for data and voice communications within a single cellular network, between cellular networks, or between the mobile device and a public switched telephone network (PSTN). Wireless modem 960 may also or alternatively include other radio-based modem types, such as a Bluetooth modem 964 (also referred to as a “Bluetooth device”) and/or Wi-Fi 962 modem (also referred to as an “wireless adaptor”). Wi-Fi modem 962 is configured to communicate with an access point or other remote Wi-Fi-capable device according to one or more of the wireless network protocols based on the IEEE (Institute of Electrical and Electronics Engineers) 802.11 family of standards, commonly used for local area networking of devices and Internet access. Bluetooth modem 964 is configured to communicate with another Bluetooth-capable device according to the Bluetooth short-range wireless technology standard(s) such as IEEE 802.15.1 and/or managed by the Bluetooth Special Interest Group (SIG).

Computing device 902 can further include power supply 982, LI receiver 984, accelerometer 986, and/or one or more wired interfaces 980. Example wired interfaces 980 include a USB port, IEEE 1394 (FireWire) port, a RS-232 port, an HDMI (High-Definition Multimedia Interface) port (e.g., for connection to an external display), a DisplayPort port (e.g., for connection to an external display), an audio port, an Ethernet port, and/or an Apple® Lightning® port, the purposes and functions of each of which are well known to persons skilled in the relevant art(s). Wired interface(s) 980 of computing device 902 provide for wired connections between computing device 902 and network 904, or between computing device 902 and one or more devices/peripherals when such devices/peripherals are external to computing device 902 (e.g., a pointing device, display 954, speaker 952, camera 936, physical keyboard 938, etc.). Power supply 982 is configured to supply power to each of the components of computing device 902 and may receive power from a battery internal to computing device 902, and/or from a power cord plugged into a power port of computing device 902 (e.g., a USB port, an A/C power port). LI receiver 984 may be used for location determination of computing device 902 and may include a satellite navigation receiver such as a Global Positioning System (GPS) receiver or may include other type of location determiner configured to determine location of computing device 902 based on received information (e.g., using cell tower triangulation, etc.). Accelerometer 986 may be present to determine an orientation of computing device 902.

Note that the illustrated components of computing device 902 are not required or all-inclusive, and fewer or greater numbers of components may be present as would be recognized by one skilled in the art. For example, computing device 902 may also include one or more of a gyroscope, barometer, proximity sensor, ambient light sensor, digital compass, etc. Processor 910 and memory 956 may be co-located in a same semiconductor device package, such as being included together in an integrated circuit chip, FPGA, or system-on-chip (SOC), optionally along with further components of computing device 902.

In embodiments, computing device 902 is configured to implement any of the above-described features of flowcharts herein. Computer program logic for performing any of the operations, steps, and/or functions described herein may be stored in storage 920 and executed by processor 910.

As used herein, the terms “computer program medium,” “computer-readable medium,” and “computer-readable storage medium,” etc., are used to refer to physical hardware media. Examples of such physical hardware media include any hard disk, optical disk, SSD, other physical hardware media such as RAMs, ROMs, flash memory, digital video disks, zip disks, MEMs (microelectronic machine) memory, nanotechnology-based storage devices, and further types of physical/tangible hardware storage media of storage 920. Such computer-readable media and/or storage media are distinguished from and non-overlapping with communication media and propagating signals (do not include communication media and propagating signals). Communication media embodies computer-readable instructions, data structures, program modules or other data in a modulated data signal such as a carrier wave. The term “modulated data signal” means a signal that has one or more of its characteristics set or changed in such a manner as to encode information in the signal. By way of example, and not limitation, communication media includes wireless media such as acoustic, RF, infrared and other wireless media, as well as wired media. Embodiments are also directed to such communication media that are separate and non-overlapping with embodiments directed to computer-readable storage media.

As noted above, computer programs and modules (including application programs 914) may be stored in storage 920. Such computer programs may also be received via wired interface(s) 980 and/or wireless modem(s) 960 over network 904. Such computer programs, when executed or loaded by an application, enable computing device 902 to implement features of embodiments discussed herein. Accordingly, such computer programs represent controllers of the computing device 902.

Embodiments are also directed to computer program products comprising computer code or instructions stored on any computer-readable medium or computer-readable storage medium. Such computer program products include the physical storage of storage 920 as well as further physical storage types.

### V. Conclusion

Various embodiments of a patient-wearable device for detecting a pulse condition of a patient and related systems, methods and computer program products have been described herein. As noted above, detecting a pulse condition of a patient may comprise detecting the presence or absence of a pulse of the patient, or the presence or absence of a subpulse of the patient. A subpulse should be understood to mean a spectrum of pulse that is less than reliability manually palpable. In an embodiment, detecting a subpulse may comprise detecting a pulse at a systolic blood pressure (SBP) of less than 80 mmHg. In a further embodiment, detecting a subpulse may comprise detecting a pulse at an SBP of less than 60 mmHg. In a still further embodiment, detecting a subpulse may comprise detecting a pulse at an SBP of less than 52 mmHg.

While various embodiments of the present disclosure have been described above, it should be understood that they have been presented by way of example only, and not limitation. It will be understood by those skilled in the relevant art(s) that various changes in form and details may be made therein without departing from the spirit and scope of the invention as defined in the appended claims. Accordingly, the breadth and scope of the present invention should not be limited by any of the above-described exemplary embodiments, but should be defined only in accordance with the following claims and their equivalents.

What is claimed is:

1. A system for determining and indicating whether a patient is exhibiting pulseless electrical activity (PEA) or is not exhibiting PEA, comprising:
a first patient-wearable device adapted for attachment to a first body location of the patient and configured to utilize one or more cardiac signal sensors thereof to generate cardiac signal data;
a second patient-wearable device that is adapted for attachment to a second body location of the patient and configured to utilize one or more of an inertial motion sensor or an acoustic sensor thereof to generate pulse wave data; and
a processing unit that is configured to:
receive the cardiac signal data from the first patient-wearable device and the pulse wave data from the second patient-wearable device;
process the cardiac signal data to identify one or more cardiac waveforms thereof;
process the pulse wave data to determine whether the pulse wave data includes one or more pulse waveforms that respectively correspond to the identified one or more cardiac waveforms;
generate a user-perceptible indication that the patient is not exhibiting PEA in response to at least determining that the pulse wave data includes one or more pulse waveforms that respectively correspond to the identified one or more cardiac waveforms; and
generate a user-perceptible indication that the patient is exhibiting PEA in response to at least determining that the pulse wave data does not include one or more pulse waveforms that respectively correspond to the identified one or more cardiac waveforms.

2. The system of claim 1, wherein the first body location of the patient is proximal to a heart of the patient and the second body location is away from the heart of the patient and proximal to one of a carotid artery of the patient, a radial artery of the patient, or a femoral artery of the patient.

3. The system of claim 1, wherein the cardiac signal sensor comprises one or more of an electrocardiogram (ECG) sensor, an acoustic sensor or an inertial motion sensor.

4. The system of claim 1, wherein the processing unit is further configured to filter the pulse wave data based on the cardiac signal data.

5. The system of claim 4, wherein the processing unit is configured to filter the pulse wave data based on the cardiac signal data by synchronizing the pulse wave data and the cardiac signal data.

6. The system of claim 1, wherein the processing unit is configured to generate the user-perceptible indication that the patient is not exhibiting PEA by rendering a visual indication that the patient is not exhibiting PEA to a display and wherein the processing unit is configured to generate the user-perceptible indication that the patient is exhibiting PEA by rendering a visual indication that the patient is exhibiting PEA to a display.

7. The system of claim 1, further comprising:
a third patient-wearable device that is adapted for attachment to a third body location of the patient and configured to utilize one or more of an inertial motion sensor or an acoustic sensor thereof to generate additional pulse wave data;
wherein the processing unit is further configured to:
receive the additional pulse wave data from the third patient-wearable device; and
process the additional pulse wave data to determine whether the additional pulse wave data includes one or more pulse waveforms that respectively correspond to the identified one or more cardiac waveforms;
wherein the processing unit is configured to generate the user-perceptible indication that the patient is not exhibiting PEA in response to at least determining that either one of the pulse wave data or the additional pulse wave data includes one or more pulse waveforms that respectively correspond to the identified one or more cardiac waveforms; and
wherein the processing unit is configured to generate the user-perceptible indication that the patient is exhibiting PEA in response to at least determining that both the pulse wave data and the additional pulse wave data do not include one or more pulse waveforms that respectively correspond to the identified one or more cardiac waveforms.

8. A method for determining and indicating that a patient is not exhibiting pseudo pulseless electrical activity (pseudo-PEA), comprising:
receiving cardiac signal data generated by one or more cardiac signal sensors of a first patient-wearable device that is attached to a first body location of the patient;
receiving pulse wave data generated by one or more of an inertial motion sensor or an acoustic sensor of a second patient-wearable device that is attached to a second body location of the patient;
processing the cardiac signal data to identify one or more cardiac waveforms thereof;
processing the pulse wave data to determine that the pulse wave data includes one or more pulse waveforms that respectively correspond to the identified one or more cardiac waveforms; and
generating a user-perceptible indication that the patient is not exhibiting PEA in response to at least determining that the pulse wave data includes one or more pulse waveforms that respectively correspond to the identified one or more cardiac waveforms.

9. The method of claim 8, wherein the first body location of the patient is proximal to a heart of the patient and the second body location is away from the heart of the patient and proximal to one of a carotid artery of the patient, a radial artery of the patient, or a femoral artery of the patient.

10. The method of claim 8, wherein the cardiac signal sensor comprises one or more of an electrocardiogram (ECG) sensor, an acoustic sensor or an inertial motion sensor.

11. The method of claim 8, further comprising:
filtering the pulse wave data based on the cardiac signal data.

12. The method of claim 11, wherein filtering the pulse wave data based on the cardiac signal data comprises synchronizing the pulse wave data and the cardiac signal data.

13. The method of claim 8, wherein generating the user-perceptible indication that the patient is not exhibiting PEA comprises rendering a visual indication that the patient is not exhibiting PEA to a display.

14. The method of claim 8, further comprising:
receiving additional pulse wave data generated by one or more of an inertial motion sensor or an acoustic sensor of a third patient-wearable device that is attached to a third body location of the patient; and
processing the additional pulse wave data to determine that the additional pulse wave data includes one or more pulse waveforms that respectively correspond to the identified one or more cardiac waveforms;
wherein generating the user-perceptible indication that the patient is not exhibiting PEA comprises generating the user-perceptible indication that the patient is not exhibiting PEA in response to at least determining that either one of the pulse wave data or the additional pulse wave data includes one or more pulse waveforms that respectively correspond to the identified one or more cardiac waveforms.

15. A method for determining and indicating that a patient is exhibiting pulseless electrical activity (PEA), comprising:
receiving cardiac signal data generated by one or more cardiac signal sensors of a first patient-wearable device that is attached to a first body location of the patient;
receiving pulse wave data generated by one or more of an inertial motion sensor or an acoustic sensor of a second patient-wearable device that is attached to a second body location of the patient;
processing the cardiac signal data to identify one or more cardiac waveforms thereof;
processing the pulse wave data to determine that the pulse wave data does not include one or more pulse waveforms that respectively correspond to the identified one or more cardiac waveforms; and
generating a user-perceptible indication that the patient is exhibiting PEA in response to at least determining that the pulse wave data does not include one or more pulse waveforms that respectively correspond to the identified one or more cardiac waveforms.

16. The method of claim 15, wherein the first body location of the patient is proximal to a heart of the patient and the second body location is away from the heart of the patient and proximal to one of a carotid artery of the patient, a radial artery of the patient, or a femoral artery of the patient.

17. The method of claim 15, wherein the cardiac signal sensor comprises one or more of an electrocardiogram (ECG) sensor, an acoustic sensor or an inertial motion sensor.

18. The method of claim 15, further comprising:
filtering the pulse wave data based on the cardiac signal data.

19. The method of claim 15, wherein generating the user-perceptible indication that the patient is exhibiting PEA comprises rendering a visual indication that the patient is exhibiting PEA to a display.

20. The method of claim 15, further comprising:
receiving additional pulse wave data generated by one or more of an inertial motion sensor or an acoustic sensor of a third patient-wearable device that is attached to a third body location of the patient; and
processing the additional pulse wave data to determine that the additional pulse wave data does not include one or more pulse waveforms that respectively correspond to the identified one or more cardiac waveforms;
wherein generating the user-perceptible indication that the patient is exhibiting PEA comprises generating the user-perceptible indication that the patient is exhibiting PEA in response to at least determining that both the pulse wave data and the additional pulse wave data do not include one or more pulse waveforms that respectively correspond to the identified one or more cardiac waveforms.
